(19) United States
(12) Patent Application Publication
(10) Pub. No.: US 2023/0304048 A1
(43) Pub. Date: Sep. 28, 2023

(54) RECOMBINANT MICROORGANISMS AND PROCESS

(71) Applicant: MacQuarie University, North Ryde, New South Wales (AU)

(72) Inventors: Robert Willows, North Ryde (AU); Louise Brown, North Ryde (AU); Natalie Curach, North Ryde (AU); Ante Jerkovic, North Ryde (AU); Kerstin Petroll, North Ryde (AU); Jocelyn Johns, North Ryde (AU); Samuel King, North Ryde (AU); Ari Edmonds, North Ryde (AU)

(21) Appl. No.: 17/916,137

(22) PCT Filed: Mar. 31, 2021
(86) PCT No.: PCT/AU2021/050290

§ 371 Date: Sep. 30, 2022

(30) Foreign Application Priority Data
Mar. 31, 2020 (AU) 2020900990

(51) Int. Cl. C12P 3/00; C12N 15/52; C12N 15/70; C12N 9/02; C12N 9/04

(52) U.S. Cl. CPC C12P 3/00; C12N 15/52; C12N 15/70; C12N 9/0067; C12Y 112/07002; C12N 9/0095; C12Y 118/01002; C12N 9/0006; C12Y 101/01; C12N 2800/101; H01M 8/16

(57) ABSTRACT

The present invention relates to recombinant microorganisms for producing biological hydrogen. In addition, the invention relates to nucleic acid constructs and processes for modifying microorganisms for enabling the production of hydrogen therefrom.

### CROSS REFERENCE TO RELATED APPLICATIONS

This application is a U.S. National Stage Application filed under 35 U.S.C. § 371, based on International Patent Application No. PCT/AU2021/050290, filed on Mar. 31, 2021, which claims priority to AU Patent Application No. 2020900990, filed on Mar. 31, 2020. The entire contents of each of the above applications, including any drawings and sequence listings, are incorporated herein by reference.

### FIELD OF THE INVENTION

The present invention relates to processes for producing hydrogen, nucleic acid constructs and recombinant microorganisms for producing hydrogen.

### BACKGROUND OF THE INVENTION

There has been an increasing interest in alternative fuels due to rising petroleum costs, escalating diplomatic tensions with oil producing countries, and the rising levels of greenhouse gases in the atmosphere. Hydrogen has enormous potential to serve as a non-polluting fuel, thereby alleviating the environmental and political concerns associated with fossil energy utilization. Thus, efforts to identify a candidate for replacing or supplementing fossil fuels as a source of clean energy have focused on the production of molecular hydrogen.

Key to a hydrogen economy is finding an efficient, inexpensive, and renewable process for the production of hydrogen while also achieving the equally important goal of economically converting hydrogen into usable energy.

One approach to the production of hydrogen on a commercial scale is the exploitation of photobiological production of hydrogen by eukaryotic organisms. For example, green algae respond to anaerobic stress by switching the oxidative pathway to a fermentative metabolism.

The ability of green algae, such as Chlamydomonas reinhardtii, to produce hydrogen from water has long been recognized. This reaction is catalyzed by a reversible hydrogenase, an enzyme that is induced in the cells after exposure to a short period of anaerobiosis. Thus, the use of algal bioreactors has been one approach to producing hydrogen. However, the activity of the hydrogenase is rapidly lost when cells are illuminated because of the immediate inactivation of the reversible hydrogenase by photosynthetically generated O₂.

Other approaches for the production of hydrogen include the generation of recombinant microorganisms, and the fermentation of a carbohydrate feedstock by those microorganisms. In some examples, hydrogenases from bacteria, archaea and algae have been expressed in E. coli, although expression of the exogenous enzymes in E. coli has been complicated by low expression rates and protein instability, despite codon optimisation.

Various hydrogenases have been proposed as candidates for use in such fermentation approaches. For example, the [NiFe] hydrogenase from the purple bacterium Allochromatiiim vinosum is a remarkably active electrocatalyst. Though [NiFe]-hydrogenases exhibit promise, there remain problems associated with use of these and other hydrogenase enzymes. The stability of hydrogenases has been one of the major disadvantages in their use in enzyme fuel cells. Furthermore, though the enzymes demonstrate less susceptibility to CO poisoning than does platinum, commercial use requires further improvement in terms of both the sensitivity to CO as well as to oxygen. In addition, the lack of hydrogenase availability in large quantities limits their potential application in enzyme fuel cells. Therefore, production of stable hydrogenase in large quantities and with desired catalytic properties will greatly enhance the application of this interesting bioelectrocatalyst for hydrogen fuel.

Algal bioreactors are expensive to scale up due to a number of light capture and hydrogen capture technical barriers. The rate of hydrogen production is also an important consideration as hydrogen is difficult to contain and collect at low volumes and concentrations. Algal systems produce hydrogen at very low rates and require nutrient limitation to start production. To date, both the production rates and the yields of hydrogen produced by either engineered microalgae or via fermentation of carbohydrate feedstock have been too slow and low, respectively, to be commercially viable.

There is therefore a need for improved processes for the generation of hydrogen.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The invention relates generally to expression vectors, microorganisms, methods and reactor systems to produce hydrogen and active hydrogenase enzymes for energy and electricity-generating applications. The expression vectors and microorganisms can be used in culture methods to produce the products of interest. Both the hydrogen and active hydrogenase products can be incorporated into a system such as, for example, a fuel cell system for producing electricity from hydrogen.

In a first aspect, the present invention provides a recombinant microorganism for producing hydrogen gas, wherein the microorganism comprises:
exogenous nucleic acid sequences encoding one or more proteins for enabling the microorganism to produce hydrogen,
   wherein the one or more proteins comprise an Fe—Fe-dependent hydrogenase and optionally at least one assembly protein for enabling maturation and activation of the hydrogenase;
   wherein the nucleic acid sequences are operably linked to one or more promoters for enabling expression of the nucleic acid sequences in the microorganism, and
   wherein the exogenous nucleic acid sequences are codon optimised to provide for optimised expression of the hydrogenases in the microorganism.

Preferably, the Fe—Fe hydrogenase is a member of the A1 class of Fe—Fe hydrogenases.

Preferably, the Fe—Fe-dependent hydrogenase is HydA (Hyd1) or a functionally equivalent homolog or derivative thereof.

In preferred embodiments, the Fe—Fe-dependent hydrogenase comprises the amino acid sequence of the HydA protein selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, Peptoclostridium bifermentans Clostridium arbusti, Pseudoflavonifractor capillosus, Lachnoclostridium citroniae, Lachnoclostridium clostridioforme, Pelosinus fermentans, Thermodesulfovibrio islandicus, Sutterella wadsworthensis, Clostridium beijerinckii, Fusobacterium ulcerans, Clostridium tyrobutyricum, Clostridium perfringens, Cetobacterium somerae, Clostridium beijerinckii, Clostridium colicanis, Clostridium intestinale, Clostridium chauvoei, Cellulomonas fimi, Ruminiclostridium thermocellum, Naegleria gruberi, Chlorella variabilis, Fervidobacterium nodosum, Thermotoga petrophila, Thermotoga lettingae, Thiomicrospira pelophila, Caldatribacterium califomiense, Fusobacterium necrophorum, Omnitrophus fodinae, Syntrophothermus lipocalidus, Ammonifex degensii, Desulfotomaculum hydrothermale, Fusobacterium mortiferum, Desulfotomaculum kuznetsovii, and Lachnoclostridium phytofermentans or functionally equivalent homologs or derivatives thereof. Preferably, the HydA protein is selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, and Peptoclostridium bifermentans, and functionally equivalent homologs thereof. More preferably, the HydA protein is from the Chlamydomonas reinhardtii or a functionally equivalent homolog or derivatives thereof.

In certain embodiments, the microorganism may be provided, during culturing of the microorganism, with one or more factors for enabling maturation and activation of the hydrogenase. Preferably the one or more factors is in the form of a small molecule. Examples of factors for enabling maturation and activation of the hydrogenase are [2Fe]-subsite mimetics containing an azadithiolate bridge. Such factors are described, for example in Esselborn et al., (2013) Nat Chem Biol 9 (10):607-609, and Berggren et al., (2013) Nature, 499: 66-69 the contents of which are incorporated herein by reference.

Preferably, the exogenous nucleic acid sequences encode at least one assembly protein for enabling maturation and activation of the hydrogenase, wherein the at least one protein is selected from the group consisting of: HydEF and/or HydG. More preferably, the exogenous nucleic acid sequences comprise sequences encoding both assembly proteins HydEF and HydG. In a particularly preferred embodiment, the HydEF and HydG proteins comprise the amino acid sequence of the HydEF and HydG proteins from Chlamydomonas reinhardtii or functionally equivalent homologs or derivatives thereof.

Accordingly, in a preferred embodiment, the present invention provides a recombinant microorganism for producing hydrogen gas, wherein the microorganism comprises:
exogenous nucleic acid sequences encoding one or more proteins for enabling the microorganism to produce hydrogen,
   wherein the one or more proteins comprise an Fe—Fe-dependent hydrogenase HydA, or a functionally equivalent homolog or derivative thereof, and the assembly proteins HydEF and HydG from Chlamydomonas reinhardtii, or functionally equivalent homologs or derivatives thereof;
   wherein the nucleic acid sequences are operably linked to one or more promoters for enabling expression of the nucleic acid sequences in the microorganism, and
   wherein the exogenous nucleic acid sequences are codon optimised to provide for optimised expression of the hydrogenases in the microorganism.

In any embodiment, the microorganism further comprises nucleic acid sequences encoding the proteins Ferredoxin NADP reductase (FNR) and ferredoxin (encoded by petF), or functionally equivalent homologs or derivatives thereof.

Preferably the source of the FNR is a Flavin containing ferredoxin reductase that utilises NADPH as the reducing agent to reduce Ferredoxin. More preferably, the ferredoxin protein is from Chlamydomonas reinhardtii and the FNR is any FNR capable of reducing the Ferrodoxin from Chlamydomonas reinhardtii. In a particularly preferred embodiment, the FNR and Ferrodoxin proteins comprise the amino acid sequences from Chlamydomonas reinhardtii or functionally equivalent homologs or derivatives thereof.

The recombinant microorganism may be any microorganism suitable for use of expression of recombinant proteins. In certain embodiments, the recombinant microorganism is selected from the group consisting of: Escherichia coli, Bacillus subtilis, Lactobacillus sp., or a Streptococcus sp., In preferred embodiments, the microorganism is a strain of Escherichia coli (E. coli).

In certain embodiments, the recombinant microorganism is partially or completely inactivated and/or non-viable.

In any embodiment, the exogenous nucleic acid sequences are provided in one or more polynucleotide constructs. In a preferred embodiment, the exogenous nucleic acid sequences encoding HydEF, HydG, HydA, and optionally Ferredoxin and FNR are provided in a single polynucleotide construct. In alternative embodiments, the nucleic acid sequences encoding the proteins are provided in separate polynucleotide constructs.

In a preferred embodiment, the present invention provides an E. coli cell comprising a recombinant construct encoding a cluster of protein that enable the cell to produce hydrogen, wherein the cluster of proteins comprises, consists or consists essentially of the polypeptides HydEF, HydG, HydA, ferredoxin and FNR from Chlamydomonas reinhardtii. In a preferred embodiment, the recombinant construct comprises, consists or consists essentially of the sequence set forth in SEQ ID NO: 10.

In further embodiments, the microorganism comprises one or more genetic modifications for redirecting carbon utilisation into the pentose phosphate pathway. The modification may result in the reduction or inhibition of activity of a protein that directs carbon towards the glycolytic pathway, thereby redirecting carbon utilisation towards the pentose phosphate pathway.

For example, the microorganism may be further modified to reduce or inhibit the activity or levels of one or more endogenous proteins selected from the group consisting of: phosphofructokinase, pyruvate kinase, glycerate mutase, glyceraldehyde-3-phosphate dehydrogenase, 6-phosphogluconoate dehydratase, and 2-keto-3-deoxy-6-phosphogluconate aldolase. These proteins are encoded by the genes pfkA, pps, gpmA/gpmM, gapA, edd and eda, respectively.

Preferably, the microorganism is genetically modified to delete or reduce expression of one or more of the genes pfkA, pps, gpmA/gpmM, gapA, edd and eda, encoding phosphofructokinase, pyruvate kinase, glycerate mutase, glyceraldehyde-3-phosphate dehydrogenase, 6-phosphogluconoate dehydratase and 2-keto-3-deoxy-6-phosphogluconate aldolase respectively. The modification may be any modification that partially or completely reduces expression of the gene. Where there is a partial reduction in expression, the expression may be reduced by at least 5%, at least 10%, at least 20%, at least 30%, at least 40%, at least 50%, at least 60%, at least 70%, at least 80%, at least 90% or more, compared to the expression in the wild-type microorganism of the same strain.

The genetic modification may be made using a CRISPR-Cas9 system or other genome modification system (such as lambda red recombinase) to partially or completely inhibit expression of the one or more genes. The genetic modification may result in the introduction of a complete or partial loss-of-function mutation in the gene, preferably a complete loss-of-function mutation. The modification may be the complete or partial excision of the gene sequence.

In certain embodiments, only one of pfkA, pps, gpmA/gpmM, gapA, edd and eda genes are deleted or knocked-down. Preferably pfkA or gpmA is deleted or knocked-down. In further embodiments, the microorganism is genetically modified to delete or reduce expression of two, three, four, five or all of the genes pfkA, pps, gpmA/gpmM, gapA, edd and eda. In certain embodiments, the genetic modification results in deletion or reduction in expression of: pfkA and gpmA; or edd and eda; or gpmM, edd and eda; or gpmA, edd and eda; or gpmM, edd, eda and pfkA; or gpmA, edd, eda and pfkA or all of pfkA, pps, gpmA/gpmM, edd and eda.

In still further embodiments, the microorganism is genetically modified to increase the level or activity of one or more proteins of the pentose phosphate pathway.

Preferably the one or more proteins is selected from the group consisting of: phosphoglucomutase, glucose-6-phosphate dehydrogenase, 6-phosphogluconolactonase, 6-phosphogluconate dehydrogenase, transketolase and transaldolase. These proteins are encoded by the genes pgm, zwf, pgl, gnd, tktB or tktA, and talA or talB, respectively. In a particularly preferred embodiment, the protein is glucose-6-phosphate dehydrogenase.

In further embodiments, the level or activity of endogenous NAD kinase (NADK, encoded by yfjB) and/or soluble pyridine nucleotide transhydrogenase (UdhA, encoded by sthA) is increased.

Preferably the increased level, or activity, of the proteins of the microorganism is accomplished by increasing expression of nucleic acid sequences encoding the one or more proteins of the pentose phosphate pathway, such that the level of the protein produced by the microorganism is increased relative to a wild-type microorganism of the same strain. In alternative embodiments, the increased level or activity of the proteins is accomplished by the introduction or one or more point mutations which result in increased activity of the protein.

In a preferred embodiment, the gene encoding glucose-6-phosphate dehydrogenase, zwf is overexpressed. In further embodiments, the gene encoding 6-phosphogluconate dehydrogenase, gnd is overexpressed. In other embodiments, the gene encoding 6-phosphogluconolactonase, pgl, is overexpressed.

Overexpression and increased levels or activity of phosphoglucomutase, glucose-6-phosphate dehydrogenase, 6-phosphogluconolactonase, 6-glucophosphonate dehydrogenase, transketolase and transaldolase can optionally be accomplished by modification of the promoter sequences of one or more of pgm, zwf, pgl, gnd, tktB or tktA, and talA or talB. In certain embodiments, the endogenous promoters for one or more of pgm, zwf, pgl, gnd, tktB or tktA, and, talA or talB is replaced with an exogenous promoter for increasing expression of the gene. In certain embodiments, the endogenous promoter for one or more of pgm, zwf, pgl, gnd, tktB or tktA, and talA or talB is replaced with an endogenous promoter which regulates expression of a different gene in the microorganism. In alternative embodiments, the endogenous promoter for one or more of pgm, zwf, pgl, gnd, tktB or tktA, and talA or talB is replaced with an exogenous promoter. The exogenous promoter may regulate expression of a homologous gene in a non-cognate microorganism or may regulate expression of a non-homologous protein in a non-cognate microorganism.

In particularly preferred embodiments, the endogenous promoter for one or more of pgm, zwf, pgl, gnd, tktB or tktA, and talA or talB is replaced with a promoter selected from the group consisting of: the osmY promoter, the gapA promoter, the nirB promoter and the nar promoter.

Still further, the host cell may be transformed with a recombinant construct which encodes a heterologous protein from another microbial species, for example for the purpose of increasing the level or activity of the relevant enzyme of the pentose phosphate pathway.

The recombinant construct may enable expression of the exogenous gene concomitantly with expression of the endogenous gene. Alternatively, the recombinant construct may be stably introduced into the microorganism genome, such that the endogenous gene sequence is replaced with the exogenous gene sequence.

Overexpression and increased levels or activity of phosphoglucomutase, glucose-6-phosphate dehydrogenase, 6-phosphogluconolactonase, 6-glucophosphonate dehydrogenase, transketolase and transaldolase can be accomplished by supplementation or replacement of the endogenous gene encoding said protein, with an exogenous gene encoding a homologous protein. In certain examples, the endogenous zwf gene is replaced with the zwf gene from Zymomonas mobilis. Preferably, the microorganism is E. coli and the E. coli zwf gene is replaced with the zwf gene from Zymomonas mobilis. In still further examples, the gnd gene is replaced with the gnd gene from Corynebacterium glutamicum. Preferably the microorganism is E. coli the gene encoding transketolase and transaldolase is supplemented or replaced with the homologous gene from Saccharomyces cerevisiae. Preferably, the microorganism is E. coli and the E. coli gnd gene is replaced with the gnd gene from Corynebacterium glutamicum. Further still, the endogenous gapA gene (encoding glyceraldehyde-3-phosphate dehydrogenase) is replaced with the gapC gene from Clostridium aceteobutylicum. Preferably, the microorganism is E. coli and the E. coli gapA gene is replaced with the gapC gene from Clostridium aceteobutylicum.

Preferably expression of the one or more genes encoding phosphoglucomutase, glucose-6-phosphate dehydrogenase, 6-phosphogluconolactonase, and 6-glucophosphonate dehydrogenase is increased by at least about 1.5-fold, at least about 2-fold, at least about 3-fold, at least about 5-fold or more.

In still further embodiments, the recombinant microorganism is modified so as to metabolise sucrose for energy consumption. In embodiments where the microorganism is E. coli, the microorganism is preferably genetically modified to express cscA and cscB genes, encoding sucrose hydrolase, and sucrose permease respectively from strains of E. coli that metabolise sucrose. Further still, the E. coli microorganism may be genetically modified to increase the levels or activity of endogenous E. coli phosphoglucomutase (pgm) or xylose isomerase (xyIA). The genetic modification may be to increase expression of the endogenous gene (for example, by modification of the promoter region) or by introduction and expression of an exogenous nucleic acid encoding the gene.

In further embodiments, the microorganism is modified to express sucrose phosphorylase from Leuconostoc mesenteroides

In any embodiment described herein requiring expression of an exogenous gene, the gene may be codon optimised for expression in the microorganism.

Where the microorganism is a recombinant E. coli microorganism, the microorganism may be any strain of E. coli capable of expressing an exogenous nucleic acid sequence. In certain preferred embodiments, the E. coli strain is selected from any K12 derived or W derived strain. In certain embodiments, the E. coli strain is selected from the group consisting of: DH5a (DH5alpha).

In a further aspect, the present invention provides a method for producing hydrogen gas, the method comprising:
providing a host cell comprising one or more recombinant polynucleotides comprising nucleic acid sequences encoding an Fe—Fe-dependent hydrogenase, wherein the nucleic acid sequences are operably linked to one or more promoters for enabling expression of the nucleic acid sequences in the microorganism,
contacting the host cell with an exogenous factor for enabling maturation and activation of the hydrogenase;
culturing the host cell under suitable conditions for enabling production of hydrogen therefrom.

Preferably the one or more factors is in the form of a small molecule. Examples of factors for enabling maturation and activation of the hydrogenase are [2Fe]-subsite mimetics containing an azadithiolate bridge. Such factors are described, for example in Esselborn et al., (2013) Nat Chem Biol 9 (10):607-609, and Berggren et al., (2013) Nature, 499: 66-69 the contents of which are incorporated herein by reference.

Further, the present invention provides a method for producing hydrogen gas, the method comprising:
providing a host cell comprising one or more recombinant polynucleotides comprising nucleic acid sequences encoding an Fe—Fe-dependent hydrogenase and at least one assembly protein for enabling maturation and activation of the hydrogenase;
   wherein the nucleic acid sequences are operably linked to one or more promoters for enabling expression of the nucleic acid sequences in the microorganism, and
   wherein the exogenous nucleic acid sequences are codon optimised to provide for optimised expression of the hydrogenases in the microorganism.

In another aspect, the present invention provides a method for producing hydrogen gas, the method comprising:
providing one or more polynucleotides comprising nucleic acid sequences encoding an Fe—Fe-dependent hydrogenase and optionally at least one assembly protein for enabling maturation and activation of the hydrogenase, wherein the nucleic acid sequences are operably linked to a promoter for enabling expression of the nucleic acid sequences and wherein the nucleic acid sequences are codon optimised for expression in a heterologous host cell;
providing a heterologous host cell;
transforming or transfecting the host cell with the polynucleotide(s);
providing cell culture media; and
culturing the transformed or transfected host cell in the cell culture media under conditions sufficient for expression of the polynucleotide.

In still a further aspect, the present invention provides a method for maximising the expression of a hydrogen-generating Fe—Fe hydrogenase, preferably A1 Fe—Fe hydrogenase in a heterologous host cell, the method comprising:
providing a polynucleotide comprising nucleic acid sequences encoding an Fe—Fe-dependent hydrogenase and at least one assembly protein for enabling maturation and activation of the hydrogenase, wherein the nucleic acid sequences are operably linked to a promoter and are codon optimised for enabling expression of the nucleic acid sequences in a heterologous host cell;
providing a heterologous host cell;
transforming or transfecting the host cell with the polynucleotide;
providing cell culture media; and
culturing the transformed or transfected host cell in the cell culture media under conditions sufficient for expression of the polynucleotide.

Preferably the Fe—Fe-dependent hydrogenase is a class A1 Fe—Fe hydrogenase.

Preferably, the Fe—Fe-dependent hydrogenase is HydA (Hyd1) or a functionally equivalent homolog or derivative thereof.

In preferred embodiments, the Fe—Fe-dependent hydrogenase comprises the amino acid sequence of the HydA protein selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, Peptoclostridium bifermentans Clostridium arbusti, Pseudoflavonifractor capillosus, Lachnoclostridium citroniae, Lachnoclostridium clostridioforme, Pelosinus fermentans, Thermodesulfovibrio islandicus, Sutterella wadsworthensis, Clostridium beijerinckii, Fusobacterium ulcerans, Clostridium tyrobutyricum, Clostridium perfringens, Cetobacterium somerae, Clostridium beijerinckii, Clostridium colicanis, Clostridium intestinale, Clostridium chauvoei, Cellulomonas fimi, Ruminiclostridium thermocellum, Naegleria gruberi, Chlorella variabilis, Fervidobacterium nodosum, Thermotoga petrophila, Thermotoga lettingae, Thiomicrospira pelophila, Caldatribacterium califomiense, Fusobacterium necrophorum, Omnitrophus fodinae, Syntrophothermus lipocalidus, Ammonifex degensii, Desulfotomaculum hydrothermale, Fusobacterium mortiferum, Desulfotomaculum kuznetsovii, and Lachnoclostridium phytofermentans or functionally equivalent homologs or derivatives thereof. Preferably, the HydA protein is selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, and Peptoclostridium bifermentans, and functionally equivalent homologs thereof. More preferably, the HydA protein is from the Chlamydomonas reinhardtii or a functionally equivalent homolog or derivatives thereof.

Preferably, the at least one assembly protein comprises a protein from the group consisting of: HydEF and/or HydG. More preferably, the exogenous nucleic acid sequences comprise sequences encoding both assembly proteins HydEF and HydG. In a particularly preferred embodiment, the HydEF and HydG proteins comprise the amino acid sequence of the HydEF and HydG proteins from Chlamydomonas reinhardtii or functionally equivalent homologs or derivatives thereof.

Accordingly, in preferred embodiments, the present invention provides a method for producing hydrogen gas, the method comprising:
providing a host cell comprising one or more recombinant polynucleotides comprising nucleic acid sequences encoding the Chlamydomonas reinhardtii polypeptides HydEF, HydG and HydA,
   wherein the nucleic acid sequences are operably linked to a promoter for enabling expression of the nucleic acid sequences,
   wherein the nucleic acid sequences are codon optimised for expression in a heterologous host; and
culturing said host cell in a suitable culture medium under conditions to effect expression of the polynucleotides.

In another aspect, the present invention provides a method for producing hydrogen gas, the method comprising:
providing one or more polynucleotides comprising nucleic acid sequences encoding the Chlamydomonas reinhardtii polypeptides HydEF, HydG and HydA, wherein the nucleic acid sequences are operably linked to a promoter for enabling expression of the nucleic acid sequences and wherein the nucleic acid sequences are codon optimised for expression in a heterologous host cell;
providing a host cell;
transforming or transfecting the host cell with the polynucleotide(s);
providing cell culture media; and
culturing the transformed or transfected host cell in the cell culture media under conditions sufficient for expression of the polynucleotide.

In still a further aspect, the present invention provides a method for maximising the expression of a hydrogen-generating Fe—Fe hydrogenase from Chlamydomonas reinhardtii in a heterologous host cell, the method comprising:
providing a polynucleotide comprising nucleic acid sequences encoding the Chlamydomonas reinhardtii polypeptides HydEF, HydG and HydA, wherein the nucleic acid sequences are operably linked to a promoter and are codon optimised for enabling expression of the nucleic acid sequences in a heterologous host cell;
providing a heterologous host cell;
transforming or transfecting the host cell with the polynucleotide;
providing cell culture media; and
culturing the transformed or transfected host cell in the cell culture media under conditions sufficient for expression of the polynucleotide.

Preferably the heterologous host cell is an E. coli cell and the nucleic acid sequences are codon optimised for expression in E. coli. Preferably the promoters in the polynucleotide are for expression of the polynucleotides in E. coli.

In a preferred embodiment of any of the above aspects, the recombinant polynucleotide(s) comprise nucleic acid sequences encoding Ferredoxin NADP reductase and Ferredoxin, or functionally equivalent homologs or derivatives thereof.

Preferably the source of the FNR is a Flavin containing ferredoxin reductase that utilises NADPH as the reducing agent to reduce Ferredoxin. More preferably, the ferredoxin protein is from Chlamydomonas reinhardtii and the FNR is any FNR capable of reducing the Ferrodoxin from Chlamydomonas reinhardtii. In a particularly preferred embodiment, the FNR and Ferrodoxin proteins comprise the amino acid sequences from Chlamydomonas reinhardtii or functionally equivalent homologs or derivatives thereof.

The host cell may be any microorganism suitable for use of expression of recombinant proteins. In certain embodiments, the host cell is selected from the group consisting of: Escherichia coli, Bacillus subtilis, Lactobacillus sp., or a Streptococcus sp., In preferred embodiments, the microorganism is a strain of Escherichia coli (E. coli).

In certain embodiments, the host cell is partially or completely inactivated and/or non-viable.

As used herein, the combination of nucleic acid sequences encoding HydEF, HydG, HydA, Ferredoxin NADP reductase and Ferredoxin may also be referred to as the hydrogen producing gene cluster (HPGC).

In any embodiment, the above methods may further comprise utilising a genetically modified host cell, or modifying the microorganism or host cell, or contacting the microorganism or host cell with an agent to reduce or inhibit the activity or levels of one or more endogenous host cell proteins selected from the group consisting of: phosphofructokinase, pyruvate kinase, glycerate mutase, 6-phosphogluconoate dehydratase and 2-keto-3-deoxy-6-phosphogluconate aldolase.

The agent for reducing or inhibiting the activity or levels of one or more of phosphofructokinase, pyruvate kinase, glycerate mutase, 6-phosphogluconoate dehydratase and 2-keto-3-deoxy-6-phosphogluconate aldolase may be selected from: a small molecule, a peptide, an antibody, an interfering RNA, for example an antisense RNA, microRNA, shRNA, siRNA, that can reduce the activity or levels of one or more of the proteins.

In preferred embodiments, the methods comprise contacting or having contexted the the microorganism or host cell with an agent which genetically modifies the microorganism or host cell such that the levels or activity of one or more of the pfkA, pps, gpmA/gpmM, gapA, edd and eda (encoding phosphofructokinase, pyruvate kinase, glycerate mutase, glyceraldehyde-3-phosphoate dehydrogenase, 6-phosphogluconoate dehydratase and 2-keto-3-deoxy-6-phosphogluconate aldolase, respectively) are partially or completely reduced. For example, the agent may comprise a gRNA molecule for use in combination with a CRISPR-Cas9 or other genome-editing system (such as lambda red recombinase) for deleting part or all of the gene.

In still further embodiments, the methods further comprise genetically modifying or having modified the microorganism or host cell to increase the level or activity of one or more proteins of the pentose phosphate pathway. Preferably the one or more proteins is selected from the group consisting of: phosphoglucomutase, glucose-6-phosphate dehydrogenase, 6-phosphogluconolactonase, 6-phosphogluconate dehydrogenase, transketolase and transaldolase. These genes are encoded by the genes pgm, zwf, pgl, gnd, tktB or tktA, and, talA or talB, respectively. In a particularly preferred embodiment, the protein is glucose-6-phosphate dehydrogenase.

In further embodiments, the level or activity of endogenous NAD kinase (NADK, encoded by yfjB) and/or soluble pyridine nucleotide transhydrogenase (UdhA, encoded by sthA) is increased.

Preferably the methods comprise modifying the microorganism or host cell to comprise nucleic acid sequences for overexpressing the genes encoding the one or more proteins of the pentose phosphate pathway, such that the level of the protein produced by the microorganism is increased relative to a wild-type microorganism of the same strain. In alternative embodiments, the increased level or activity of the proteins is accomplished by the introduction or one or more point mutations which result in increased activity of the protein.

In a preferred embodiment, the gene encoding glucose-6-phosphate dehydrogenase, zwf is overexpressed. In further embodiments, the gene encoding 6-phosphogluconate dehydrogenase, gnd is overexpressed. In other embodiments, the gene encoding 6-phosphogluconolactonase, pgl, is overexpressed. In other embodiments, the gene encoding glyceraldehyde-3-phosphate dehydrogenase, gapA, is overexpressed. Overexpression can optionally be accomplished by modification of the promoter sequences of one or more of pgm, zwf, pgl, gnd, tktB or tktA, and, talA or talB.

In certain embodiments, the endogenous promoters for one or more of pgm, zwf, pgl, gnd, tktB or tktA, and, talA or talB is replaced with an exogenous promoter for increasing expression of the gene. In certain embodiments, the endogenous promoter for one or more of pgm, zwf, pgl, gnd, tktB or tktA, and talA or talB is replaced with an endogenous promoter which regulates expression of a different gene in the microorganism. In alternative embodiments, the endogenous promoter for one or more of pgm, zwf, pgl, gnd, tktB or tktA, and talA or talB is replaced with an exogenous promoter. The exogenous promoter may regulate expression of a homologous gene in a non-cognate microorganism or may regulate expression of a non-homologous protein in a non-cognate microorganism.

In particularly preferred embodiments, the endogenous promoter for one or more of pgm, zwf, pgl, gnd, tktB or tktA, and talA or talB is replaced with a promoter selected from the group consisting of: the osmY promoter, the gapA promoter, the nirB promoter and the nar promoter.

Overexpression and increased levels or activity of phosphoglucomutase, glucose-6-phosphate dehydrogenase, 6-phosphogluconolactonase, 6-glucophosphonate dehydrogenase, transketolase and transaldolase may also be accomplished by replacement of the endogenous gene encoding said protein, with an exogenous gene encoding a homologous protein.

Preferably expression of the one or more genes encoding phosphoglucomutase, glucose-6-phosphate dehydrogenase, 6-phosphogluconolactonase, and 6-glucophosphonate dehydrogenase is increased by at least about 1.5-fold, at least about 2-fold, at least about 3-fold, at least about 5-fold or more.

In still further embodiments, the recombinant microorganism or host cell is modified so as to metabolise sucrose for energy consumption. In embodiments where the microorganism is E. coli, the microorganism is preferably genetically modified to express cscA and cscB genes, encoding sucrose hydrolase, and sucrose permease respectively from strains of E. coli that metabolise sucrose. Further still, the E. coli microorganism may be genetically modified to increase the levels or activity of endogenous E. coli phosphoglucomutase (pgm) or xylose isomerase (xylA). The genetic modification may be to increase expression of the endogenous gene (for example, by modification of the promoter region) or by introduction and expression of an exogenous nucleic acid encoding the gene.

In further embodiments, the microorganism is modified to express sucrose phosphorylase from Leuconostoc mesenteroides

In embodiments where the host cell is E. coli, the E. coli microorganism may be any strain of E. coli capable of expressing an exogenous nucleic acid sequence. In certain preferred embodiments, the E. coli strain is selected from any K12 derived or W derived strain. In certain embodiments, the E. coli strain is selected from the group consisting of: DH5α (DH5alpha).

In further embodiments, the methods described herein further comprise culturing the microorganism or host cell in conditions which are optimised for enabling expression of the hydrogen producing gene cluster (HPGC) described herein, and thereby increasing the production of hydrogen by the microorganism. In one example, the methods comprise culturing the host cell under anaerobic conditions. The skilled person will be familiar with methods for culturing cells under anaerobic conditions, including by the addition of a neutral gas as a reductant.

Further still, the culture conditions may include addition of ferric (iron III) or ferrous (iron II) to the culture medium. In preferred embodiments, the ferrous iron (Fe II) is added to the culture medium at a concentration of at least about 20 μM or greater, preferably no more than about 50 μM.

The culturing conditions are preferably performed at no more than 37° C., more preferably at less than about 35° C., less than about 32° C., most preferably at less than about 30° C.

The present invention also provides various nucleic acid constructs or polynucleotides for use in a system for generating molecular hydrogen.

In one embodiment, the invention provides a nucleic acid construct or polynucleotide comprising nucleotide sequence encoding the polypeptides HydEF, HydG and HydA, wherein the nucleic acid sequences are operably linked to a promoter for enabling expression of the nucleic acid sequences and wherein the nucleic acid sequences are codon optimised for expression in a heterologous host. Preferably, the nucleic acid sequences are codon optimised for expression in E. coli. Preferably, the HydEF and HydG polypeptides are from Chlamydomonas reinhardtii.

In preferred embodiments, the nucleic acid encoding the HydA protein in the nucleic acid constructs of the invention, encodes the amino acid sequence of the HydA protein from an organism selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, Peptoclostridium bifermentans Clostridium arbusti, Pseudoflavonifractor capillosus, Lachnoclostridium citroniae, Lachnoclostridium clostridioforme, Pelosinus fermentans, Thermodesulfovibrio islandicus, Sutterella wadsworthensis, Clostridium beijerinckii, Fusobacterium ulcerans, Clostridium tyrobutyricum, Clostridium perfringens, Cetobacterium somerae, Clostridium beijerinckii, Clostridium colicanis, Clostridium intestinale, Clostridium chauvoei, Cellulomonas fimi, Ruminiclostridium thermocellum, Naegleria gruberi, Chlorella variabilis, Fervidobacterium nodosum, Thermotoga petrophila, Thermotoga lettingae, Thiomicrospira pelophila, Caldatribacterium califomiense, Fusobacterium necrophorum, Omnitrophus fodinae, Syntrophothermus lipocalidus, Ammonifex degensii, Desulfotomaculum hydrothermale, Fusobacterium mortiferum, Desulfotomaculum kuznetsovii, and Lachnoclostridium phytofermentans or functionally equivalent homologs or derivatives thereof. Preferably, the HydA protein is selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, and Peptoclostridium bifermentans, and functionally equivalent homologs thereof. More preferably, the HydA protein is from the Chlamydomonas reinhardtii or a functionally equivalent homolog or derivatives thereof

The present invention also provides a polynucleotide comprising nucleic acid sequences encoding the hydrogen producing gene cluster (HPGC), wherein the HPGC comprises genes encoding HydEF, HydG, HydA, ferredoxin NADP reductase and ferredoxin. Preferably the nucleic acid encodes an FNR that is a Flavin containing ferredoxin reductase that utilises NADPH as the reducing agent to reduce Ferredoxin. More preferably, the nucleic acid encodes a ferredoxin protein from Chlamydomonas reinhardtii and encodes an FNR that is any FNR capable of reducing the Ferrodoxin from Chlamydomonas reinhardtii. In a particularly preferred embodiment, the polynucleotide comprises nucleic acids encoding FNR and Ferrodoxin proteins from Chlamydomonas reinhardtii or functionally equivalent homologs or derivatives thereof.

In a particularly preferred embodiment, the sequence of the polynucleotide comprising the HPGC comprises, consists, or consists essentially of the nucleotide sequence set forth in SEQ ID NOs: 10 or 30 to 40.

The present invention also provides a microorganism as described herein, when used, or for use in a system for producing hydrogen. Accordingly, the present invention provides a system for producing hydrogen, wherein the system comprises:
a culture or population of recombinant microorganisms as herein described;
a feedstock for use by the recombinant microorganisms to induce expression of one or more proteins for enabling production of hydrogen by the microorganisms.

Optionally, the system also comprises means for storing or transferring the hydrogen produced by the recombinant microorganisms.

Preferably, the feedstock is a carbohydrate-based feedstock, such as glucose or sucrose or any other carbohydrate source.

The present invention also provides a bioreactor for producing hydrogen, comprising: a vessel which comprises a hydrogen producing system as described herein, said system comprising a suspension of hydrogen generating microorganisms of the invention, a feedstock for providing a source of carbon for use by the recombinant microorganisms and means for separating or extracting hydrogen gas from said suspension.

The present invention also provides a microorganism as described herein, when used, or for use in a system for producing electricity. Accordingly, the present invention provides a system or device for producing electricity from hydrogen, wherein the system or device comprises:
a culture or population of recombinant microorganisms as herein described;
a feedstock for use by the recombinant microorganisms to induce expression of one or more proteins for enabling production of hydrogen by the microorganisms;
a hydrogen fuel cell;
means for transferring the hydrogen produced by the recombinant microorganisms to the hydrogen fuel cell.

The present invention also provides a method for producing electricity, the method comprising operating a system or device comprising a recombinant microorganism as described herein, or utilising hydrogen produced according to a method described herein.

The present invention also provides for use of a recombinant microorganism as herein described, in a system or device for producing electricity from hydrogen.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Hydrogen production in wild-type and mutant strains expressing the hydrogen producing gene cluster (HPGC). H₂ gas production after addition of 20 mM glucose to 50 mL Escherichia coli culture. DH5α without the hydrogen producing gene cluster (HPGC) makes no hydrogen under these conditions. The four strains DH5a with plasmid pHPGC; DH5a Δpfk with pHPGC; DH5a ΔgpmA with pHPGC; and DH5a with plasmid pH1-HEFG (pHPGC without petF-FNR), rapidly start to accumulate hydrogen after the addition of glucose. Hydrogen concentration in gas phase measured by gas chromatography.

FIG. 2: Accumulation of total organic acid fermentation products (succinate plus pyruvate plus lactate) in wild-type and mutant strains expressing the hydrogen producing gene cluster. Organic acid accumulation after addition of glucose for hydrogen production using HPGC.

FIG. 3: Schematic of exemplary device comprising microorganisms of the invention.

FIG. 4: Hydrogen production rates of various recombinant microorganisms containing pHPGC, relative to wild-type E. coli DH5α with pHPGC (control). Rate of hydrogen production (L/h) by E. coli genetically modified to reduce the flow of carbon from glucose through the lower section of the glycolytic pathway by deleting gpmM, ΔgpmA. Rate of hydrogen production increased with deletion of gpmA and gpmM. Increased expression of gnd and zwf also improved the rate of hydrogen production.

FIG. 5: Ratio of hydrogen to carbon dioxide produced by various recombinant microorganisms containing pHPGC, relative to wild-type E. coli DH5a with pHPGC (control). Increasing expression of zwf, Gp::zwf or reducing pfk activity, Δpfk, increases the ratio of hydrogen to CO₂ and hence flux through the pentose phosphate pathway. As gnd encodes a protein that is downstream of the protein encoded by zwf, the increase in activity of gnd, Gp::gnd, in this mutant has no significant effect on the ratio H₂ to CO₂ ratio compared to the wt DH5.

FIG. 6: Utilisation of Sucrose in making hydrogen by modified DH5a cscAB. DH5a cscAB with HPGC strain is able to utilise sucrose to make hydrogen like the positive control W strain with HPGC. Wild type DH5a with HPGC is unable to utilise sucrose and no hydrogen is detectably made when sucrose is supplied to this strain.

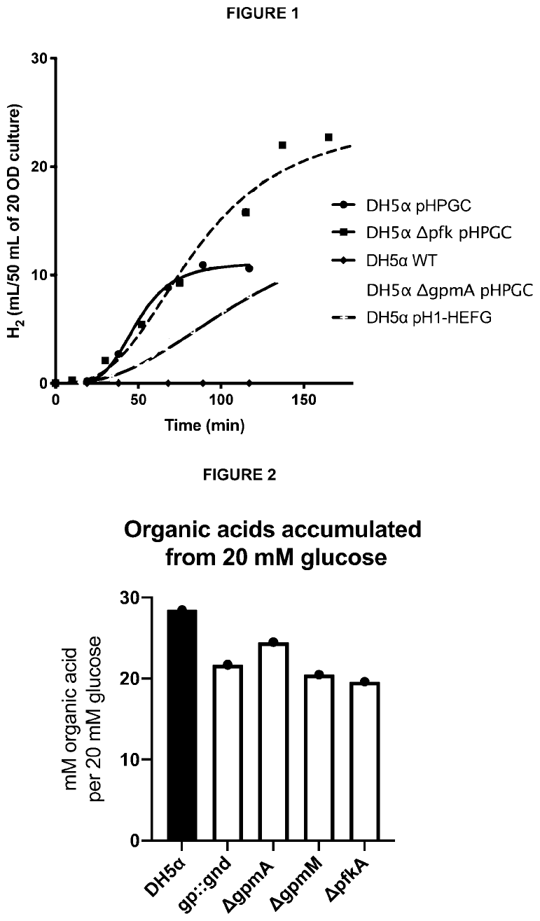

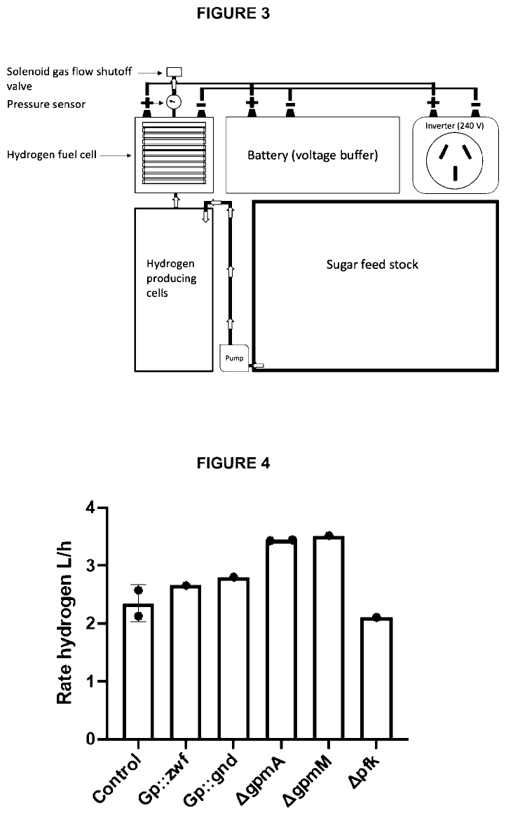

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

Microorganisms are able to synthesise molecular hydrogen using classes of enzymes known generally as hydrogenases. Seeking to harness the capacity of these enzymes to generate hydrogen, researchers have endeavoured to express hydrogenases from various microorganisms and algae in heterologous expression systems. Typically, this approach has involved screening various hydrogenases from algal and ‘extremophile microorganisms’. However, efforts to generate sufficient amounts of hydrogen using this type approach have been hampered by the instability of these hydrogenases and their cognate maturation proteins, and the resulting Fe—Fe hydrogenase enzyme complex, when expressed in heterologous organisms.

In order to address such limitations, others in the field have endeavoured to co-express maturation proteins from non-cognate organisms when expressing hydrogenases in heterologous organisms. However, this approach has generally failed to overcome the difficulties in obtaining sufficient levels of hydrogen for use in a commercial system.

Others have sought to focus on the type of hydrogenase being expressed, for example, seeking to express Ni—Fe hydrogenases rather than Fe—Fe hydrogenases. Such systems have been demonstrated to be functional in vitro and efficient at producing hydrogen utilising only the NADPH dependent Ni—Fe hydrogenase and a mixture of commercially available enzymes from the pentose phosphate pathway enzymes. However, such systems have not proved commercially viable due to the cost of providing the additional enzymes. Further, the rate of hydrogen production was too slow for commercial production.

The present inventors have identified a new approach for maximising production of molecular hydrogen from algal genes expressed in heterologous host cells. The approach adopted by the inventors allows for the stable production of an Fe—Fe hydrogenase complex from various microorganisms. Moreover, the inventors believe that their approach provides for increased rates of hydrogen production over time, and increased yield (production per input). The approach of the inventors represents a major advance over previous non-optimised approaches for generation of biological hydrogen.

Hydrogen Producing Gene Cluster

The invention includes providing a microorganism, as described herein, with various nucleic acid sequences encoding components of the molecular machinery required to produce hydrogen in that microorganism. In addition, the invention provides genetically modified microorganisms comprising those nucleic acid sequences.

In particular, the present invention includes the provision of a host cell with nucleic acid sequences (including recombinant polynucleotides) encoding the HydEF, HydG and HydA proteins. In preferred embodiments, the host cell is also provided with nucleic acids encoding Ferredoxin NADP reductase (FNR) and Ferredoxin (petF). It will be appreciated that hydrogen may be produced by a microorganism, as herein described, where the microorganism is modified to express a nucleic acid sequence encoding HydA, and wherein the microorganism comprises endogenous ferrodoxin.

Moreover, maturation of the hydrogenase may be accomplished using small molecules such as [2Fe]-subsite mimetics containing an azadithiolate bridge. Such factors are described, for example in Esselborn et al., (2013) Nat Chem Biol 9 (10):607-609, and Berggren et al., (2013) Nature, 499: 66-69 the contents of which are incorporated herein by reference.

However, in preferred embodiments, the microorganism is preferably modified to express the components of the HPGC as herein defined.

As used herein, the hydrogen producing gene cluster (HPGC) preferably comprises nucleic acid sequences encoding HydA, HydEF, HydG, ferredoxin NADP reductase and ferredoxin, wherein HydA refers to a Fe—Fe hydrogenase (preferably A1 subclass), and HydEF and HydG refer to the maturation and assembly complex of proteins required for formation of an active FeFe-hydrogenase.

As used herein HydA refers to any Fe—Fe-hydrogenase protein HydA, also referred to as iron hydrogenase, or iron hydrogenase HydA1 or Hyd1. This protein is encoded by the gene hyd1.

The skilled person will be familiar with methods for classification of different hydrogenases, including methods for determining whether a given hydrogenase is a Fe—Fe hydrogenase (including A1 class), as distinct from an Ni—Fe hydrogenase of an Fe-hydrogenase. Such methods are described for example in Søndergaard et al., (2016) Scientific Reports, 6:34212.

The HydA protein may be the HydA protein from a microorganism selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, Peptoclostridium bifermentans Clostridium arbusti, Pseudoflavonifractor capillosus, Lachnoclostridium citroniae, Lachnoclostridium clostridioforme, Pelosinus fermentans, Thermodesulfovibrio islandicus, Sutterella wadsworthensis, Clostridium beijerinckii, Fusobacterium ulcerans, Clostridium tyrobutyricum, Clostridium perfringens, Cetobacterium somerae, Clostridium beijerinckii, Clostridium colicanis, Clostridium intestinale, Clostridium chauvoei, Cellulomonas fimi, Ruminiclostridium thermocellum, Naegleria gruberi, Chlorella variabilis, Fervidobacterium nodosum, Thermotoga petrophila, Thermotoga lettingae, Thiomicrospira pelophila, Caldatribacterium califomiense, Fusobacterium necrophorum, Omnitrophus fodinae, Syntrophothermus lipocalidus, Ammonifex degensii, Desulfotomaculum hydrothermale, Fusobacterium mortiferum, Desulfotomaculum kuznetsovii, and Lachnoclostridium phytofermentans or functionally equivalent homologs or derivatives thereof.

Preferably, the HydA protein is selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, and Peptoclostridium bifermentans, and functionally equivalent homologs thereof. More preferably, the HydA protein is from Chlamydomonas reinhardtii or a functionally equivalent homolog or derivatives thereof.

Exemplary sequences of the Chlamydomonas reinhardtii protein sequence for HydA are provided under UniProt accession number Q9FYU1 and exemplary nucleic acid sequences encoding said protein may be found under accession number AJ308413, CAC83731.1 (EBI) and XP_001693376.1.

An exemplary nucleic acid sequence encoding HydA is provided in SEQ ID NO: 6. An exemplary promoter for enabling expression of hydA is provided in SEQ ID NO: 5.

Exemplary accession numbers providing sequence information for HydA from Volvox carteri, Giardia lamblia, Entamoeba nuttalli, Ilyobacter polytrophus, Trichomonas vaginalis, Megasphaera micronuciformis, Veillonella parvula, Veillonella atypica, and Peptoclostridium bifermentans include XP002956049, XP001709915, XP008860420, WP013388849 (and XP002948483), XP001330775, WP006942403, WP004697562, WP005375825 and WP021432477, respectively.

Exemplary codon optimised nucleic acid sequences (including restriction sites) encoding HydA from Volvox carteri, Giardia lamblia, Entamoeba nuttalli, Ilyobacter polytrophus, Trichomonas vaginalis, Megasphaera micronuciformis, Veillonella parvula, Veillonella atypica, and Peptoclostridium bifermentans are provided in SEQ ID NOs: 18 to 27.

As used herein, HydEF preferably refers to the Chlamydomonas reinhardtii Fe-hydrogenase assembly protein HydEF, also referred to as iron hydrogenase assembly protein HydEF. This protein is encoded by the gene hydEF. Exemplary sequences of the Chlamydomonas reinhardtii protein sequence for HydEF are provided under UniProt accession number Q6PSL5 and exemplary nucleic acid sequences encoding said protein may be found under accession numbers DS496119, EDP05198.1 (EBI) and XP_001691465.1.

An exemplary nucleic acid sequence encoding HydEF is provided in SEQ ID NO: 2. An exemplary promoter for enabling expression of hydEF is provided in SEQ ID NO: 1.

As used herein HydG refers to the Chlamydomonas reinhardtii Fe-hydrogenase assembly protein HydG, also referred to as iron hydrogenase assembly protein HydG. This protein is encoded by the gene hydG. Exemplary sequences of the Chlamydomonas reinhardtii protein sequence for HydG are provided under UniProt accession number Q6PSL4 and exemplary nucleic acid sequences encoding said protein may be found under accession number DS496119, EDP05052.1 (EBI) and XP_001691319.1.

An exemplary nucleic acid sequence encoding HydG is provided in SEQ ID NO: 4. An exemplary promoter for enabling expression of hydG is provided in SEQ ID NO: 3.

As used herein ferredoxin refers to the Chlamydomonas reinhardtii ferredoxin protein encoded by the petF gene. Exemplary sequences of the Chlamydomonas reinhardtii protein sequence for ferredoxin are provided under UniProt accession number A8IV40 and exemplary nucleic acid sequences encoding said protein may be found under accession number DS496124, EDP03827.1 (EBI) and XP_001692808.1.

An exemplary nucleic acid sequence encoding ferredoxin is provided in SEQ ID NO: 8. An exemplary promoter for enabling expression of petF is provided in SEQ ID NO: 7.

As used herein, Chlamydomonas reinhardtii ferredoxin NADP reductase (FNR) refers to EC:1.18.1.2. The protein is encoded by the gene petH or fnr1. Exemplary sequences of the Chlamydomonas reinhardtii protein sequence for FNR are provided under UniProt accession number A8J6Y8 and P53991 and exemplary nucleic acid sequences encoding said protein may be found under accession number DS496140, EDP00292.1 (EBI) and XP_001697352.1.

An exemplary nucleic acid sequence encoding FNR is provided in SEQ ID NO: 9.

In preferred embodiments, the nucleic acid sequences encoding the HydEF, HydG, HydA, Ferredoxin and FNR proteins are provided in a single polynucleotide construct. In one example, the polynucleotide has the nucleic acid sequence as set forth in SEQ ID NO: 10. Redirection oxidation of glucose towards pentose phosphate pathway

The inventors have found that isolation of the pentose phosphate pathway from the glycolytic pathway allows for optimal conversion of carbohydrate to hydrogen. Accordingly, in preferred embodiments, the microorganisms of the present invention are further modified to reduce or delete the expression of one or more of endogenous genes encoding phosphofructokinase, pyruvate kinase, glycerate mutase, 6-phosphogluconate dehydratase and 2-keto-3-deoxy-6-phosphogluconate aldolase. These proteins are encoded by the genes pfkA, pps, gpmA, gpmM, gapA, edd and eda, respectively,

Further, the methods of the invention include contacting the microorganism with one or more agents for inhibiting the activity or levels of one or more of the proteins phosphofructokinase, pyruvate kinase, glycerate mutase, 6-phosphogluconate dehydratase and 2-keto-3-deoxy-6-phosphogluconate aldolase.

As used herein, phosphofructokinase, (E.C. 2.7.1.11 and E.C. 2.7.1.105), also known as PFK is a kinase enzyme that phosphorylates fructose 6-phosphate in glycolysis. Phosphofructokinase catalyses the phosphorylation of fructose-6-phosphate to fructose-1,6-diphosphate, a key regulatory step in the glycolytic pathway.

As used herein pyruvate kinase, (E.C. 2.7.1.40) encoded by the pps gene, is an enzyme that catalyses the transfer of a phosphate group from phosphoenolpyruvate (PEP) to adenosine diphosphate (ADP), yielding one molecule of pyruvate and one molecule of ATP.

As used herein glycerate mutase, may refer to either the 2,3-bisphosphoglyerate-dependent (dPGM, GpmA) or the cofactor-independent (iPGM, GpmM) phosphoglycerate mutase. 2,3-bisphosphoglycerate-dependent phosphoglycerate mutase is encoded by the gpmA gene and catalyses the reaction 2-phospho-D-glycerate ↔3-phospho-D-glycerate. 2,3-bisphosphoglycerate-independent phosphoglycerate mutase (also known as gpmC; pgml; gpml or yibO) is encoded by the gpmM gene and catalyses the same reaction. The iPGM enzyme has significantly lower specific activity. Therefore, preferably pgmA is targeted for deletion or inhibition in accordance with the methods of the present invention.

As used herein 6-phosphogluconate dehydratase (E.C. 4.2.1.12) is an enzyme that catalyses the reaction 6-phospho-D-gluconate↔2-dehydro-3-deoxy-6-phospho-D-gluconate+H₂O. Other names in common use include 6-phosphogluconate dehydratase, 6-phosphogluconic dehydrase, gluconate-6-phosphate dehydratase, gluconate 6-phosphate dehydratase, 6-phosphogluconate dehydrase, and 6-phospho-D-gluconate hydro-lyase.

As used herein 2-keto-3-deoxy-6-phosphogluconate aldolase (E.C. 4.1.2.14), commonly known as KDPG aldolase is an enzyme that catalyses the reaction 2-dehydro-3-deoxy-D-gluconate 6-phosphate ↔pyruvate+D-glyceraldehyde 3-phosphate.

It will be appreciated that any one or more of phosphofructokinase, pyruvate kinase, glycerate mutase, 6-phosphogluconoate dehydratase and 2-keto-3-deoxy-6-phosphogluconate aldolase may be inhibited through contacting the microorganism or host cell of the invention with any agent which reduces or inhibits the levels or activity of the proteins. The inhibition may be direct or indirect. The inhibition may be partial or complete.

The inhibitor is preferably selected from: a small molecule, a peptide, an antibody, an interfering RNA, for example an antisense RNA, microRNA, shRNA, siRNA, that can reduce the activity or levels of one or more of the proteins.

In preferred embodiments, the microorganism or host cell is genetically modified so as to completely delete or partially reduce the expression of one or more of the genes pfkA, pps, gpmA, gpmM, gapA, edd and eda.

The skilled person will be familiar with various techniques for deleting or modifying gene sequences so as to partially or complete reduce gene expression. In certain embodiments, the genetic modification is by use of a CRISPR-Cas9 system. Other genome editing techniques that can be employed include the lambda red recombinase system, random mutagenesis and selection and Multiplex Automated Genome Engineering (MAGE). In one example, a combination of CRISPR-Cas9 and lambda red recombinase may be used, such as outlined in Reisch CR and Prather KL, (2015) The no-SCAR (Scarless Cas9 Assisted Recombineering) system for genome editing in Escherichia coli, Sci Rep. 14(5):15096.

The reduced expression of any one or more of the pfkA, pps, gpmA, gpmM, gapA, edd and eda genes may be a reduced expression of at least 20%, at least 30%, at least 40%, at least 50%, at least 60%, at least 70%, at least 80%, at least 90% or 100% reduced expression.

The gene which is deleted or has reduced expression is preferably pfk or gpmA. In other embodiments, both pfk and gpmA or pfk and gpmM are deleted. Further still, both edd and eda may be deleted. In alternative embodiments, gpmM and/or gpmA in combination with edd and eda are deleted. In still further embodiments, pfk, edd-eda and gpmA or gpmM are deleted.

To further direct oxidation of glucose toward the pentose phosphate pathway, and to maximise the rate of production and yield of hydrogen, the invention also contemplates the increased expression or activity of various endogenous genes (or inhibition of the proteins they encode).

Accordingly, in preferred embodiments, the microorganisms of the present invention are further modified to increase the levels or activity of one or more of the genes encoding phosphoglucomutase, glucose-6-phosphate dehydrogenase, 6-phosphogluconolactonase, 6-glucophosphonate dehydrogenase NAD kinase and soluble pyridine nucleotide transhydrogenase. These proteins are encoded by the genes pgm, zwf, pgl, gnd, yfjB and sthA respectively. Moreover, the methods of the invention include increasing the expression of one or more of the proteins phosphoglucomutase, glucose-6-phosphate dehydrogenase, 6-phosphogluconolactonase, and 6-phosphogluconate dehydrogenase.

As used herein phosphoglucomutase, (PGM) (E.C. 5.3.1.9), also known as glucose-phosphate isomerase, phosphoglucose isomerase/phosphoglucoisomerase (PGI) or phosphohexose isomerase (PHI), is an enzyme that functions as a glycolytic enzyme (glucose-6-phosphate isomerase) that interconverts glucose-6-phosphate (G6P) and fructose-6-phosphate (F6P). Since the reaction is reversible, its direction is determined by G6P and F6P concentrations.

In certain embodiments, level or activity of PGM is increased by increasing the expression of the endogenous pgm gene, for example, by introducing a promoter that enables increased expression of the gene.

In preferred embodiments, the promoter of the pgm gene in E. coli is replaced with the gapA promoter from E. coli. An exemplary gapA promoter (gapAp) 5′-3′ is set forth in SEQ ID NO: 13.

As used herein glucose-6-phosphate dehydrogenase (E.C. 1.1.1.49), also known as G6PD is an enzyme that catalyses the chemical reaction D-glucose 6-phosphate+NADP+ [symbol] 6-phospho-D-glucono-1,5-lactone+NADPH+H+. G6PD converts G6P into 6-phosphoglucono-δ-lactone and is the rate-limiting enzyme of the pentose phosphate pathway. Thus, regulation of G6PD has downstream consequences for the activity of the rest of the pentose phosphate pathway. Glucose-6-phosphate dehydrogenase is stimulated by its substrate G6P. In E. coli, the zwf gene encodes glucose-6-phosphate 1-dehydrogenase. An exemplary amino acid sequence of the E. coli G6PD protein can be found under Uniprot accession POAC53, and exemplary nucleic acid sequences under accession numbers M55005, NP_416366.1, and NC_000913.3. The cognate protein from Zymomonas mobilis is encoded by zwf, an exemplary nucleic acid sequence of which is provided in SEQ ID NO: 16, herein.

In certain embodiments, level or activity of G6PD is increased by increasing the expression of the endogenous zwf gene, for example, by introducing a promoter that enables increased expression of the gene. In certain embodiments, the E. coli zwf promoter is replaced with the osmY promoter (osmYp). An exemplary osmY promoter (osmYp) sequence is set forth in SEQ ID NO: 12. In alternative embodiments, the E. coli zwf promoter is replaced with the E. coli gapA promoter as set forth in SEQ ID NO: 13.

In preferred embodiments, level or activity of G6PD is increased by replacing or supplementing the zwf gene of E. coli with the zwf gene from Zymomonas mobilis. In further embodiments, the zwf gene of E. coli is replaced or supplemented with the zwf gene from any gram negative facultative bacterium.

As used herein 6-phosphogluconolactonase, (E.C. 3.1.1.31), also known as 6PGL or PGLS, is an enzyme that catalyzes the hydrolysis of 6-phosphogluconolactone to 6-phosphogluconic acid (or 6-phospho-D-gluconate+H⁺) in the oxidative phase of the pentose phosphate pathway. 6-phosphogluconolactonase catalyzes the conversion of 6-phosphogluconolactone to 6-phosphogluconic acid, both intermediates in the oxidative phase of the pentose phosphate pathway, in which glucose is converted into ribulose 5-phosphate. The oxidative phase of the pentose phosphate pathway releases CO2 and results in the generation of two equivalents of NADPH from NADP+. The final product, ribulose 5-phosphate, is further processed by the organism during the non-oxidative phase of the pentose phosphate pathway to synthesize biomolecules including nucleotides, ATP, and Coenzyme A. The enzyme that precedes 6PGL in the pentose phosphate pathway, glucose-6-phosphate dehydrogenase, exclusively forms the δ-isomer of 6-phosphogluconolacton. An exemplary E. coli 6PGL sequence can be found under Uniprot accession P52697 and exemplary nucleic acid sequence can be found under accession numbers U27192, NP_415288.1 and NC_000913.3.

In certain embodiments, level or activity of PGL is increased by increasing the expression of the endogenous pgl gene, for example, by introducing a promoter that enables increased expression of the gene.

In preferred embodiments, promoter of the pgl gene in E. coli is replaced with the gapA promoter from E. coli. An exemplary gapA promoter (gapAp) 5′-3′ is set forth in SEQ ID NO: 13.

As used herein glyceraldehyde-3-phosphate dehydrogenase (E.C. 1.2.1.12), also known as GAPDH, and less commonly as G3PDH, is encoded by the gapA gene in E. coli. The protein catalyses the oxidative phosphorylation of glyceraldehyde 3-phosphate (G3P) to 1,3-bisphosphoglycerate (BPG) using the cofactor NAD. The first reaction step involves the formation of a hemiacetal intermediate between G3P and a cysteine residue, and this hemiacetal intermediate is then oxidized to a thioester, with concomitant reduction of NAD to NADH. The reduced NADH is then exchanged with the second NAD, and the thioester is attacked by a nucleophilic inorganic phosphate to produce BPG. An exemplary amino acid sequence of the E. coli GapA protein can be found under Uniprot accession POA9B2, and exemplary nucleic acid sequences under accession numbers X02662, NP_416293.1 and NC_000913.3. The cognate protein from Clostridium acetobutylicum is encoded by gapC, an exemplary nucleic acid sequence of which is provided in SEQ ID NO: 15, herein.

In certain embodiments, level or activity of GAPDH is decreased by decreasing or eliminating the expression of the endogenous gapA gene, by deleting the gene or changing the promoter to reduce the expression of the gene.

In preferred embodiments, the gapA gene in E. coli is replaced with the gapC gene from Clostridium acetobutylicum.

As used herein 6-glucophosphonate dehydrogenase, also referred to as 6-phosphogluconate dehydrogenase, decarboxylating (E.C. 1.1.1.44), is an enzyme that catalyses the oxidative decarboxylation of 6-phosphogluconate to ribulose 5-phosphate and CO₂, with concomitant reduction of NADP to NADPH. In E. coli, 6-glucophosphonate dehydrogenase is encoded by the gnd gene. An exemplary amino acid sequence of the E. coli 6-glucophosphonate dehydrogenase can be found under UniProt accession P00350, and nucleic acid sequences can be found under accession numbers K02072, NP_416533.1 and NC_000913.3. The cognate protein from Corynebacterium glutamicum is encoded by gnd, an exemplary nucleic acid sequence of which is provided in SEQ ID NO: 14, herein.

In certain embodiments, level or activity of 6-glucophosphonate dehydrogenase is increased by increasing the expression of the endogenous gnd gene, for example, by introducing a promoter that enables increased expression of the gene. In certain embodiments, wherein the microorganism is E. coli, the promoter of the gnd gene in E. coli is replaced with the gapA promoter from E. coli. An exemplary gapA promoter (gapAp) 5′-3′ is set forth in SEQ ID NO: 13. In alternative embodiments, the E. coli gnd promoter is replaced with the osmY promoter (osmYp). An exemplary osmY promoter (osmYp) sequence is set forth in SEQ ID NO: 12.

In particularly preferred embodiments, the endogenous promoter for one or more of pgm, zwf, pgl, gnd, tktB or tktA, and talA or talB is replaced with a promoter selected from the group consisting of: the osmY promoter, the gapA promoter, the nirB promoter and the nar promoter.

In preferred embodiments, the gnd gene in from the microorganism (.e.g, E. coli) is replaced with the gnd gene from Corynebacterium glutamicum.

In particularly preferred embodiments, the endogenous promoter for one or more of pgm, zwf, pgl, gnd, tktB or tktA, and talA or talB is replaced with a promoter selected from the group consisting of: the osmY promoter, the gapA promoter, the nirB promoter and the nar promoter. Preferably the osmY, gapA, nirB and/or nar promoters are the endogenous promoters of the organism. More preferably, wherein the microorganism is E. coli, the osmY, gapA, nirB and/or nar promoters are from E. coli.

Sucrose Metabolising Genes

Since most E. coli strains are unable to utilise sucrose as a source of carbon, the microorganisms and methods of the present invention also include modification of the host microorganisms to enable metabolism of sucrose. In certain embodiments, this can be accomplished by modifying the microorganism to express a gene cluster, cscRAKB which have been identified in those strains of E. coli which are able to metabolise sucrose.

Thus, in preferred embodiments, the methods of the invention further comprise providing the host microorganism with a recombinant polynucleotide for enabling expression of nucleic acid sequences encoding sucrose hydrolase (encoded by the cscA gene) and sucrose permease (encoded by the cscB gene). In further embodiments, the methods also comprise providing the microorganism with a recombinant polynucleotide encoding the regulatory proteins CscR and CscK (encoded by the genes cscR and cscK, respectively).

As used herein, sucrose hydrolase refers to the enzyme sucrose-6-phosphate hydrolase, also referred to a sucrose or invertase (E.C. 3.2.1.26) encoded by the cscA gene. An exemplary amino acid sequence of sucrose hydrolase is provided under UniProt accession P40714 and exemplary nucleotide sequences are provided under accession number X81461.

As used herein, sucrose permease refers to the protein encoded by the cscB gene. Sucrose permease is also known as sucrose transport protein and an exemplary amino acid sequence can be found under Uniprot accession number P3000. An exemplary nucleotide sequence encoding sucrose permease can be found under accession X63740 or X81461.

Further still, the E. coli microorganism may be genetically modified to increase the levels or activity of endogenous E. coli phosphoglucomutase (pgm) or xylose isomerase (xylA). The genetic modification may be to increase expression of the endogenous gene (for example, by modification of the promoter region) or by introduction and expression of an exogenous nucleic acid encoding the gene.

In further embodiments, the microorganism is modified to express sucrose phosphorylase from Leuconostoc mesenteroides

Nucleic Acids

An “isolated” nucleic acid molecule is a nucleic acid molecule that is identified and separated from at least one contaminant nucleic acid molecule with which it is ordinarily associated in the natural source of the polypeptide encoding nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express the nucleic acid where, for example, the nucleic acid molecule is in a chromosomal location different from that of natural cells.

The terms “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A nucleic acid sequence which “encodes” a selected polypeptide is a nucleic acid molecule which is transcribed (in the case of DNA) and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

Polynucleotides of the invention can be synthesised according to methods well known in the art, as described by way of example in Sambrook et al (1989, Molecular Cloning—a laboratory manual; Cold Spring Harbor Press).

As used herein, “codon optimised” refers to optimisation of the DNA sequence to resemble the codon usage of genes in host microorganism. In preferred embodiments, the codon usage in the sequence is optimised to resemble that of highly expressed E. coli genes.

The polynucleotide molecules of the present invention may be provided in the form of an expression cassette which includes control sequences operably linked to the inserted sequence, thus allowing for expression of the polypeptide. These expression cassettes, in turn, are typically provided within vectors (e.g., plasmids or recombinant vectors). A suitable vector may be any vector which is capable of carrying a sufficient amount of genetic information, and allowing expression of a polypeptide of the invention.

The present invention thus includes expression vectors that comprise such polynucleotide sequences. Expression vectors are routinely constructed in the art of molecular biology and may for example involve the use of plasmid DNA and appropriate initiators, promoters, enhancers and other elements which may be necessary, and which are positioned in the correct orientation, in order to allow for expression of a desired polypeptide. Other suitable vectors would be apparent to persons skilled in the art. By way of further example in this regard we refer to Sambrook et al.

Thus, a polypeptide of the invention may be provided by delivering such a vector to a cell and allowing transcription from the vector to occur. The skilled person will be familiar with standard techniques for delivery such expression vectors to a cell, including transformation techniques and the like.

The vector may be a plasmid. In certain embodiments, the plasmid is a high copy number plasmid or a low copy number plasmid. Vectors are well known in the art and may include cloning vectors, expression vectors, etc. A cloning vector is a recombinant nucleic acid construct which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is a recombinant nucleic acid construct into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode polypeptides or enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., fluorescent proteins such as green fluorescent protein). Preferred vectors are those capable of autonomous replication and expression of the structural gene products present in the DNA segments to which they are operably joined.

As used herein, a coding sequence and regulatory sequences are said to be “operably” joined or linked when they are covalently linked in such a way as to place the expression or transcription of the coding sequence under the influence or control of the regulatory sequences. If it is desired that the coding sequences be translated into a functional protein, two DNA sequences are said to be operably joined or linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined or linked to a coding sequence if the promoter region were capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene.

Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

The nucleic acids of the present invention are preferably operably linked to promoters such that the subject enzymes are expressed in the cell when cultured under suitable conditions for enabling production of hydrogen, as described herein. The promoters may be specific for individual bacterial cell species. The promoter may be a heterologous promoter which increases the expression of the gene above the typical expression level observed in the cell. The promoter may be an inducible promoter.

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter. It may further be understood that in any embodiment, any of the exemplary expression cassettes, vectors or sequences described herein may be further modified so as to not include a signal peptide sequence.

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, host cell systems and the like. The skilled person is aware that the choice of expression host has ramifications for the type of polypeptide produced.

In some embodiments, the cell is engineered or selected (e.g., as described herein) to produce or have altered, optionally increased, production of a molecule of interest. In some embodiments, the cell comprises a deletion or mutation of one or more genes (e.g., one or more regulatory or competing metabolic genes as described herein). In other examples, the one or more genes that are deleted or mutated are in a competing pathway. Mutations can be single or multiple point mutations, additions, partial internal deletions, N-terminal or C-terminal deletions (truncations), or complete deletions, all of which can affect amino acid sequence encoded the gene(s).

Deletions or mutations can be made using standard methods in the art. Mutations can be non-random, partially random or random, or a combination of these mutations. For example, for a partially random mutation, the mutation(s) may be confined to a certain portion of the nucleic acid molecule encoding a polypeptide in which mutation(s) are to be made.

Culturing and Modification of Microorganisms

In particularly preferred embodiments, culturing of the microorganisms or host cells, as described herein, is performed under aerobic conditions initially to produce biomass then transferred to anaerobic conditions to induce anaerobiosis during expression of the HPGC. The skilled person will be familiar with techniques for creating anaerobiosis, including with the addition of a neutral gas (such as N₂,) or a reductant. However, it will also be appreciated that anaerobiosis can be accomplished simply by culturing the microorganisms in a sealed container in the presence of an oxidisable carbon source.

Further still, culturing of the microorganisms or host cells is preferably performed by inclusion of ferric (iron III) or ferrous (iron II) salts in the culture media. Preferably the ferric (iron III) or ferrous (iron II)salts are provided at a final concentration in the media of at least about 5 μM, at least about 10 μM, at least about 20 μM or at least about 30 μM or more. Preferably the final concentration of ferric (iron III) or ferrous (iron II) salts provided in the culture media is equal to or greater than about 20 μM.

The skilled person will appreciate that culturing of recombinant host cells for production of recombinant proteins will be carried out at a temperature that is optimal for the growth and expression of proteins in the organism. For example, the optimum temperature for growth of E. coli and related bacterial organisms is about 37° C. and the temperature for growth of yeasts for producing recombinant proteins is about 30-32° C. However, the present inventors have found that expression of functional hydrogenase can be further enhanced when using a bacterial expression system, such as E. coli, when the culturing temperature is reduced. Accordingly, in preferred embodiments, where the microorganism or host cell is E. coli, the culturing temperature is no more than about 30° C. The temperature may be between about 10° C. to about 30° C., preferably at least about 15° C. In certain embodiments, the temperature is about 20° C. to about 30° C. In particularly preferred embodiments, the temperature is about 20° C. (for example, 18° C., 19° C., 20° C., 21° C., 22° C.).

“Genetically engineered” or “genetically modified” refers to any cell modified by any recombinant DNA or RNA technology. In other words, the cell has been transfected, transformed, or transduced with a recombinant polynucleotide molecule, and thereby been altered so as to cause the cell to alter expression of a desired protein. Methods and vectors for genetically engineering host cells are well known in the art; for example, various techniques are illustrated in Current Protocols in Molecular Biology, Ausubel et al., eds. (Wiley & Sons, New York, 1988, and quarterly updates). Genetic engineering techniques include but are not limited to expression vectors, targeted homologous recombination, and gene activation (see, for example, U.S. Pat. No. 5,272,071), and trans-activation by engineered transcription factors (see, for example, Segal et al., 1999, Proc Natl Acad Sci USA 96(6):2758-63).

In certain embodiments, the genetic modifications described herein result in an increase in gene expression or function and can be referred to as amplification, overproduction, overexpression, activation, enhancement, addition, or up-regulation of a gene. More specifically, reference to increasing the action (or activity) of enzymes or other proteins discussed herein generally refers to any genetic modification in the microorganism in question that results in increased expression and/or functionality (biological activity) of the enzymes or proteins and includes higher activity of the enzymes (e.g., specific activity or in vivo enzymatic activity), reduced inhibition or degradation of the enzymes, and overexpression of the enzymes. For example, gene copy number can be increased, expression levels can be increased by use of a promoter that gives higher levels of expression than that of the native promoter, or a gene can be altered by genetic engineering or classical mutagenesis to increase the biological activity of an enzyme. Combinations of some of these modifications are also possible.

As used herein, the term “exogenous polynucleotides” is intended to mean polynucleotides that are not derived from naturally occurring polynucleotides in a given organism. Exogenous polynucleotides may be derived from polynucleotides present in a different organism. In accordance with the present invention, an E. coli cell may be genetically modified with a nucleic acid construct which contains one or more exogenous polynucleotides, encoding one or more enzymes which enable the cell to produce hydrogen.

The exogenous polynucleotides may be heterologous or homologous. The term “heterologous” refers to a molecule or activity derived from a source other than the referenced species whereas “homologous” refers to a molecule or activity derived from the host microbial organism. Accordingly, exogenous expression of a nucleic acid molecule of the invention can be through the use of either or both a heterologous or homologous nucleic acid molecule.

The exogenous polynucleotides may be provided in one or more expression constructs (plasmid vectors).

Methods of transforming microorganisms are well known in the art, and can include such non-limiting examples as electroporation, calcium chloride-, or lithium acetate-based methods.

The skilled person will be familiar with methods for confirming successful transformation of relevant constructs, as well as methods for determining whether the transformants possess the relevant enzyme activity provided by the encoded protein. For example, phosphofructokinase activity (and therefore inferring correct protein folding of the encoded protein) can be inferred using a commercially available enzyme assay kit.

Similarly, the skilled person will be familiar with standard techniques to confirm inhibition or deletion of the level of activity of a relevant protein or level of expression of the relevant gene. Successful gene modification, deletion or replacement can be confirmed using standard sequencing techniques. Successful inhibition of protein activity following contacting the cell with an inhibitor can be assessed by assessing for the activity of the relevant protein, for example using a commercially available enzyme assay kit.

The skilled person will also be familiar with general culturing techniques required to induce expression of the polynucleotides in the recombinant microorganism, and thereby induce production of the proteins of the HPGC to produce hydrogen, when required. In some examples, a liquid culture of the recombinant microorganism is grown under anaerobic conditions, supplemented with glucose.

Successful transformation can also be determined by the inclusion of selection marker genes in the plasmid of vector to be transformed into the cell. As used herein, the term “selection marker genes” refer to genetic material that encodes a protein necessary for the survival and/or growth of a host cell grown in a selective culture medium. Typical selection marker genes for use in microorganisms, including in E. coli are well known to the skilled person.

Measurement of hydrogen production can be by any suitable method including as outlined in the Examples. In one simple example, hydrogen production can be gauged simply by observing for the production of bubbles of gas in the culture. In other examples, the production and quantification of hydrogen production is by sampling the gas bubbles and analysing the gas composition by gas chromatography with detection by thermal conductivity or mass spectrometry. In other examples, a Clark-type electrode known to the skilled person may be used, or any other suitable method for detecting hydrogen production.

In any embodiment of the invention, the microorganism, preferably an E. coli microorganism, may be stored for a period of time prior to inducing the production of hydrogen. For example, in certain embodiments, the microorganism of the invention or methods described herein may involve transformation of the microorganism with the required polynucleotides in order to generate a recombinant microorganism capable of generating hydrogen. The microorganism may then be harvested and stored under conditions suitable for storage of the microorganism (for example, at 4° C., −20° C. or −80° C. in a suitable buffer) until required for hydrogen production. It will also be appreciated that the microorganism may be lyophylised until required for further use. Further, it will be understood that the microorganism can be grown under conditions to enable expression of the HPGC and then harvested, where necessary stored, and then resuspended in appropriate solutions supplemented with glucose to initiate bacterial production of hydrogen.

In some examples, the cultured bacteria that have been produced, and that have expressed the HPGC are harvested and fed glucose under isoosmotic conditions to produce hydrogen.

In certain embodiments, the bacteria are encapsulated, for example in calcium alginate beads using standard techniques and are fed glucose in an isosmotic media to produce hydrogen. The skilled person will be familiar with standard manual and mechanism techniques and equipment for bio-encapsulation, including by using a device such as the Inotech Encapsulator IE-50R (EncapBioSystems Inc), or Encapsulator B-390/B-395 pro (Buchi), or related systems. Other methods are described, for example in: Heidebach, et al., (2012) Critical Reviews in Food Science and Nutrition, 52: 291-311; Martin et al., (2015) Innovative Food Science & Emerging Technologies 27:15-25, the entire contents of which are hereby incorporated by reference.

In other examples, the recombinant microorganism does not need to be viable (i.e., capable of reproducing, “growing” or increasing in cell numbers) in order to be able to produce hydrogen in accordance with the present invention. For example, in any embodiment, the methods involve providing or generating a recombinant microorganism as herein described, culturing the microorganism under conditions and for a sufficient time to induce expression of the proteins required for producing hydrogen (e.g., the proteins encoded by the HPGC) and then inactivating the microorganism. Preferably, the inactivated microorganisms remain intact, although it will be understood that this is not an essential requirement.

Inactivated recombinant microorganisms of the invention can be then be used to generate hydrogen, for example as described herein in the Examples.

The skilled person will be familiar with methods for inactivating micrororganisms so that the cells remain intact, but can still be utilised to produce hydrogen (i.e., from the HPGC and other proteins that have been expressed by the cells). Inactivation may be by gamma irradiation or by treatment with an antibiotic (such as mitomycin or similar).

In any embodiment, at least 10%, 20%, 30%, 40%, 50%, 60%, 70%, 80%, 90% or 100% of the microorganisms are inactivated.

Systems and Devices

The present invention also provides systems and devices comprising the microorganisms of the invention, or reactor systems which include methods described herein for producing hydrogen.

In preferred embodiments, the invention further comprises a hydrogen gas collection system for collecting hydrogen gas produced by the microorgansims. A hydrogen gas collection system can be included in the reactor system such that the hydrogen gas generated is collected and is optionally stored for use. Alternatively, the generated hydrogen gas can be directed to a point of use, such as, for example, to a hydrogen fuel powered device.

In some embodiments, a hydrogen gas collection unit includes one or more hydrogen gas conduits for directing a flow of hydrogen gas produced in the reactor system to a storage container or directly to a point of use. In other embodiments, a hydrogen gas conduit is optionally connected to a source of a sweep gas, wherein the hydrogen gas is collected using the sweep gas. An exemplary sweep gas is nitrogen. For example, as hydrogen gas is initially produced, a sweep gas can be introduced into a hydrogen gas conduit, flowing in the direction of a storage container or point of hydrogen gas use. In further embodiments, a hydrogen collection system can include a container for collection of hydrogen from the reactor system. In still other embodiments, a collection system can further include a conduit for passage of hydrogen. The conduit and/or container can be in gas flow communication with a channel provided for outflow of hydrogen gas from the reaction chamber

Fuel cells are electrochemical devices that convert the energy of a fuel directly into electrochemical and thermal energy. Typically, a fuel cell consists of an anode and a cathode, which are electrically connected via an electrolyte. A fuel such as, for example, hydrogen, is fed to the anode where it is oxidized with the help of an electrocatalyst. At the cathode, the reduction of an oxidant such as oxygen (or air) takes place. The electrochemical reactions which occur at the electrodes produce a current and thereby electrical energy. Commonly, thermal energy is also produced which may be harnessed to provide additional electricity or for other purposes. Currently, the most common electrochemical reaction for use in a fuel cell is that between hydrogen and oxygen to produce water. Molecular hydrogen itself can be fed to the anode where it is oxidized, and the electrons produced are passed through an external circuit to the cathode where oxidant is reduced. Ion flow through an intermediate electrolyte maintains charge neutrality.

The fuel cells of the present subject matter utilize hydrogen as a fuel wherein the source of hydrogen is from the recombinant microorganisms of the present subject matter.

Typically, hydrogen is present in the fuel source in an amount of at least about 2% by volume, preferably at least about 5% and more preferably at least about 10% by volume, for example about 25%, 50%, 75% or 90% by volume. Where an inert gas is used to form part of the fuel gas, the inert gas is typically present in an amount of at least about 10%, such as at least about 25%, 50% or 75% by volume, most preferably at least about 80% by volume.

Generally, the fuel source is supplied from an optionally pressurized container of the fuel source in gaseous or liquid form. The fuel source is supplied to the electrode via an inlet, which can optionally comprise a valve. An outlet is also provided which enables used or waste fuel source to leave the fuel cell.

The oxidant typically includes oxygen, although any other suitable oxidant can be used. The oxidant source typically provides the oxidant to the cathode in the form of a gas which includes the oxidant, hi some embodiments, the oxidant can be provided in liquid form. Generally, the oxidant source also includes an inert gas, although the oxidant in its pure form can also be used. For example, a mixture of oxygen with one or more gases such as nitrogen, helium, neon or argon can be used. The oxidant source can optionally comprise further components, for example alternative oxidants or other additives. An example of a suitable oxidant source is air.

Typically, oxygen is present in the oxidant source in an amount of at least about 2% by volume, preferably at least about 5% and more preferably at least about 10% by volume.

Generally, the oxidant source is supplied from an optionally pressurized container of. the oxidant source in gaseous or liquid form. The oxidant source is supplied to the electrode via an inlet, which optionally comprises a valve. An outlet is also provided which enables used or waste oxidant source to leave the fuel cell.

The anode can be made of any conducting material for example stainless steel, brass or carbon, which can be graphite. The surface of the anode can, at least in part, be coated with a different material which facilitates adsorption of the catalyst. The surface onto which the catalyst is adsorbed is of a material which does not cause the hydrogenase to denature. Suitable surface materials include graphite, such as, for example, a polished graphite surface or a material having a high surface area such as carbon cloth or carbon sponge. Materials with a rough surface and/or with a high surface area are generally preferred.

The cathode can be made of any suitable conducting material which will enable an oxidant to be reduced at its surface. For example materials used to form the cathode in conventional fuel cells can be used. An electrocatalyst can, if desired, be present at the cathode. This electrocatalyst can, for example, be coated or adsorbed on the cathode itself, or it can be present in a solution surrounding the cathode. Suitable electrocatalysts include those used in conventional fuel cells such as platinum. Biological catalysts can also be used for this purpose, and in particular, the combination of enzymes and accessory proteins described herein.

The fuel cell of the present subject matter is typically operated at a temperature of at least about 25° C., more preferably at least about 30° C. It is preferred that the fuel cell is operated at a temperature of from about 35° C. to about 65° C., such as from about 40° C. to about 50° C. A higher temperature increases the rate of reaction and leads to a higher oxidation current.

A fuel cell, as described above, can be operated under the conditions described above, to produce a current in an electrical circuit. The fuel cell is operated by supplying hydrogen to the anode and supplying an oxidant to the cathode. The fuel cell of the invention is capable of producing current densities of at least about 0.5 mA, typically at least about 0.8 mA, 1 mA or 1.5 mA per cm2 of surface area of the positive electrode. For example, the fuel cell of the invention can produce a current of at least about 2 mA, such as at least about 3 mA per cm2 of surface area of the positive electrode.

### EXAMPLES

### Example 1

Materials and Methods

Bacterial Strains and Plasmids

Wild-type (DH5a) Escherichia coli NEB 5-alpha (CP017100.1, (Anton and Raleigh, 2016)), an immediate fhuA2 derivative of DH5a and derivative of K-12, was purchased from New England Biolabs and maintained on plates containing Luria-Bertani (LB) medium and 1.5% agar. Antibiotics chloramphenicol (Cam) 30 mg L⁻¹ and kanamycin sulfate (Kan) 50 mg L⁻¹ were included as required. Mutant strains of DH5a were constructed using CRISPR/Cas9 methods (Reisch and Prather, 2015). Plasmid pHPGC (Cam^{R}) was constructed using standard Biobrick assembly methods, restriction digest and ligation. The resulting plasmid was transformed into WT and mutant strains by standard procedures. Strains and plasmids used for hydrogen production are listed in Table 2. The sequences of the various components of the HPGC and heterologous promoter and gene sequences are given in Table 1. The genome sequences of wild type and mutant strains used for hydrogen production were confirmed using Nanopore Sequencing technology.

Fermentation

Pre-cultures in 100 mL Super Optimal broth with Catabolite repression (SOC) media containing Cam were inoculated with single colonies of E. coli DH5a, DH5a-HPGC, ΔpfkA-HPGC or ΔgpmA-HPGC (using the HPGC constructs specified in SEQ ID NO: 10 and 30-40). The pre-cultures were incubated overnight at 37° C. to approximately OD₆₀₀ 2 (1.6×10⁹ cells) and inoculated into 2 L of SOC media (pH 7) with the addition of sterile filtered (0.2 μm) 20 mM D-glucose, 1 mM iron sulfate and Cam (30 mg L⁻¹).

Cells were grown anaerobically to OD₆₀₀ 0.6 (4.8×10⁸ cells) at 100 rpm agitation speed with temperature being controlled at 37° C. and pH maintained at 7 by titrating 1 M sodium hydroxide with the aid of a fermenter (Eppendorf, BioFlow 120 and BioFlo®/CelliGen® 115 Fermenter/Bioreactor). When cell growth reached OD₆₀₀ 0.6 (4.8×10⁸ cells), the culture temperature was reduced to 18° C. prior to induction. The cells were induced with sterile filtered Isopropyl-b-D-thiogalactopyranoside (IPTG, 1 mM) and 1 mM iron sulfate. Fermentation parameters during induction were the same as above and cells were grown overnight to approximately OD₆₀₀ 2 (1.6×10⁹ cells). Following, cells were harvested by centrifugation, 4650 rcf for 15 minutes at 18° C. The cell pellets were washed three times in 1× phosphate-buffered saline (PBS) pH 7.4 (10 mM) or 1× PBS (approximately 50 mM) pH 8.0 containing 1 mM sodium dithionite and stored at 4° C.

Biohydrogen Reactor

Cell pellets of DH5a, DH5a-HGPC, ΔpfkA-HGPC or ΔgpmA-HGPC obtained through fermentation were resuspended in 1× PBS (10 mM) pH 7.4 or 1× PBS (approximately 50 mM) pH 8.0 at 20 OD₆₀₀ with optionally 1 mM dithionite (final volume 50 mL) and placed into a 100 mL side-arm conical flask to test and measure hydrogen production. The flask was sealed with a rubber stopper with a pH probe protruding into the suspended cells. The side-arm of the flask was connected to a custom-made apparatus designed to measure gas volume. A Teflon coated magnetic bar was placed inside the conical flask and the flask was placed on a magnetic stirrer plate. The headspace of the conical flask was purged with three volumes of 100% nitrogen gas prior to the addition of D-glucose (final concentration of 20 mM) to initiate bacterial hydrogen formation. The experiment was performed at room temperature of approximately 22° C.

Hydrogen, Carbon Dioxide and pH Measurements

Headspace gas samples (5 μL) and pH measurements were taken and recorded, respectively, from the biohydrogen reactor at the start (immediately following D-glucose addition) and approximately every 15-20 minutes until gas production has almost ceased. The gas samples were analysed using Shimadzu Nexis, GC-2030 with column (Restek, ShinCarbon ST Micropacked GC Column, Cat. #19808) and GC method: SPL1 temperature 100° C., column flow 6 mL·min-1, DTCD temperature 180° C., oven temperature 40° C. held for 3 minutes, then to 170° C. at 15° C.min-1, hold for 2 minutes at 170° C. Carrier gas was Argon. Column specifications, ShinCarbon ST, 100/120 mesh, 2 m, 1/16 in. OD, 1.0 mm).

Gas standards (20% hydrogen, 20% nitrogen, 20% carbon monoxide and 20% carbon dioxide [product number: PGS402470D]; 10% hydrogen, 10% nitrogen, 10% carbon monoxide and 10% carbon dioxide with argon balance gas [product number: PGS402469D]; and 50% oxygen with argon balance gas [product number: PGS402471 D2]) were used to determine the % concentration of hydrogen and carbon dioxide. Oxygen and nitrogen gas were also measured to monitor air leaks into the conical side-arm flask during the experiment. Gas standards were supplied by BOC Australia.

NMR Analysis

For each sample, 700 μL of cell culture was pelleted by centrifugation at 20,018 rcf for 2 minutes. The supernatant was collected (600 μL) into 15-mL Falcon tubes and then frozen at −80 μL). The samples were then freeze-dried and resuspended in deuterium oxide (800 μL). Resuspension was placed into NMR tubes (Norell Sample Vault Series, standard wall, closed cap, parameter 700 MHz frequency, diam.×L 5 mm×178 mm, mfr no. Norell, SVCP-5-178-96PK). All NMR spectra were recorded at 298K on a Bruker AVIIIHD 400 MHz NMR Spectrometer equipped with a 5 mm BBFO SmartProbe. Spectra were processed and analysed using Topspin 3.5. 1H spectra were recorded with a spectral width of 8013 Hz (20.0 ppm) over 64K data points.

Results

The DH5a-HPGC, ΔpfkA-HPGC, ΔgpmA-HPGC or DH5a-H₁-HEGF (which is HPGC lacking petF and FNR) strains of E. coli produce significant quantities of gas within a 2-hour period after the addition of glucose. The cessation of hydrogen production correlates with the complete consumption of glucose. ΔgpmA-HGPC (e.g., SEQ ID NO: 10) produced 0.95 moles of hydrogen per mole of glucose; ΔpfkA-HGPC produced 0.85 moles of hydrogen per mole of glucose; DH5a-HGPC produced 0.45 moles of hydrogen per mole of glucose, DH5a-H1-HEFG produced 0.45 moles of hydrogen per mole of glucose.

Maximum rates of hydrogen gas production are similar for the HPGC containing strains at 22° C.; being 3.6+/−0.06 L of hydrogen gas per L cells at 200 OD₆₀₀ per hour. Rates were lower for those strains lacking HPGC; with no detectable hydrogen under these conditions for DH5a and DH5a-H1-HEFG giving ˜1.2 L of hydrogen gas per L cells at 200 OD600 per hour, which lacked the petF-FNR.

If not buffered sufficiently the hydrogen production ceases when the pH falls below 5. The drop in pH is due to production of organic acids lactate, succinate, pyruvate and acetate, and the ΔpfkA and ΔgpmA mutants have reduced production of organic acids (FIG. 2).

### Example 2: Rate of Hydrogen Production by Targeting Lower Section of Glycolytic Pathway

Evidence that reducing the flow of carbon from glucose through the lower section of the glycolytic pathway, is shown in FIG. 4. Deletion of the genes gpmM or gpmA improves the rate of hydrogen production.

Increasing expression of gnd and zwf also unexpectedly improved the rate of hydrogen production as shown in FIG. 4.

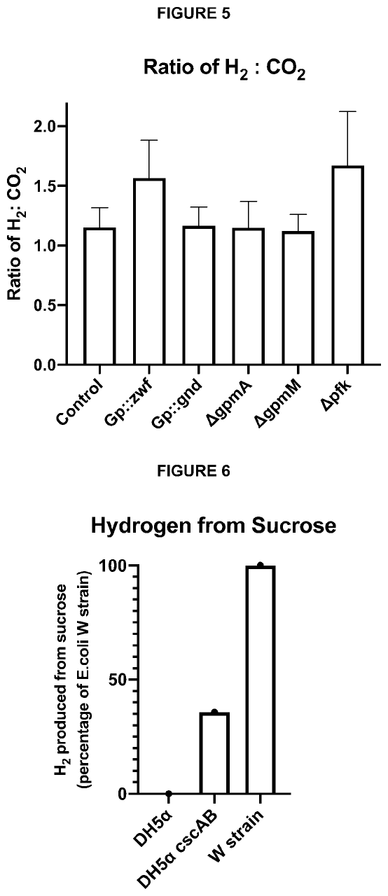

FIG. 5 also provides evidence that increasing the flux of carbon from glucose through the Pentose Phosphate pathway (PPP) increases the ratio of H₂ to CO₂. The theoretical maximum ratio under anaerobic conditions is 2:1 if all the carbons in glucose are metabolised to CO₂ via the pentose phosphate pathway and the reductants produced are used to make H₂. If the glucose goes through glycolysis then the ratio is 1:1 under anaerobic conditions. Increasing the flux through the PPP will improve the overall yield of H₂ made from glucose.

An increase in metabolism through the PPP can be achieved by increasing activity and/or expression of zwf and/or gnd. Alternatively this can also be achieved by reducing the flux from the section of glycolysis with intermediates having 6 carbons to the section of glycolysis with intermediates having 3 carbons by reducing activity of pfk (as for Δpfk). Data shown in FIG. 5 shows increasing expression of zwf or reducing pfk activity increases the ratio of hydrogen to CO₂ and hence flux through the pentose phosphate pathway. As gnd encodes an enzyme that is downstream in the metabolic pathway compared to the enzyme encoded by zwf, the increase in activity of gnd in this mutant has no significant effect on the ratio H₂ to CO₂ ratio compared to the wt DH5a.

TABLE 1
Sequence information

| SEQ | | |
|---|---|---|
| ID NO: | Description | Nucleic acid or amino acid sequence |
| 1 | Promoter for hydEF gene | CTAGAGGGCTTTACACTTTATGCTTCCGGCTCGTATGTTGT |
| | | GTGGA |
| 2 | hydEF nucleic acid | AAGAAGGAGATATACATATGGCTCATAGTTTAAGCGCACAT |
| | sequence encoding HydEF | TCCCGTCAGGCCGGAGATCGCAAACTGGGCGCAGGTGCG |
| | | GCATCTAGCCGCCCATCATGTCCTTCTCGCCGCATTGTCC |
| | | GCGTGGCGGCCCATGCTTCTGCGTCCAAGGCGACTCCCG |
| | | ATGTACCTGTTGACGATCTTCCTCCTGCGCATGCCCGTGCA |
| | | GCAGTAGCGGCCGCTAACCGTCGTGCGCGCGCTATGGCA |
| | | TCAGCGGAGGCCGCGGCAGAGACCCTGGGCGATTTCCTG |
| | | GGCTTAGGGAAGGGGGGGCTTTCGCCGGGCGCAACCGCC |
| | | AACCTTGATCGTGAGCAAGTATTAGGTGTGTTGGAGGCGG |
| | | TCTGGCGTCGTGGAGACCTTAATCTGGAGCGTGCGTTGTA |
| | | CAGCCATGCCAATGCGGTGACTAACAAATACTGTGGTGGG |
| | | GGGGTCTACTATCGCGGCCTTGTGGAGTTCAGTAATATTTG |
| | | CCAAAACGATTGCTCATATTGTGGGATTCGCAACAATCAAA |
| | | AAGAAGTTTGGCGCTATACTATGCCGGTTGAAGAGGTGGT |
| | | CGAGGTAGCTAAGTGGGCGCTTGAAAACGGCATTCGCAAC |
| | | ATTATGTTGCAAGGGGGTGAATTAAAGACAGAGCAACGCTT |
| | | AGCCTACTTGGAGGCATGCGTCCGTGCGATTCGCGAGGAG |
| | | ACAACGCAGTTAGACCTTGAAATGCGCGCACGTGCCGCGA |
| | | GTACGACAACAGCGGAAGCGGCAGCCAGTGCACAGGCAG |
| | | ACGCCGAAGCTAAACGCGGCGAGCCAGAATTGGGTGTGGT |
| | | CGTATCCTTAAGCGTCGGAGAACTTCCTATGGAACAGTATG |
| | | AGCGTTTGTTCCGCGCCGGCGCCCGTCGCTATCTGATCCG |
| | | CATTGAAACCTCGAATCCCGACCTGTACGCGGCACTTCAC |
| | | CCGGAGCCGATGTCCTGGCACGCGCGTGTAGAATGCCTG |
| | | CGCAACCTGAAGAAAGCTGGGTATATGTTGGGCACAGGAG |
| | | TGATGGTCGGCTTGCCGGGACAAACCTTACACGACCTGGC |
| | | TGGGGATGTCATGTTCTTTCGCGACATTAAAGCGGACATGA |
| | | TCGGTATGGGCCCCTTCATTACGCAGCCTGGGACGCCTGC |
| | | AACTGATAAGTGGACCGCGCTGTATCCGAACGCCAATAAG |
| | | AACAGCCACATGAAATCTATGTTCGATCTGACCACTGCTAT |
| | | GAATGCACTTGTACGTATTACGATGGGGAACGTAAATATCA |
| | | GTGCTACGACTGCATTACAAGCGATTATCCCCACTGGACGT |
| | | GAAATTGCGCTTGAGCGCGGCGCAAATGTTGTCATGCCTA |
| | | TTTTAACGCCTACTCAGTATCGCGAGTCCTATCAGTTATATG |
| | | AGGGTAAGCCCTGCATTACGGATACTGCTGTTCAGTGTCGT |
| | | CGTTGCCTGGACATGCGTTTGCACTCCGTTGGCAAAACGT |
| | | CTGCGGCCGGAGTTTGGGGCGATCCTGCTTCGTTCTTGCA |
| | | TCCCATCGTTGGCGTCCCAGTCCCGCACGACTTGTCATCA |
| | | CCTGCTTTGGCCGCTGCTGCAAGTGCGGATTTTCACGAGG |
| | | TTGGCGCAGGTCCCTGGAACCCCATCCGACTAGAGCGTCT |
| | | GGTTGAAGTGCCGGACCGTTACCCTGACCCCGATAACCAC |
| | | GGACGCAAAAAGGCAGGAGCCGGGAAAGGAGGCAAGGCG |
| | | CATGACTCTCACGACGATGGCGATCACGACGACCACCACC |
| | | ACCACCACGGAGCTGCCCCCGCGGGTGCAGCGGCTGGAA |
| | | AGGGTACCGGTGCAGCTGCAATTGGTGGCGGAGCGGGGG |
| | | CTAGCCGTCAACGCGTAGCAGGGGCTGCTGCCGCCTCTG |
| | | CTCGTCTGTGTGCGGGAGCTCGCCGCGCTGGCCGTGTTGT |
| | | CGCGTCGCCATTGCGCCCAGCTGCCGCTTGCCGTGGTGT |
| | | GGCCGTCAAGGCCGCAGCTGCTGCTGCGGGTGAAGACGC |
| | | TGGTGCTGGTACGTCTGGCGTAGGTTCAAATATTGTTACGT |
| | | CTCCCGGAATCGCTAGCACTACGGCACATGGTGTTCCTCG |
| | | TATCAATATTGGGGTGTTCGGCGTGATGAATGCAGGAAAGT |
| | | CTACACTTGTGAATGCTCTGGCGCAACAGGAAGCATGCATT |
| | | GTTGACTCAACCCCCGGCACGACCGCAGATGTAAAAACAG |
| | | TTTTGCTTGAGCTTCATGCCCTTGGACCAGCCAAGTTGCTG |
| | | GACACAGCCGGATTAGACGAAGTCGGTGGACTTGGGGATA |
| | | AAAAGCGCCGTAAAGCCCTGAATACGCTGAAGGAGTGCGA |
| | | TGTTGCTGTGCTGGTTGTTGACACTGATACGGCAGCCGCC |
| | | GCAATCAAATCGGGACGCCTTGCTGAAGCCCTGGAATGGG |
| | | AATCGAAAGTAATGGAACAGGCACACAAGTATAATGTCAGT |
| | | CCTGTACTGCTTCTGAATGTAAAATCACGCGGGCTGCCTGA |
| | | AGCGCAGGCAGCTAGCATGCTTGAGGCGGTCGCTGGTATG |
| | | CTTGACCCATCCAAGCAAATCCCTCGTATGTCGCTGGATTT |
| | | AGCGTCCACCCCCCTGCACGAGCGTAGTACGATTACGTCT |
| | | GCATTCGTCAAGGAAGGAGCAGTGCGCAGTTCACGCTATG |
| | | GGGCTCCTCTGCCGGGGTGTTTGCCCCGTTGGTCTCTTGG |
| | | ACGCAACGCCCGCTTACTGATGGTAATCCCGATGGATGCC |
| | | GAAACCCCAGGTGGACGTCTTCTTCGTCCTCAAGCGCAAG |
| | | TTATGGAGGAAGCAATCCGCCATTGGGCTACCGTGCTTTC |
| | | CGTACGCCTGGATTTAGACGCCGCACGTGGCAAATTGGGG |
| | | CCCGAGGCCTGTGAGATGGAGCGTCAGCGTTTCGACGGG |
| | | GTAATTGCTATGATGGAACGTAATGACGGACCCACCCTGGT |
| | | GGTGACAGATTCTCAAGCGATCGATGTTGTACACCCTTGGA |
| | | CCCTGGACCGCTCATCAGGACGTCCGTTGGTTCCAATTACT |
| | | ACCTTTAGCATCGCTATGGCGTACCAACAAAACGGCGGAC |
| | | GTTTGGACCCGTTTGTGGAAGGATTGGAAGCATTGGAGAC |
| | | GTTACAAGATGGGGATCGCGTTCTTATTTCGGAAGCGTGTA |
| | | ATCATAACCGTATCACCTCCGCTTGCAACGACATTGGAATG |
| | | GTGCAGATCCCTAACAAGCTGGAGGCAGCACTTGGCGGGA |
| | | AAAAGTTACAAATCGAACATGCGTTTGGTCGCGAGTTTCCC |
| | | GAGCTTGAGAGTGGGGGTATGGATGGATTGAAGTTAGCGA |
| | | TCCATTGTGGAGGGTGTATGATCGACGCTCAGAAAATGCA |
| | | GCAGCGTATGAAAGACTTGCATGAGGCTGGGGTGCCGGTC |
| | | ACTAATTATGGGGTGTTTTTCTCTTGGGCAGCTTGGCCCGA |
| | | CGCCCTTCGTCGCGCATTGGAACCGTGGGGAGTGGAGCC |
| | | ACCGGTAGGTACTCCTGCTACCCCCGCAGCCGCGCCTGCT |
| | | ACGGCAGCGTCCGGGGTATAATACTA |
| 3 | Promoter for hydG gene | CTAGAGGGCTTTACACTTTATGCTTCCGGCTCGTATGTTGT |
| | | GTGGA |
| 4 | Nucleic acid sequence | AAGAAGGAGATATACATATGTCGGTCCCCCTACAGTGCAAT |
| | encoding HydG | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 5 | Promoter for hydA | CTAGAGGGCTTTACACTTTATGCTTCCGGCTCGTATGTTGT |
| | | GTGGA |
| 6 | Nucleic acid sequence | AAGAAGGAGATATACATATGTCCGCATTAGTACTTAAGCCC |
| | encoding HydA (Hyd1) | TGTGCGGCAGTATCCATCCGTGGCTCCTCGTGTCGCGCAC |
| | | GCCAAGTAGCCCCACGCGCTCCGCTTGCAGCTAGCACGGT |
| | | TCGTGTCGCTCTTGCAACCCTGGAGGCACCAGCGCGTCGT |
| | | TTAGGAAACGTCGCCTGTGCCGCAGCGGCACCGGCCGCA |
| | | GAGGCACCCTTGTCTCACGTCCAGCAAGCACTGGCCGAAC |
| | | TGGCAAAGCCCAAAGATGACCCCACGCGTAAGCACGTTTG |
| | | CGTTCAAGTCGCTCCCGCAGTGCGTGTCGCTATTGCTGAA |
| | | ACCTTAGGGCTTGCGCCGGGCGCTACGACACCGAAACAAT |
| | | TAGCAGAAGGCCTGCGTCGCTTGGGCTTTGACGAGGTTTT |
| | | CGATACGCTGTTCGGGGCCGACTTGACGATCATGGAAGAG |
| | | GGCTCAGAACTTTTGCACCGTCTGACGGAGCACTTGGAAG |
| | | CACACCCGCACTCTGATGAGCCGCTGCCTATGTTTACCAG |
| | | CTGCTGTCCTGGTTGGATCGCGATGCTGGAGAAATCATAC |
| | | CCAGACCTTATCCCTTATGTAAGTTCTTGCAAATCCCCTCAA |
| | | ATGATGCTGGCTGCTATGGTCAAATCGTATCTGGCGGAAAA |
| | | AAAGGGGATTGCACCTAAAGATATGGTTATGGTAAGCATTA |
| | | TGCCATGCACACGCAAGCAGAGTGAGGCGGATCGTGATTG |
| | | GTTTTGTGTCGACGCGGACCCAACACTTCGCCAATTGGAC |
| | | CATGTGATCACGACCGTAGAGCTGGGGAATATTTTCAAAGA |
| | | GCGTGGGATCAACCTTGCGGAATTACCCGAGGGGGAGTG |
| | | GGACAACCCAATGGGGGTAGGCTCCGGTGCTGGAGTACTT |
| | | TTTGGCACCACTGGAGGGGTAATGGAGGCGGCGCTGCGT |
| | | ACTGCATACGAATTATTTACTGGAACCCCCTTACCCCGCCT |
| | | TTCGCTTTCGGAGGTGCGCGGCATGGATGGCATTAAAGAG |
| | | ACTAACATCACGATGGTACCGGCCCCTGGCAGCAAGTTTG |
| | | AAGAACTGTTAAAGCACCGCGCGGCAGCGCGTGCCGAGG |
| | | CTGCTGCACACGGAACACCTGGTCCCTTGGCCTGGGACGG |
| | | CGGCGCAGGGTTCACCTCGGAGGACGGGCGTGGGGGTAT |
| | | CACTCTTCGTGTGGCGGTAGCTAATGGCTTGGGAAACGCC |
| | | AAAAAGTTGATCACAAAGATGCAGGCGGGAGAGGCGAAGT |
| | | ATGACTTTGTAGAAATCATGGCGTGTCCAGCGGGATGCGTT |
| | | GGGGGCGGCGGGCAGCCACGTTCCACCGATAAAGCAATT |
| | | ACCCAGAAGCGCCAGGCTGCACTTTACAATCTGGACGAGA |
| | | AGTCGACTCTGCGCCGCTCCCATGAAAACCCGTCTATCCG |
| | | TGAGTTATACGACACTTATTTGGGTGAGCCCTTAGGGCACA |
| | | AAGCACACGAACTTTTACATACTCACTATGTAGCTGGCGGG |
| | | GTCGAGGAGAAGGATGAGAAAAAGTGACCAGGCATCAAAT |
| | | AAAACGAAAGGCTCAGTCGAAAGACTGGGCCTTTCGTTTTA |
| | | TCTGTTGTTTGTCGGTGAACGCTCTCTACTAGAGTCACACT |
| | | GGCTCACCTTCGGGTGGGCCTTTCTGCGTTTATATA |
| 7 | Promoter for Ferrodoxin- | CTAGAGGGCTTTACACTTTATGCTTCCGGCTCGTATGTTGT |
| | FNR | GTGGA |
| 8 | Nucleic acid sequence | AAGAAGGAGATATACATATGGCGATGCGTTCCACATTTGCT |
| | encoding Ferredoxin | GCGCGTGTCGGTGCCAAACCGGCAGTGCGTGGGGCGCGT |
| | | CCCGCGTCGCGTATGTCTTGCATGGCCTATAAGGTTACACT |
| | | GAAAACGCCATCAGGCGATAAAACAATCGAATGTCCGGCG |
| | | GACACCTATATCCTGGATGCTGCGGAGGAAGCCGGGCTGG |
| | | ACCTTCCCTATAGTTGCCGTGCGGGCGCCTGTTCATCCTGT |
| | | GCTGGCAAAGTGGCAGCTGGAACGGTAGATCAATCTGATC |
| | | AGTCCTTTCTGGATGACGCTCAGATGGGGAACGGATTCGT |
| | | CCTGACATGTGTCGCGTATCCTACAAGCGACTGCACTATCC |
| | | AAACTCACCAAGAGGAGGCTTTATATTAA |
| 9 | Nucleic acid sequence | AAGAAGGAGATATACATATGCAAACTGTTCGCGCTCCAGCA |
| | encoding FNR | GCTTCAGGTGTTGCCACACGTGTCGCAGGTCGTCGTATGT |
| | | GTCGTCCGGTTGCGGCGACGAAGGCTTCCACGGCTGTTAC |
| | | CACAGACATGTCGAAGCGCACTGTTCCAACCAAGTTAGAG |
| | | GAAGGTGAAATGCCGTTGAACACGTACAGTAATAAAGCTCC |
| | | GTTCAAGGCAAAGGTTCGTTCCGTGGAAAAAATCACAGGA |
| | | CCAAAAGCCACAGGTGAGACGTGCCACATCATTATTGAAAC |
| | | CGAGGGGAAGATCCCGTTTTGGGAGGGACAATCGTACGGT |
| | | GTAATTCCGCCGGGGACCAAGATCAACTCTAAGGGAAAAG |
| | | AAGTGCCTCATGGTACTCGTCTTTATTCGATTGCTTCTAGTC |
| | | GTTACGGAGATGACTTCGATGGTCAAACGGCATCGCTGTG |
| | | TGTTCGCCGCGCGGTATACGTCGATCCAGAGACTGGAAAG |
| | | GAGGACCCCGCGAAAAAAGGACTGTGTAGTAACTTTTTGTG |
| | | TGATGCCACACCAGGCACGGAAATTTCCATGACAGGGCCC |
| | | ACAGGAAAAGTATTGCTTCTGCCAGCAGACGCGAACGCGC |
| | | CATTAATCTGTGTCGCAACGGGGACTGGAATCGCGCCTTTT |
| | | CGCTCATTCTGGCGCCGTTGCTTCATCGAGAATGTCCCAA |
| | | GTTATAAGTTCACTGGCCTTTTCTGGTTGTTTATGGGTGTC |
| | | GCTAACTCTGATGCTAAATTGTACGATGAGGAACTGCAAGC |
| | | TATCGCAAAAGCGTATCCGGGACAATTCCGCCTGGACTAT |
| | | GCCTTGTCTCGTGAACAAAATAATCGCAAGGGCGGGAAAA |
| | | TGTACATCCAAGATAAGGTTGAAGAGTATGCCGACGAAATT |
| | | TTCGATTTATTGGATAATGGGGCGCACATGTACTTCTGCGG |
| | | GTTAAAGGGGATGATGCCAGGCATCCAAGATATGTTAGAAC |
| | | GCGTTGCAAAAGAAAAGGGGCTGAACTACGAAGAGTGGGT |
| | | CGAGGGGTTAAAGCACAAGAATCAATGGCATGTTGAAGTCT |
| | | ACTAACCAGGCATCAAATAAAACGAAAGGCTCAGTCGAAAG |
| | | ACTGGGCCTTTCGTTTTATCTGTTGTTTGTCGGTGAACGCT |
| | | CTCTACTAGAGTCACACTGGCTCACCTTCGGGTGGGCCTTT |
| | | CTGCGTTTATATA |
| 10 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | HydA from Chlamydomonas | TTGCTGCGCGTGTCGGTGCCAAACCGGCAGTGCGTGGGG |
| | reinhardtii | CGCGTCCCGCGTCGCGTATGTCTTGCATGGCCTATAAGGT |
| | | TACACTGAAAACGCCATCAGGCGATAAAACAATCGAATGTC |
| | | CGGCGGACACCTATATCCTGGATGCTGCGGAGGAAGCCG |
| | | GGCTGGACCTTCCCTATAGTTGCCGTGCGGGCGCCTGTTC |
| | | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATACTAGAGGGCTTTACACTTTATGCTTCCGGC |
| | | TCGTATGTTGTGTGGAAAGAAGGAGATATACATATGTCCGC |
| | | ATTAGTACTTAAGCCCTGTGCGGCAGTATCCATCCGTGGCT |
| | | CCTCGTGTCGCGCACGCCAAGTAGCCCCACGCGCTCCGCT |
| | | TGCAGCTAGCACGGTTCGTGTCGCTCTTGCAACCCTGGAG |
| | | GCACCAGCGCGTCGTTTAGGAAACGTCGCCTGTGCCGCAG |
| | | CGGCACCGGCCGCAGAGGCACCCTTGTCTCACGTCCAGC |
| | | AAGCACTGGCCGAACTGGCAAAGCCCAAAGATGACCCCAC |
| | | GCGTAAGCACGTTTGCGTTCAAGTCGCTCCCGCAGTGCGT |
| | | GTCGCTATTGCTGAAACCTTAGGGCTTGCGCCGGGCGCTA |
| | | CGACACCGAAACAATTAGCAGAAGGCCTGCGTCGCTTGGG |
| | | CTTTGACGAGGTTTTCGATACGCTGTTCGGGGCCGACTTG |
| | | ACGATCATGGAAGAGGGCTCAGAACTTTTGCACCGTCTGA |
| | | CGGAGCACTTGGAAGCACACCCGCACTCTGATGAGCCGCT |
| | | GCCTATGTTTACCAGCTGCTGTCCTGGTTGGATCGCGATG |
| | | CTGGAGAAATCATACCCAGACCTTATCCCTTATGTAAGTTC |
| | | TTGCAAATCCCCTCAAATGATGCTGGCTGCTATGGTCAAAT |
| | | CGTATCTGGCGGAAAAAAAGGGGATTGCACCTAAAGATAT |
| | | GGTTATGGTAAGCATTATGCCATGCACACGCAAGCAGAGT |
| | | GAGGCGGATCGTGATTGGTTTTGTGTCGACGCGGACCCAA |
| | | CACTTCGCCAATTGGACCATGTGATCACGACCGTAGAGCT |
| | | GGGGAATATTTTCAAAGAGCGTGGGATCAACCTTGCGGAA |
| | | TTACCCGAGGGGGAGTGGGACAACCCAATGGGGGTAGGC |
| | | TCCGGTGCTGGAGTACTTTTTGGCACCACTGGAGGGGTAA |
| | | TGGAGGCGGCGCTGCGTACTGCATACGAATTATTTACTGG |
| | | AACCCCCTTACCCCGCCTTTCGCTTTCGGAGGTGCGCGGC |
| | | ATGGATGGCATTAAAGAGACTAACATCACGATGGTACCGG |
| | | CCCCTGGCAGCAAGTTTGAAGAACTGTTAAAGCACCGCGC |
| | | GGCAGCGCGTGCCGAGGCTGCTGCACACGGAACACCTGG |
| | | TCCCTTGGCCTGGGACGGCGGCGCAGGGTTCACCTCGGA |
| | | GGACGGGCGTGGGGGTATCACTCTTCGTGTGGCGGTAGC |
| | | TAATGGCTTGGGAAACGCCAAAAAGTTGATCACAAAGATGC |
| | | AGGCGGGAGAGGCGAAGTATGACTTTGTAGAAATCATGGC |
| | | GTGTCCAGCGGGATGCGTTGGGGGCGGCGGGCAGCCACG |
| | | TTCCACCGATAAAGCAATTACCCAGAAGCGCCAGGCTGCA |
| | | CTTTACAATCTGGACGAGAAGTCGACTCTGCGCCGCTCCC |
| | | ATGAAAACCCGTCTATCCGTGAGTTATACGACACTTATTTG |
| | | GGTGAGCCCTTAGGGCACAAAGCACACGAACTTTTACATAC |
| | | TCACTATGTAGCTGGCGGGGTCGAGGAGAAGGATGAGAAA |
| | | AAGTGACCAGGCATCAAATAAAACGAAAGGCTCAGTCGAAA |
| | | GACTGGGCCTTTCGTTTTATCTGTTGTTTGTCGGTGAACGC |
| | | TCTCTACTAGAGTCACACTGGCTCACCTTCGGGTGGGCCTT |
| | | TCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAG |
| | | TGCAATGCAGGCCGTCTTTTGGCGGGCCAGCGCCCCTGC |
| | | GGCGTCCGCGCGCGTCTGAACCGCCGTGTATGTGTTCCAG |
| | | TGACCGCACACGGCAAAGCATCTGCCACGCGCGAATACGC |
| | | TGGTGACTTTCTTCCAGGGACCACTATTAGTCATGCGTGGT |
| | | CCGTGGAGCGTGAAACTCATCACCGTTATCGTAATCCGGC |
| | | CGAGTGGATTAACGAGGCTGCCATCCACAAGGCGCTTGAA |
| | | ACGTCAAAGGCTGATGCTCAGGACGCAGGACGCGTGCGT |
| | | GAGATTTTGGCGAAGGCTAAGGAAAAGGCTTTTGTTACTGA |
| | | ACATGCGCCTGTAAATGCGGAAAGTAAATCCGAATTTGTAC |
| | | AAGGGCTTACCTTGGAGGAGTGCGCCACCTTAATTAACGTT |
| | | GATTCTAATAATGTCGAGTTGATGAATGAAATCTTCGACACT |
| | | GCGCTTGCTATCAAAGAACGTATCTACGGGAACCGCGTCG |
| | | TGCTGTTCGCGCCCTTGTACATTGCTAATCATTGTATGAAC |
| | | ACTTGCACTTATTGTGCGTTTCGCTCAGCCAATAAGGGCAT |
| | | GGAACGCTCTATTCTGACAGACGATGACCTGCGTGAAGAA |
| | | GTTGCCGCCTTGCAACGTCAAGGTCATCGCCGCATTTTAG |
| | | CCCTTACTGGAGAACATCCTAAATACACTTTCGACAATTTCT |
| | | TGCATGCAGTCAATGTAATCGCTTCCGTGAAGACAGAGCCT |
| | | GAAGGGTCCATTCGCCGTATCAATGTCGAAATCCCACCACT |
| | | TAGTGTCTCAGACATGCGCCGCCTTAAGAATACAGATTCAG |
| | | TTGGCACGTTCGTTCTGTTTCAAGAGACATATCACCGCGAC |
| | | ACATTCAAGGTAATGCATCCGTCAGGTCCAAAGAGCGACTT |
| | | TGATTTTCGCGTCTTGACCCAGGACCGCGCCATGCGCGCA |
| | | GGCTTGGACGATGTTGGTATTGGTGCTTTATTCGGGTTATA |
| | | TGATTACCGTTATGAAGTTTGCGCCATGTTAATGCATAGCG |
| | | AGCATTTGGAACGTGAGTACAACGCAGGACCACATACTATO |
| | | TCTGTTCCCCGTATGCGCCCCGCGGACGGAAGCGAGCTTA |
| | | GTATCGCGCCTCCTTATCCTGTTAATGATGCTGATTTTATGA |
| | | AATTAGTTGCGGTCCTTCGTATTGCTGTACCATACACAGGT |
| | | ATGATCCTTAGCACTCGTGAATCACCAGAAATGCGCTCGGC |
| | | GCTTCTGAAGTGTGGGATGAGTCAAATGAGTGCCGGCTCG |
| | | CGCACTGATGTAGGAGCCTATCACAAAGATCACACGTTAAG |
| | | TACAGAGGCGAATTTGAGTAAGTTAGCTGGGCAGTTTACCT |
| | | TGCAGGATGAACGTCCAACTAATGAGATTGTTAAGTGGCTG |
| | | ATGGAGGAGGGCTATGTCCCGTCTTGGTGCACAGCTTGTT |
| | | ACCGTCAAGGTCGTACGGGCGAAGACTTCATGAACATCTG |
| | | TAAGGCTGGTGACATTCACGATTTCTGTCATCCCAATAGTC |
| | | TGCTTACGCTTCAAGAGTACCTTATGGATTATGCAGATCCA |
| | | GACCTTCGTAAGAAAGGCGAGCAAGTAATTGCCCGTGAGA |
| | | TGGGACCGGACGCCTCTGAGCCGTTATCGGCGCAAAGCC |
| | | GTAAGCGCCTTGAACGCAAGATGAAACAAGTATTGGAGGG |
| | | GGAGCACGATGTATATCTTTAACCAGGCATCAAATAAAACG |
| | | AAAGGCTCAGTCGAAAGACTGGGCCTTTCGTTTTATCTGTT |
| | | GTTTGTCGGTGAACGCTCTCTACTAGAGTCACACTGGCTCA |
| | | CCTTCGGGTGGGCCTTTCTGCGTTTATA |
| 11 | pSB1C3 plasmid | TACTAGTAGCGGCCGCTGCAGTCCGGCAAAAAAGGGCAAG |
| | | GTGTCACCACCCTGCCCTTTTTCTTTAAAACCGAAAAGATTA |
| | | CTTCGCGTTATGCAGGCTTCCTCGCTCACTGACTCGCTGC |
| | | GCTCGGTCGTTCGGCTGCGGCGAGCGGTATCAGCTCACTC |
| | | AAAGGCGGTAATACGGTTATCCACAGAATCAGGGGATAAC |
| | | GCAGGAAAGAACATGTGAGCAAAAGGCCAGCAAAAGGCCA |
| | | GGAACCGTAAAAAGGCCGCGTTGCTGGCGTTTTTCCACAG |
| | | GCTCCGCCCCCCTGACGAGCATCACAAAAATCGACGCTCA |
| | | AGTCAGAGGTGGCGAAACCCGACAGGACTATAAAGATACC |
| | | AGGCGTTTCCCCCTGGAAGCTCCCTCGTGCGCTCTCCTGT |
| | | TCCGACCCTGCCGCTTACCGGATACCTGTCCGCCTTTCTC |
| | | CCTTCGGGAAGCGTGGCGCTTTCTCATAGCTCACGCTGTA |
| | | GGTATCTCAGTTCGGTGTAGGTCGTTCGCTCCAAGCTGGG |
| | | CTGTGTGCACGAACCCCCCGTTCAGCCCGACCGCTGCGCC |
| | | TTATCCGGTAACTATCGTCTTGAGTCCAACCCGGTAAGACA |
| | | CGACTTATCGCCACTGGCAGCAGCCACTGGTAACAGGATT |
| | | AGCAGAGCGAGGTATGTAGGCGGTGCTACAGAGTTCTTGA |
| | | AGTGGTGGCCTAACTACGGCTACACTAGAAGAACAGTATTT |
| | | GGTATCTGCGCTCTGCTGAAGCCAGTTACCTTCGGAAAAA |
| | | GAGTTGGTAGCTCTTGATCCGGCAAACAAACCACCGCTGG |
| | | TAGCGGTGGTTTTTTTGTTTGCAAGCAGCAGATTACGCGCA |
| | | GAAAAAAAGGATCTCAAGAAGATCCTTTGATCTTTTCTACG |
| | | GGGTCTGACGCTCAGTGGAACGAAAACTCACGTTAAGGGA |
| | | TTTTGGTCATGAGATTATCAAAAAGGATCTTCACCTAGATCC |
| | | TTTTAAATTAAAAATGAAGTTTTAAATCAATCTAAAGTATATA |
| | | TGAGTAAACTTGGTCTGACAGCTCGAGGCTTGGATTCTCAC |
| | | CAATAAAAAACGCCCGGCGGCAACCGAGCGTTCTGAACAA |
| | | ATCCAGATGGAGTTCTGAGGTCATTACTGGATCTATCAACA |
| | | GGAGTCCAAGCGAGCTCGATATCAAATTACGCCCCGCCCT |
| | | GCCACTCATCGCAGTACTGTTGTAATTCATTAAGCATTCTG |
| | | CCGACATGGAAGCCATCACAAACGGCATGATGAACCTGAA |
| | | TCGCCAGCGGCATCAGCACCTTGTCGCCTTGCGTATAATAT |
| | | TTGCCCATGGTGAAAACGGGGGCGAAGAAGTTGTCCATAT |
| | | TGGCCACGTTTAAATCAAAACTGGTGAAACTCACCCAGGGA |
| | | TTGGCTGAGACGAAAAACATATTCTCAATAAACCCTTTAGG |
| | | GAAATAGGCCAGGTTTTCACCGTAACACGCCACATCTTGCG |
| | | AATATATGTGTAGAAACTGCCGGAAATCGTCGTGGTATTCA |
| | | CTCCAGAGCGATGAAAACGTTTCAGTTTGCTCATGGAAAAC |
| | | GGTGTAACAAGGGTGAACACTATCCCATATCACCAGCTCAC |
| | | CGTCTTTCATTGCCATACGAAATTCCGGATGAGCATTCATC |
| | | AGGCGGGCAAGAATGTGAATAAAGGCCGGATAAAACTTGT |
| | | GCTTATTTTTCTTTACGGTCTTTAAAAAGGCCGTAATATCCA |
| | | GCTGAACGGTCTGGTTATAGGTACATTGAGCAACTGACTGA |
| | | AATGCCTCAAAATGTTCTTTACGATGCCATTGGGATATATCA |
| | | ACGGTGGTATATCCAGTGATTTTTTTCTCCATTTTAGCTTCC |
| | | TTAGCTCCTGAAAATCTCGATAACTCAAAAAATACGCCCGG |
| | | TAGTGATCTTATTTCATTATGGTGAAAGTTGGAACCTCTTAC |
| | | GTGCCCGATCAACTCGAGTGCCACCTGACGTCTAAGAAAC |
| | | CATTATTATCATGACATTAACCTATAAAAATAGGCGTATCAC |
| | | GAGGCAGAATTTCAGATAAAAAAAATCCTTAGCTTTCGCTA |
| | | AGGATGATTTCTGGAATTCGCGGCCGCTTCTAGAG |
| 12 | osmYp, 5′-3′ promoter | TTGTTAAATATAGATCACAATTTTGAAACCGCTCGGGATATC |
| | | AGCGAAAACATAAGCAAAAGTGAATGTTTTAAGAACATTCC |
| | | GTAA |
| 13 | gapAp, 5′-3′ | ATATTCCACCAGCTATTTGTTAGTGAATAAAAGGTTGCCTGT |
| | (gapA promoter) | AAAATTACAAAAACCTTACGCAGCGTCAAGCGGAATCGTGT |
| | | CAATCATTGCGACAAATCAATCCTGTGCCTAAGCATTACGC |
| | | GACTGACTCGCCTCACTCTTCCTTTGGGCTTGAGACCACAT |
| | | GGGGTCGGCGCCCCGAATTTTAAAGGGCAATTACGATAAA |
| | | AATGTGATTT |
| 14 | Nucleic acid sequence | AGCCTCTACCTCGCTGCGGTCGCCAGACCACTCTGTATGG |
| | encoding 6- | AACGAACCGTCTTTGTCGATGCGTTTGTATGTGTGGGCACC |
| | glucophosphonate | AAAAAAATCGCGCTGCCCTTGGATTAACGCTGCTGGAAGG |
| | dehydrogenase from | CGCTCGGCGCGTAATGAGTCGTAATAACTAAGACTACTAGC |
| | Corynebacterium | AAAGACGGGAATCGGCAACCCCAGTTGGGTGGCGGTCACA |
| | glutamicum, 5′-3′ | ATGACGCGGCGCCAGCTATCGATTAAGTCCCCAAGCTCGG |
| | (gnd gene sequence) | ATTTAAAGTAAGGATCTAACAGCAACGACTCTAACTCTGCG |
| | | TTCGCGTCATACGCTTCAACGATGCGATTTAAAAATTTAGC |
| | | GCGGATGATACAACCGCCGCGCCAAATAGTCGCTAAATCA |
| | | CGGGGGTCTACATCCCAGTTGTTCTCATCAGAACCGGCTTT |
| | | AATTTCGTCGAAACCTTGTGCGTAGGCTACCAACTTAGATG |
| | | CGTAAAGCGCACGACGGACATCCTCGACAAATTGAGCCTT |
| | | ATCTACGCCAAGCGCTTCAAGGTCTGTCAGTACCCCAGCG |
| | | GGAAGATTGCCCTGGGCGGCAGCACGTTGTGAAGTTGCAC |
| | | CGGACAGGGCACGGGCAAATACGGCCTCGCCAATTCCGG |
| | | TGGTTGCAATTCCCAAATCCAGAGCCGCTTTTACTGTCCAG |
| | | CGTCCTGTACCTTTTTGCCCCGCCGCATCCACGATAACATC |
| | | AATCAACGGCTTGCCTGTCTCTGCGTCGACCTGAGATAAAA |
| | | CCTCAGCCGTAATTTCGATCAAGTAGGAGTCAAGATCACCA |
| | | GCATTCCACTCCTTAAATACCTCGGCAATTTCGGCCGGCTG |
| | | CATTCCAGCCGCATAGCGCAGCAGGTGATACGCTTCCCCA |
| | | ATTACTTGCATATCGGCGTACTCAATGCCGTTATGTACCAT |
| | | CTTGACGAAGTGTCCTGCTCCGTCTGGCCCGATATGCGTA |
| | | ACACAAGGGGTACCATCCACGTTGGCAGCAATTGACTCAA |
| | | GCAAAGGGCCCAAGCTTTCATAACTCTTCGCAGGACCTCC |
| | | CGGCATAATAGAAGGTCCGTTCAAGGCCCCTTCTTCTCCG |
| | | CCGGAAATGCCCGCACCCACAAAGTGCAGTCCACGGGCAC |
| | | TGATCTCTTTTTCGCGGCGGATGGTATCCGTATACAGAGCG |
| | | TTGCCACCATCGATAATGATGTCGCCTTCGTCCATTGCGTC |
| | | TGCTAATTGATTGATGACGGCATCTGTAGCGTTGCCGGCCT |
| | | GGACCATGATGATGGCGCGACGGGGCTTCTCTAAACTCGC |
| | | TACAAACTCCTCGACCGTTGCGGAGGGAATAAAATTTCCCT |
| | | CGCTGCCATGATCCGCGATCAACTTATCCGTTTTGTCGGTA |
| | | CTGCGGTTATAGACTGCAACGGTGTTGCCGTTACGGGCAA |
| | | AATTGCGGGCAAGGTTTGAGCCCATAACCGCAAGCCCAAC |
| | | AACGCCAATTTGAGCCAGATTATCACCGTTTGTCAT |
| 15 | Nucleic acid sequence | ATGGCGAAGATTGCAATCAATGGCTTTGGGCGTATTGGCC |
| | encoding glyceraldehyde-3- | GCCTTGCGCTTCGTCGCATTCTGGAAGTACCGGGTCTGGA |
| | phosphate from Clostridium | AGTTGTGGCGATTAATGATCTGACCGATGCAAAGATGCTGG |
| | acetobutylicum, 5′-3′ | CACATTTGTTTAAATACGATTCTTCGCAAGGACGCTTCAAC |
| | (gapC gene sequence) | GGGGAGATTGAGGTGAAAGAAGGCGCGTTTGTCGTCAACG |
| | | GCAAAGAAGTCAAGGTATTCGCCGAGGCCGATCCAGAAAA |
| | | GTTGCCGTGGGGAGACCTGGGCATTGATGTGGTCCTTGAG |
| | | TGTACGGGCTTTTTCACTAAGAAAGAGAAGGCCGAGGCAC |
| | | ACGTCCGTGCAGGGGCGAAGAAGGTCGTGATCTCTGCTCC |
| | | AGCTGGGAATGATCTTAAAACCATTGTATTCAACGTCAACA |
| | | ATGAAGATCTGGACGGGACGGAAACGGTCATCTCGGGTGC |
| | | CAGCTGCACGACGAATTGTCTGGCTCCAATGGCAAAAGTC |
| | | TTAAATGACAAGTTTGGCATTGAGAAAGGATTCATGACAAC |
| | | TATTCATGCGTTTACCAACGACCAGAATACGTTAGATGGGC |
| | | CGCACCGTAAAGGAGACCTGCGTCGTGCACGTGCCGCGG |
| | | CCGTTTCCATCATCCCAAATTCAACAGGTGCCGCAAAAGCT |
| | | ATCTCGCAAGTTATCCCGGATCTGGCTGGTAAATTAGATGG |
| | | AAATGCACAACGTGTACCGGTGCCTACCGGTTCCATCACT |
| | | GAGCTGGTCTCAGTCCTGAAGAAAAAGGTAACGGTAGAAG |
| | | AGATTAATGCAGCGATGAAAGAAGCAGCCGATGAGTCTTTT |
| | | GGGTACACCGAAGATCCCATTGTATCCGCGGACGTCGTGG |
| | | GAATTAACTACGGTTCTTTGTTTGACGCAACTCTGACTAAAA |
| | | TTGTAGACGTGAATGGGTCTCAATTGGTTAAGACCGCTGCA |
| | | TGGTATGATAATGAGATGAGTTACACTTCTCAGCTGGTACG |
| | | CACACTTGCATATTTCGCCAAGATTGCTAAATAG |
| 16 | Nucleic acid sequence | GTCATACCAGGTCACTCCGTCACGTTCTGCCAATGCGATAG |
| | encoding glucose-6- | CGGTAGACGGTCCCCAGGTTCCCGATACGTAAGTCTTAGG |
| | phosphate dehydrogenase | TTTCATAGAGTTAGCTTTCCACCCCTCACGAATTCCGTCAAT |
| | from Zymomonas mobilis, | CCAGACCCACTGGGCCTCAACTTCGTCACGGCGTACAAAC |
| | 5′-3′ | AGAGTGGCGTCTCCCTCAATCAGGTCAAGCATCAGACGCT |
| | (zwf gene sequence) | CATAGGCGATACGGCGTTTGCGGTCCTTAAAAACATCAGTC |
| | | AACGACAAGTCCAGCCACACCTCGCGCATGTGGGCTCCGT |
| | | TACGGTCCAATCCGGGCTCTTTCACCATCATCGAGATTTGA |
| | | ATAGTCTCGTCCGGTTGTAAGACGATGCGTAACTTATTAGG |
| | | TTGCAAGATGCCGCCACTCGAACTGAAGATAGAGTGCGGT |
| | | ACAGGTTTGAACTGTACCACGATTTCGGAGCGACGGGCCG |
| | | GCAAGCGTTTACCCGTACGGATGTAAAATGGAACACCCTG |
| | | CCAACGCCAATTGTCCACATGGGCTTTAATTGCTACGAAAG |
| | | TTTCAGTATCACTTGGTTGCCCTAACTCGTCGATGTATCCT |
| | | GCAACTTCCTTTCCGCCAGAGACCCCCGCCCCGTACTGCC |
| | | CGGTTACGGTGTGGGTAAACACCGTATCATTGTTGATAGG |
| | | GCGCAAAGCGCGGAATACTTTAACCTTCTCATCACGCACAG |
| | | CATTGGCCTCCATGTGTGCGGGGGGCTCCATTGCGACCAA |
| | | CGCTACAAGCTGAAGAATATGACTCTGAACCATGTCACGCA |
| | | GGGAGCCACTACCGTCGAAGTAACCAATGCGCCCTTCAAG |
| | | TCCGACTGTTTCCGCGACCGAAATCTGTACGTGGTCAATCC |
| | | CCTTGCTATTCCAAAGGGGCTCGAACAATGCGTTCCCAAAA |
| | | CGCAAGGTCAAAAGATTCTGCACCGTTTCTTTGCCCAAATA |
| | | GTGGTCGATGCGGTAAACTTGCTTCTCTGAGAATACCTTCA |
| | | GTACAGCATCGTTAATATGGTCGCTGGAGGCAAGGTCTTGT |
| | | CCCAGGGGTTTTTCCAATGCCAAACGTGAGGTTGGCCCCG |
| | | CTAATCCCGCCTGCTTTAATCCAGCAATGGCGCCTTCAAAA |
| | | AGTGAAGGCGCCGTACTTAAGTAAATTGCGATGCCTTTTTC |
| | | TACGGGACCACACAGATCCGCTAATTTGCCGAACTGGGTG |
| | | GGGTCGGTGATGTCCACGGTAGCGTAAAAAAGCTTATTTAA |
| | | AAACTTCGCTTTAGCATCGTCATTCAAACGGTCAGAGGCCA |
| | | CGAAACGGTCCAGCGCTTTCTCCGCAAAGTCACGGAATCC |
| | | GTCAGTATCATACTCGGAACGCGAGGTGCATACGATACGC |
| | | AAGTCGTCCGCCAGTAAGCCGTCTGCATCAAGCCCATAAA |
| | | GGCTAGGCAGCAACATGCGTTGGCTTAAATCCCCGGTGCT |
| | | ACCGAACAGAATCATTGTTGACACAGTATTGGTCAT |
| 17 | Codon optimised nucleic | CCCGGGATGTCCGCATTAGTTCTGAAGCCATGTGCGGCGG |
| | acid sequence encoding | TCAGTATCCGCGGAAGCAGCTGCCGCGCCCGTCAAGTCGC |
| | HydA from Chlamydomonas | GCCGCGCGCGCCGTTAGCAGCGTCTACAGTTCGTGTTGCA |
| | reinhardtii, 5′-3′ (including | TTAGCAACACTTGAAGCTCCTGCGCGTCGCCTGGGCAACG |
| | restriction sites shown in | TCGCTTGTGCAGCAGCGGCTCCGGCGGCGGAGGCCCCTT |
| | bold) | TGAGCCACGTCCAACAAGCCTTAGCTGAATTGGCCAAACC |
| | | GAAAGATGACCCCACACGCAAGCATGTTTGTGTGCAAGTC |
| | | GCCCCAGCGGTCCGCGTCGCTATTGCCGAAACACTTGGAC |
| | | TTGCTCCCGGTGCCACGACCCCGAAGCAATTGGCGGAGG |
| | | GCTTACGCCGTCTGGGTTTCGACGAAGTATTCGACACGCTT |
| | | TTCGGGGCAGATTTGACAATTATGGAAGAAGGGTCAGAGTT |
| | | GTTGCATCGTTTAACGGAACATTTGGAGGCTCACCCTCACT |
| | | CTGACGAACCGTTGCCCATGTTTACCTCGTGTTGTCCGGGT |
| | | TGGATTGCGATGCTTGAGAAATCGTACCCGGATTTAATTCC |
| | | TTATGTGTCCTCGTGCAAATCTCCTCAAATGATGTTAGCCG |
| | | CGATGGTGAAGTCATATCTTGCCGAAAAGAAGGGCATCGC |
| | | ACCTAAAGACATGGTGATGGTGTCAATCATGCCTTGCACCC |
| | | GTAAGCAGTCCGAGGCCGATCGCGACTGGTTTTGTGTGGA |
| | | TGCTGACCCTACATTACGCCAGTTAGATCATGTTATTACAA |
| | | CCGTAGAGTTGGGTAACATCTTTAAGGAGCGTGGTATCAAT |
| | | TTGGCTGAGTTACCGGAAGGTGAATGGGACAACCCGATGG |
| | | GGGTGGGTTCGGGAGCCGGAGTATTATTTGGGACCACTGG |
| | | CGGCGTAATGGAGGCAGCATTACGTACGGCCTACGAACTG |
| | | TTCACAGGTACCCCGCTGCCTCGCCTGAGTTTGAGCGAGG |
| | | TGCGTGGTATGGACGGTATTAAGGAAACGAACATCACAATG |
| | | GTTCCTGCTCCCGGTTCAAAATTCGAGGAACTGTTGAAGCA |
| | | TCGCGCTGCCGCACGTGCAGAAGCAGCCGCACACGGTAC |
| | | TCCTGGTCCACTTGCCTGGGACGGCGGGGCCGGATTTACT |
| | | TCAGAAGACGGTCGTGGTGGAATTACTTTACGTGTCGCTGT |
| | | TGCGAATGGGCTGGGTAACGCTAAAAAGTTAATTACAAAAA |
| | | TGCAGGCCGGAGAGGCAAAATATGATTTCGTAGAGATTATG |
| | | GCCTGTCCCGCTGGTTGCGTAGGAGGTGGGGGCCAGCCC |
| | | CGTTCAACTGACAAAGCTATCACGCAGAAACGTCAAGCGG |
| | | CATTATACAATCTGGATGAAAAATCAACGCTTCGCCGTTCA |
| | | CATGAGAATCCATCAATCCGTGAATTGTACGATACTTATTTA |
| | | GGTGAGCCTCTGGGGCATAAGGCACACGAACTTCTGCATA |
| | | CTCATTACGTCGCGGGTGGAGTTGAGGAGAAGGATGAAAA |
| | | GAAGTAGGGATCC |
| 18 | Codon optimised nucleic | CCCGGGATGTACGTTTGCGTCGCACAAGTTACGTTACATGA |
| | acid sequence encoding | TTGCTTAGCATGCTCAGGGTGCATCACATCTGCGGAAACG |
| | HydA (Hyd1) from Volvox | GTTTTGCTTCAGCAACAGTCCGGGGATGAGTTCTTATCCCG |
| | carteri, 5′-3′ (including | CTTGGCAGACCCGCACACTACTGTCGTAGTTACCGTCTCG |
| | restriction sites shown in | CCCCAATCGCGCACAGCTTTGGCGGCTTACTATGGATTATG |
| | bold) | CCCGTCTCAGGCTCTTGCCCGCCTTGTGGGCTGGTTGAAG |
| | | TGGCTTGGGGTTCGCGCCGTATGGGATCTGACAACAGCCC |
| | | GCGATCTTGTATTGTTGGAAGAGGCAGCTGAGTTCATGAAC |
| | | CGTTGGGTCTGCATGTATGTTAGCGCCGGGCCTCTTCCGG |
| | | TCATGGGCAGTTCATGTCCTGGCACAGGCTGGGTTTGTTAT |
| | | GCTGAAAAGACGCACGGGACGCGTGTATTACCCTATTTGA |
| | | GCACCACCCGTTCACCCCAAGGAGCAATGGGTGGCTTGGT |
| | | AAAGTCCCTGGTGGCAGCAGCTTGGGGCGTTACTCCAGGG |
| | | TCTTTGTACCACGTCACAATTATGCCGTGTTATGACAAGAA |
| | | ATTGGAAGCATCTCGCGATGAATTAACCACAACGGCGACAA |
| | | CTACTACCGCTGCGGGGACAGACGCGGCTGGAGCAGGTG |
| | | CCGCCGTAGGCGGGCCTCTGCCTGAAGTGATGGTCCGTCA |
| | | TGCAACCGCAGCGCCTGATCCGTTGCTTCCGGGCGTGGTA |
| | | CCAGCCGACGACCAGCTGTACTCCCTTCCACATGGCTCCA |
| | | GCTCGGGTGGATACGCGGACTTCGTTTTCCGCACTGCCGC |
| | | GCGCGAGTTGTGGGGGGTAGAAGTTCCGCCAGGACCTTTA |
| | | CCATGGCGTACTCGTCGTAACGCCGACTTACAGCCTCGTG |
| | | TTGGTGCCCCAGGCCAATCTCTGGTCGTTGCGCGTGTGTA |
| | | TGGATTCCGCAATATTCAGACACTTTTACAGCAGCTTAAGC |
| | | GTGGACGCTGCCCTTATCATTATGTGGAAGTCATGGCTTGT |
| | | CCTAGCGGGTGTTTGAACGGAGGAGGACAAATCAAACCGG |
| | | GGCCAGGGGTAACGCCGCAACAATTAATTGAACAATTGGA |
| | | GCTTTTGTATGACGTTGCGGCACGTTCTCCAGCAGACAACC |
| | | CGGCGGTAGCAGCTTTATATGGCTCTTGGTTAGGCGGTCG |
| | | TCCAGGGGCACCCCCCGCTCGTCAACTGTTACATACGACA |
| | | TTTCGCGAGCGCGAAAAGACTGTAACTGCCGCAACCGTCA |
| | | CGAATTGGTAGGGATCC |
| 19 | Codon optimised nucleic | CCCGGGATGCCTCCAAAACCCCAACACGATGTAACAGGCG |
| | acid sequence encoding | TGGACTCCAATAACGCGATCATGATTGATTACGCAAAGTGC |
| | HydA (Hyd1) from Giardia | ATTGGCTGCAACATGTGCATCAAAGCATGCGACGTGCAAG |
| | lamblia 5′-3′ ((including | GTATTGGTGTTTACAAGCAGAACGAAAAACCGAAGTACCCG |
| | restriction sites shown in | CCTATCGTGAAGCTGAGCACCCTGTTTAATAGCGATTGTAT |
| | bold) | CGGCTGCGGTCAATGTGCAACGATTTGTCCGGTGGACGCG |
| | | ATTGCTCCAAAGAACAATCTGGAAATTTACAAAGGCGAATC |
| | | TGCTTCCAAAAAAGTTCGTGTGGCGTTGATCGCGCCAAGC |
| | | ACTCGCGTGGCGTTTGGAGATGTCTTCGGGCTGCCGATCG |
| | | GTACAAATACCATTTACTCCCTGATTCGCATGCTGAAGCAA |
| | | TATCTGGGTTTCGATTATGTGTTCGACGTGAATTTCGGCGC |
| | | AGATGAAACCACCGTCATCGACACCCAAGAGCTGTTGCAC |
| | | TTCAAACACGAGGGCCGTGGTCCGGTGTTCACCAGCTGCT |
| | | GCCCGGCGTGGGTTAACCTCTGTGAAATGAAGTACCCGGA |
| | | GCTGCTGCCCCAGGTTAGCACCGCTAAATCTTGTGTTGCAA |
| | | TGGTAGCGACCCTTGTAAAAAGACGTTGGGTTCAAGAACAT |
| | | TTAATCCCGAAGGGTATTGTAGACAGCGTTGACGATGTCTA |
| | | CGTGGCTGATATTATGCCGTGTACCGCGAAAAAGGATGAA |
| | | AGCATGCGTCCACAACTGAACCGCGATGTGGACATCTGCC |
| | | TGACGGTGCGTGAAGTTGCGGAGCACCTGTATTTTCTGCA |
| | | CGGCGCGCGCTTGACGCTGGAGGAAGTCGAGGCGGATGC |
| | | GTTGGTTTTGCGTCCGGGCCGTAGCACGCAGAAAAAGTGG |
| | | GACTTTGACGCTCCGTTTAACACCGTGTCTGGTGGCTCCCA |
| | | TATCTTTGGTAAGACCGGTGGCGTTGCCGAGACGTGTCTC |
| | | CGTTTCATCAGCTATATGAAAAAGTCGCCGATAGAAAACGT |
| | | TAAGGAGGAATTGCTGAAAGAGTTCAAGACGCCGGGTCAG |
| | | CTGGTGCAAACCGTTAAGCTGGTCAGCTGCGAGATCGCCG |
| | | GTGAGACTTATCGTGCCCTGATTGCGCATGGCGGTTCAGC |
| | | AATCAATGCCGCCGCGCGTATGGTGCTCAATAAAGAGGTG |
| | | GAGTGCGACGTTGTTGAGCAGATGGCCTGTCCGGGTGGCT |
| | | GCCAGAACGGTGGAGGCATGCCGAAGATCAAGGGTAAAAA |
| | | AGAGGCAGTTTTGACGCGTGCGTCTACCTTGGACATCCTG |
| | | GACGGTAAAGAGCGCTTTGCGAGCGCGGGCGAGAACAAA |
| | | ACTCTGTGGGGTTTCAACGGCTGCTTAACCGAACACGAAG |
| | | CGCACGAGTTGCTTCACACCCATTATCAGCATCGCCCGGT |
| | | GGAATCGCTGCTGCCGCAGTAGGGATCC |
| 20 | Codon optimised nucleic | CCCGGGATGCCTCCAAAACCCTCACACACTGTAACAGGCC |
| | acid sequence encoding | ACGACCACAACCACAGCATCCAGTTTGACTGGAGCAAGTG |
| | HydA (Hyd1) from | TATGGGCTGCGGTATGTGTGCAACCAAATGCACCTTCGGC |
| | Entamoeba nuttalli, 5′-3′ | GTATTGGTTAAGCAGCCGCCGAAGATCCCGCCATTTGTCC |
| | (including restriction sites | AGCCGAACCGTGAAAAACTGAGCCAAGAGAACACCGATAA |
| | shown in bold) | AACGCGTGTCCTGATCGATGAAAGCGAGTGCACCGGCTGC |
| | | GGTCAATGCTCTCTGGTGTGCAACTTTGGATCTATCACCCC |
| | | GATCGACCACCTCGTTGACACCTTCAAAGCAAAGGAGGCG |
| | | GGCAAAAAACTCGTGGCCATGATTGCCCCGAGCACTCGCC |
| | | TTGGTGTCGCTGAGGCTATGGGTATGCCGATTGGCTCCAC |
| | | CGCTATGGCGCAGCTGGTGCATTGTCTGCGTCTGATTGGT |
| | | TTTGACTACGTTTTTGACGTGGACGCGGGTGCGGATAAGA |
| | | CGACCATGGACGATTACGCGGAAGTTATCGAAATGAAAAA |
| | | GGAGGGTAAGGGTCCGGCGATCACCAGCTGCTGCCCGGC |
| | | GTGGATTGAGCTGGTGGAGAAAGAATATCCGGATTTGATC |
| | | CCGAACGTGTCCACTGCGCGTAGCCCGATCGGCTGCTTGG |
| | | CCGGTTGCATCAAACGTGGTTGGGCAAAAGATGTTGGCAT |
| | | CGCGGTAGAGGATTTGTACACCGTGGGTATCATGCCTTGT |
| | | ATTGCGAAGAAGACGGAATCCCAACGTCAGCAAATTCATCA |
| | | AGATTACGACGCAAGCTGTACGTCTAACGAGATTGCGGCG |
| | | TACTTCAAGAAGCACCTGCCACCGGAAGAGTGCAAATTCAC |
| | | CCAGGAGCGCGAGGAAGCTCTGGCAAAGACTGAGGACGG |
| | | CCAATGTGATCTGCCGTTCCGTCGCATTAGCGGTGGCTCC |
| | | AACATTTTTGGTAAGACCGGCGGGGTGTGTGAAACCGTGT |
| | | TGCGGGTGATTGCGCGTAATGCGGGTGTTGACTGGAATAC |
| | | GTGCACCGTTAACAAAGAGGAAACATTCAAACACGCCGCTA |
| | | GCGGTTCGACCATGACCAATCTGAGTGTTGACATCGGCGG |
| | | AACTATAATCACCGGGGCAGTTTGCCATGGTGGTTATGCAA |
| | | TCCGTCATGCCTGCGAACTGATTCGCAAAGGTGAACTGAAA |
| | | GTCGACGTTGTTGAGATGATGGCCTGTGTTGGCGGCTGCC |
| | | TGGGCGGTGCTGGCCAGCCTAAGATCCCGCCGGCGAAGA |
| | | AGCTGGAAATGGATAAGCGCAGAGTGATGTTAGATATTCTG |
| | | GACCAGCAAACGGACATTCGTGCAGCGAATGAAAACACCG |
| | | ACGTGCTAGGCTGGATTGATAAACACTTCGACCACCAGGG |
| | | TGCGCATCAACATCTGCACACCTATTTCACCCCGCGTTATC |
| | | AGAATTAGGGATCC |
| 21 | Codon optimised nucleic | CCCGGGATGAAGAACAAAACAGTATCAAATGATAAAATAGA |
| | acid sequence encoding | CAATAAACTGAACTGCTCCGAAAACCTGGAAAACCGTACCA |
| | HydA (Hyd1) from | TCGACAAAAGCTTCTACACCTTTTCACGTGATACCAGCAAG |
| | Ilyobacter, polytrophus 5′-3′ | TGCATTAAGTGCTACAAGTGCGTGAAAGTTTGCAAGGACAC |
| | (including restriction sites | TCAGGGCATTTCCGTTTTTCAGGTTGAGGAGGACGGCACG |
| | shown in bold) | GTGGGCATCAAAGAGGAGAATATGGCGGCCACCTTGTGTA |
| | | TCAGCTGTGGTCAATGTATTAAAGTGTGCACCGCAGGTGC |
| | | GCTGAAGGAGAAATCCAACATCTCTCTGTTGAAGGAGCAGT |
| | | TAAATAACCCGAATAAGCACGTTGTCGCGCAGCTGTCTCCA |
| | | AGCTTCAAACACACCATTGGTGATGGCTTCGGCATCAGCTC |
| | | GGGTACCGATACTTCCCCGAAAATCATCAGCGCTCTGAAA |
| | | GAAATTGGTTTCTCTAAGGTTTTCAGTACCGGTTTCGCCAG |
| | | CGACGTGAATATCGTTGAGACCAGCGCGGATCTGAAAAAA |
| | | CGCCTAGATGAAAACGGTCCGTTCCCGGTGTTCACCTCCA |
| | | CGTGCACGGGTTGGATTAACTATGCGGAAAAGTTTTGTCCG |
| | | GAGTTCCTGGGGCTGCTCTCTCCGTGCAAAAGCCCGCAAC |
| | | AGATCCTGGGTAGCCTGAGCAAGTCCTACTACGAAGAGAG |
| | | CATTGACATCAGCAGAGAAAATATCTTTAGCGTGGCGCTGA |
| | | TGCCTTGCATCGCCAAAAAGGATGAAGCTAATCGTTTTGAC |
| | | ATGAAGGACGAATATGGTAACAAAGATGTGGACCTGGTCCT |
| | | GACCGTGAATGAAGTTGCTAGCCTTTTGAACAAAAAGGGCA |
| | | TTGATTTAAACAATTACTCTAAGTTCGGTACTTTTGATAAGC |
| | | CGATGAAATCCGACACTGGTTCTTCGCGTATTAAGGCGGTT |
| | | ACGGGTGGCCTGGCAGAGGCTATCCTGCGCAACACCGCA |
| | | CACATGATCGGTGAAGATCCGTTTTCTGTAGACCTGAAGAA |
| | | GCTGCGTGGTATGGATGGTATTAAGCTCACGAGCGTGGTT |
| | | CTGGGCGGGAAAAAATTAAACATTGCGGTCGTGAACGGCA |
| | | TCAAGAACGTGCCGGTTATTCTGGACATGATTAAAGATGGC |
| | | ATTACCGAGTTCCACTTGGTCGAGGTTATGGCCTGTCCGG |
| | | GTGGCTGCGTCGGCGGCGGTGGTATCCCGTTGTCAGAAG |
| | | ACCCGGACATTATCCAAAAACGCGCAGAGAAAATCTACTCG |
| | | TACGACGCGAGCAGCGAAATTCGTTGTAGCTGGGAAAACC |
| | | CAGATGTTAAAACCCTGTATAGCGAGTACCTGAAGGAGCC |
| | | GCTGGGAGAGGAGTCTCAACGTTTGTTTCATTTTCATTATA |
| | | AAAACCGCCGTACCAAACGTATCTTCTAGGGATCC |
| 22 | Codon optimised nucleic | CCCGGGATGAGTGCTATGCTATCTTCATTAACAAATTCTCG |
| | acid sequence encoding | CTTGGGCGCTTACGTGCCGCAAGTGGCGGTAAGCCGTTAT |
| | HydA (Hyd1) from Volvox | GGTTCCAAGCCGGTCTACGTCCCGTGCGTGCCGTTTCGTA |
| | carteri, 5′-3′ (including | AGTGCACCTCGGCGGTGCCAGTGCCGGTTAAGACGGTGC |
| | restriction sites shown in | AAGAAGGTAATTCGGTCAGACTTATGCCAGCGGCGGCGGC |
| | bold) | TCCGGCGGGTGCGGCAGCGGACCCACATTGGAAACAAGC |
| | | ATTTGCAGAGCTTGATAAACCGAAAGCCGAACGTAAAGTTA |
| | | TGATCGCACAGGTTGCTCCGGCGGTGCGCGTTGCCATCTC |
| | | CGAGAGCTTTGGTCTGGCACCGGGGGCTACCACTCCGGG |
| | | TCAGCTGGCCGAGTCCCTGCGTTGTCTGGGTTTTGATATG |
| | | GTGTTCGACACCTTGTACGGCGCGGACCTGACCATTATGG |
| | | AAGAGGGCACTGAGTTGCTGTCCCGTCTGCAAGCGCAATT |
| | | GGAAGCCCACCCGCATAGCGAAGAACCGCTGCCGATGTTT |
| | | ACCAGCTGCTGTCCGGGCTGGATCGCCCTGTTGGAGAAGA |
| | | GCTATCCGGAATTAATTCCGTATGTGTCTAGCTGCAAGTCT |
| | | CCGCAGATGATGTTAGGCGCTATGGTGAAGACCTATCTGG |
| | | CAGAGAAGAAAGGCATCTCTCCGAGCGACATCTCCATGGT |
| | | TTCGATTATGCCGTGTGTGAGGAAGCAAGGTGAAGCGGAC |
| | | CGTGATTGGTTTTGCAGCGGCGGCGCCGGCGTTCGTGATG |
| | | TGGACCACGTTATTACCACGGCGGAGCTAGGCAACATCCT |
| | | GAAAGAACGTAATATCAACCTCCCCGAACTGCCGGAAGGC |
| | | GGTTGGGATGAGCCGCTGGGTTCGGGTAGCGGTGCGGGC |
| | | GTCGTTTTCGGTACAACCGGTGGTGTTATGGAAGCGGCGC |
| | | TGCGCACCGCGTACGAGTTGGTTACCCAGCAACCGCTGCC |
| | | TCGTTTGAATCTGAGTGAGGTTCGCGGTATGGACGGCATTA |
| | | AAGAAACCGAAATCAAGATGGTTCCGCCTCCGGGTAGCAA |
| | | ATTCGCCGAGCTTGTTGCCGCACGTGCAGCGGCGAAGGCT |
| | | ATGGATGAAGCGGCCGCGAGCGCAGGCGCGATTAAGTGG |
| | | GATGGTGGCAGCAACTTCACCGCGGACGACGGTGCTAAG |
| | | GGCATCACCCTGCGCGTGGCCGTGGCTAATGGTCTGGGAA |
| | | ACGCGAAAAAGCTGCTGACGAAAATGCAGACCGGTGAGTG |
| | | CAAATACGACTTCGTGGAAATTATGGCATGTCCGTCTGGGT |
| | | GCGTTGGTGGTGGTGGTCAGCCGCGCAGCACCGATAAACA |
| | | GGTGGCGGTCAAACGTCAGCAGGCACTGTACGACCTTGAC |
| | | GAGCGCGCAACGATCCGTCGTAGCCACGAGAACCCGGCT |
| | | GTCCAAAAAGCGTACGAAGATTTCCTGGGCGAGCCGAACA |
| | | GCCACAAAGCGCACGATCTGTTGCACACCCATTATGTTCCG |
| | | GGTGGCGTTGACACGGAGTAGGGATCC |
| 23 | Codon optimised nucleic | CCCGGGATGCTAGCGTCAAGTGCAACAGCTATGAAAGGCT |
| | acid sequence encoding | TCGCCAATAGCCTCCGTATGAAAGATTACAGCAGCACCGG |
| | HydA (Hyd1) from | TATTAACTTCGACATGACTAAGTGCATTAACTGCCAAAGCT |
| | Trichomonas vaginalis, 5′-3′ | GCGTTCGCGCCTGCACGAACATCGCTGGCCAAAACGTCTT |
| | (including restriction sites | GAAGAGCTTGACGGTGAACGGCAAGTCCGTTGTGCAGACC |
| | shown in bold) | GTTACCGGTAAACCGCTGGCGGAGACCAACTGCATCAGCT |
| | | GCGGTCAATGTACCCTTGGCTGCCCGAAGTTCACCATCTTC |
| | | GAGGCGGACGCCATCAATCCAGTTAAAGAGGTCTTGACCA |
| | | AGAAAAACGGTCGTATTGCCGTGTGTCAGATTGCTCCGGC |
| | | GATTCGTATCAACATGGCTGAAGCTCTGGGCGTTCCGGCT |
| | | GGTACGATCTCCTTGGGTAAGGTGGTGACCGCGCTGAAAC |
| | | GTCTGGGTTTTGACTACGTTTTCGATACCAATTTCGCTGCC |
| | | GATATGACCATCGTGGAGGAAGCGACCGAACTGGTTCAAC |
| | | GTTTGTCTGACAAAAACGCAGTGCTGCCTATGTTTACTTCA |
| | | TGCTGTCCAGCGTGGGTTAATTACGTAGAGAAATCCGATCC |
| | | GAGCTTGATCCCGCATCTGAGCTCTTGTCGTAGCCCGATG |
| | | AGCATGCTGAGCAGCGTTATCAAGAACGTCTTTCCGAAAAA |
| | | GATTGGTACGACCGCAGACAAAATCTACAATGTGGCGATCA |
| | | TGCCGTGCACCGCGAAGAAGGACGAAATTCAGCGCAGCCA |
| | | ATTTACCATGAAGGATGGTAAACAAGAGACAGGTGCGGTG |
| | | CTGACTTCTCGTGAACTCGCGAAAATGATTAAGGAGGCGAA |
| | | GATCAACTTCAAAGAGCTGCCGGATACGCCGTGTGATAACT |
| | | TTTATTCTGAAGCATCGGGCGGCGGTGCGATCTTTTGTGCA |
| | | ACCGGCGGCGTGATGGAAGCCGCGGTGCGCAGCGCGTAT |
| | | AAGTTCCTGACGAAGAAAGAGCTGGCACCGATTGACCTGC |
| | | AAGACGTTCGCGGTGTTGCGAGCGGCGTTAAACTGGCGGA |
| | | GGTGGATATTGCTGGTACCAAAGTAAAAGTGGCGGTTGCG |
| | | CACGGCATTAAGAACGCCATGACCCTGATTAAGAAGATCAA |
| | | ATCGGGTGAAGAGCAGTTTAAAGACGTTAAGTTCGTGGAA |
| | | GTGATGGCATGTCCGGGTGGCTGCGTGGTTGGTGGTGGC |
| | | AGCCCGAAGGCTAAGACCAAGAAAGCTGTCCAGGCACGAT |
| | | TGAATGCAACGTACTCCATTGACAAAAGCTCCAAACACAGA |
| | | ACTTCTCAGGACAACCCGCAGTTGTTACAGCTGTATAAAGA |
| | | ATCGTTTGAAGGTAAATTCGGTGGTCATGTCGCGCACCACC |
| | | TGCTGCACACCCATTATAAGAATCGTAAAGTAAATCCGTAG |
| | | GGATCC |
| 24 | Codon optimised nucleic | CCCGGGATGAAGGCTGTAGAGGGATTTGAATCAAAATATAA |
| | acid sequence encoding | ATTTTACGACAAGCGCGTGCCGATTGCGGACGACAACCCA |
| | HydA (Hyd1) from | GCTGTGCACTTTGACGAAACGAAATGCAAAAATTGTACCCT |
| | Megasphaera | TTGCCGTCGTGCGTGTGAAACTACGCAAACCGTGCTGGAC |
| | micronuciformis, 5′-3′ | TATTACAGCCTGGAACGCACCGGCGATGTTCCGGTTTGTG |
| | (including restriction sites | TTCACTGCGGTCAATGTGCAAATGCCTGCCCGTTTGGTGC |
| | shown in bold) | CATGATGGAAGTTGATGATACGAACTTAGTCAAAGCTGCGA |
| | | TCGCGGACCCGGATAAGGTGGTGGTTTTCCAAACGGCACC |
| | | GGCGGTACGTGTGGCCATTGCGGAAGAGTTCGGTGCGGA |
| | | GGCGGGTACATTCGCACAGGGTAAAATGATTTCAGCGTTG |
| | | AGAGCCCTGGGTGGCGACTACGTGTTCGACACCAACTTTG |
| | | GCGCTGACATGACCATCATGGAAGAAACTTCCGAGCTGGT |
| | | GCGTCGTATTACCACTGGTAACTTCGCAATGCCGCAGTTTA |
| | | CCAGCTGCTGCCCGGCGTGGGTTGAGTTTGCTGAGACCTT |
| | | CTACGCAGAATACATCCCGCATCTGTCCAGCGCGAAGAGC |
| | | CCGATTCTGATGCAAAACACCACGGAAAAAATCTGGTTTGC |
| | | CGAGAAGGCGGGCATCGATCCGAAGAAGATGGTGACGGTT |
| | | TGCGTTACCCCGTGTACCGCTAAAAAAGCCGAGATCAAGC |
| | | GCAAAGAATTGAATGCTGCGGCTGAGTACTGGCATATTGAT |
| | | GGCTTAAAGGACTCCGACATTTGCATCACGACCCGTGAACT |
| | | GGCACGTTGGCTGAAAGCGGAGAACATTGACTTCAATACC |
| | | CTGGATGATGGTATTTTTGATAGCCACCTGGGCGAGGCGA |
| | | GCGGTGGTGGCATCATCTTCGGCAGCACCGGTGGTGTCAT |
| | | GGAGTCCGCACTGCGCAGCGCGTATTACTTCTATACCGGT |
| | | AAACCGATGCCAGCGGAGTATATACCGTATGAACCGGTGC |
| | | GTGGCTTGGACGGCGTTAAAGAGGCGACCATCGACTTCAG |
| | | CGGTATCTCTCTGCATGTCGCAGTCGTGAGCGGTCTCGGC |
| | | AATGCGCGTCGTTTTCTGGATAAAATTATGGCGGACGGCAC |
| | | CTTCAAAGATTACACCTTTATCGAGTTCATGGCGTGCCAGG |
| | | GTGGCTGCATCAACGGTGGTGGCCAACCGAAGGTTAAGAT |
| | | GCCTCTGGTTCAGAAAACCAACCAGGCTCGCATGAATAGC |
| | | TTGTACAAACGTGATTCGGAGGTTTCGATTAAGGCCGCCTG |
| | | GGAAAACCCCGAAATCCAGGAACTCTACAGCGACTTCTAC |
| | | GGCCAGCCGCTGTCTGAGCGCTCTGAGAAGTATATTCACA |
| | | CCTTCTTTGAGGATAAGTCCGGCAACCTGGGTGAAGGTGG |
| | | TGCTGTGACCCCGCAAACGAACCCATTGTCTCCGAAGTATA |
| | | AGCCGATTGAATAGGGATCC |
| 25 | Codon optimised nucleic acid | CCCGGGATGTCAAAATATCAATTTTTAGATAGAAGGGTCCC |
| | sequence encoding | GATTGAGGACGGTAACATCGCTCTGGTTCAGGATTTGACTA |
| | HydA (Hyd1) from Veillonella | AGTGCAAAAATTGTAGCCTGTGTCGTAAAGCTTGCGCGGTC |
| | parvula, 5′-3′ (including | GATATGGGCGTCTTTGACTACTATGATCTGACCACGAATGG |
| | restriction sites shown in | TGACCACCCGATTTGCATCCACTGCGGTCAATGTGCGTCC |
| | bold) | ATCTGCCCATTCGATTCTATTAATGAACGCAGCGAGATCGA |
| | | TGAAGTTAAGGCGGCGATTGCGGACCCGAACAAAATCGTC |
| | | ATCTTCCAGACCGCACCGGCTGTGCGTGTGGGTCTTGGCG |
| | | AGGAGTTCGGCTTGGAGGCGGGTACCTTTGTGGAAGGCAA |
| | | GATGGTTGCAGCCCTGAGAAAGCTGGGCGGTGACTACATT |
| | | CTGGACACCAACTTTGGTGCGGACATGACCATCATGGAAG |
| | | AGGCGTCCGAGTTGCTGGAACGTGTTATCAACTCGGATGC |
| | | TGTTCTGCCGCAATTTACCAGCTGTTGTCCGGCGTGGGTTA |
| | | AGTTCGCGGAGACATTCTACCCGGAGTTCCTGCCTAACCT |
| | | GAGCACCGCGAAGTCCCCGATTGCCATGCAGGCACCGAC |
| | | CCAGAAAACCTATTTCGCCGAAAAAATGGGTCTGGACGCG |
| | | AAGCAGATTGTGGCGGTGGCCGTAACTCCGTGTACTGCCA |
| | | AGAAATTTGAGATTCGCCGTGACGAAATGAACAGCAGCGC |
| | | AGAGTATTGGAATACCCCAGAAATGCGTGATACCGATTACT |
| | | GCATCACCACGCGTGAGCTGGCAAAGTGGCTGCGCGCAG |
| | | AAGAAATCAACTTTGACGACCTCGAGGACAGCGCATTTGAT |
| | | CCGCTGATGGGTGAGGCTAGCGGTGGCGGTATTATTTTTG |
| | | GCAACACCGGCGGCGTTATGGAAGCTGCCATGCGCGCGG |
| | | CGTATAAAATGGCAACCGGTGAAGATGCCCCCCAAACCCT |
| | | TATCCCATTCGAGGCCATCAGAGGCATGGATGGTGCGCGC |
| | | GAAGCTGATGTTGTGATCGGTGACAAGACCCTGCACGTTG |
| | | CGGCGGTGCATGGTACGGGCAATTTACGTAAATTCATTGA |
| | | GCGCATGCGTGCGGAGAACATCCACTACGACTTCATCGAG |
| | | GTGATGGCATGCCGTGGTGGGTGCATCGGCGGCGGTGGC |
| | | CAACCGCGTGTTAAATTGCCGATGGCCGACAAAGCGCGCG |
| | | AAGCTCGTATCGCGTCTCTATACACCCGTGACGCAGAAGT |
| | | GACTGTAAAGGCCGCGTGCGATAATCCGGATATTCAGAAAT |
| | | TATATGCAGAGTTTTTCGACGGCAAACCGATGAGCCATAAA |
| | | GCACATCACATGCTGCATACCACGTTCGTGAATCGTAGCGA |
| | | AGATTTGGGTCCGAACGGTGCGTGCACCCCGGCGACGTG |
| | | CCCGACCAGTGTTCCGAACCTGAAAAAGGCTGCTGAAGCG |
| | | GCGAAGGCGGCGGCTGAAGTTAACTCTTAGGGATCC |
| 26 | Codon optimised nucleic | CCCGGGATGTCACAATTTGAATTTATAGATAAAAGGGTCCC |
| | acid sequence encoding | TATTGCGCTCGACAACCCGAGCATCTATCACGATATCTCCA |
| | HydA (Hyd1) from Veillonella | AATGCAAAAACTGCACCTTATGCCGTCGTGCGTGCGCCGA |
| | atypica, 5′-3′ (including | CGTGATGAGCGTTCTGGACTACTACGATCTGGAAGCGACC |
| | restriction sites shown in | GGTGACGTGCCAGTTTGTATTCATTGTGGACAATGTGCAGC |
| | bold) | GGCGTGCCCGTTTGACTCGATGCATGCAAAAAGCGAATTG |
| | | GATAAGGTGAAGGTGGCTATTGCCGATCCGGATAAAATCG |
| | | TCGTTATTCAGACCGCTCCGGCTGTACGCGTGTCGATTGG |
| | | CGAGGGTTTCGGCTTTGAACCGGGTACGTTTCTCGAGGGC |
| | | AAAATGGTTAGCGCGCTGCGTAAACTGGGTGCCGACTACG |
| | | TGGTCGACACGAACTTCGGCGCGGATCTGACCATTATGGA |
| | | GGAGGCGTCTGAATTGGTTGATCGTCTGAAGAACGGTGGC |
| | | ACGATCCCGCAATTCACCAGTTGCTGCCCGGCTTGGGTTC |
| | | GTTTTGCGGAAATCTACTTCCCGGAGTTGATCCCAAATCTG |
| | | TCTAGCACCCGTAGCTGCATCGCAATGGAGGCCGCTATGA |
| | | TTAAAACCTATTTCGCTGAGAAAAAGGGTATTAACCCGGCG |
| | | AATATCGTGTCCGTCTCCGTTAATCCGTGTACTGCGAAGAA |
| | | GGCTGAGACAAAGCGCGTGGAAGAAAATGCTGCCGCGCG |
| | | TTATTACGACGACGAATCCCTTGGCATGGATACCGACATCA |
| | | GCATTACCACCAGAGAATTTATCCGTTGGCTGAACGACGAA |
| | | GGCGTGGACTTCGAGTCCCTGGAGGACAGCCAGTTTGATG |
| | | ATCTTATCGGTATGGAAACTGGCGCGTCTATCATTTTTGGT |
| | | AATACCGGTGGCGTTATGGAGGCTGCTATGAGAACGGCGT |
| | | ACAAACTGATTACGGATAAGGAGCCGCCACCGTATGCACT |
| | | GACCCACCTGGAGGATGTGCGCGGTATGAACGGTGTGAAA |
| | | GAGGCGACGGTTCAGCTGGGTGATGACGTGACTCTGAGC |
| | | GTTGCGGTGGTTCACGGCGGTAAGAACACCCGTGATTTTTT |
| | | GAATGCGCTGAAGGAGAACGGCAAGCACTATGACTTCATC |
| | | GAAGTCATGGCATGTCCGGGTGGGTGCATTGGTGGCGGT |
| | | GGCCAACCGCGTACCAAACTGCCGCAGGCGGTTAAGACCA |
| | | AAGAGGCGCGTATCGGCGGTCTGTACAAGGCGGACGAAG |
| | | AATATAAGTACGTTGCCAGCTATGAATCACCGGAAATCCAA |
| | | GAGTTGTACAAGAACTTCTTGGGAGAACCTCTGGGTCACAA |
| | | AGCACATGAATTGCTGCATACCCACTTCACCGATCGCAGC |
| | | GCACAGTTAGGCGACCGCAAAGATGTTGTCCCGGAGACCT |
| | | GTCCGACCAGCCCGAAATACAAGGGTTAGGGATCC |
| 27 | Codon optimised nucleic | CCCGGGATGAAGCACCTATTTACAGAAAAAGTAGTTCCGAT |
| | acid sequence encoding | CGAGCTGGATAACCCGTCCATTCAGATTGACTTTGATAAGT |
| | HydA (Hyd1) from | GCATCAAGTGCGGTCTGTGTAAACGCGTTTGTGAAAACGA |
| | Peptoclostridium | GATTGGTGTTAATGGTTATTTCGACCTGGAGAAGACCGGTG |
| | bifermentans, 5′-3′ | ACATCGCCATCTGCATCAACTGCGGCCAATGTGTTCAGGC |
| | (including restriction sites | ATGTCCAAAAAAGGCGATCACCCAGGTTATCGATGTGGATC |
| | shown in bold) | GCGTGAAGGAAGCGATCAACGATCCGGAAAAGATTGTTAT |
| | | CTTCAGCACAGCGCCAGCTGTGCGTGTCGCACTGGGCGAA |
| | | GAATTTAACCTGGAAGAAGGCGCGTATGTTGAGGACAAAAT |
| | | GGTGGACGCCCTGCGTAAACTGGGTGGAGATTACGTTTTT |
| | | GACGTTACCTTTGGCGCCGATATGACCATCATGGAAGAGG |
| | | CGAACGAACTTGTTTCTCGTATCAAAAACGGCAAAGGCAAA |
| | | ACCCCGCAATTTACCAGCTGCTGCCCGTCCTGGGTTAAGTT |
| | | CGCGGAAACGTTCTATCCGGAGTTGATTCCGAATCTGTCTA |
| | | CCACGAAATCTCCGATTGGCATTCAGGGTGCTGTCATCAAG |
| | | ACCTATTTCGCACAGAAAGCAAATATCGACCCGGAGAAAAT |
| | | CGTGAACGTAACCATTACTCCGTGCACCGCTAAGAAGTAC |
| | | GAAATTGACCGTCCGGAGATGAATGCGAGCGCAAAATACA |
| | | ACAAAAGCGAAAATATGAGAGATAATGACATCATCCTCACC |
| | | ACTAAGGAGCTGGCGCAGTGGCTCCGTGACGAAGAGATCG |
| | | ACTTCAACGCTTTGGAGGGCTCGAAATTTGACAACATCTTG |
| | | GGCTTGGGTAGCGGTGCGGGCATTATCTTCGGTAATAGCG |
| | | GTGGTGTGATGGAAGCCGCGGTGCGCACGGTCTACAATAT |
| | | CCTCACCCATGAGAACCCACATAAAGAACTGCTGCACTTTA |
| | | ATCCGGTTCGTGGTCTGGAAGACGTGAAGGAGGCTACCCT |
| | | TACCATTGGCGATACCACCCTGCGCCTGGCAGCGGTGCAA |
| | | GGCACGGCGAACGTGCGTACGTTGATCGAAAAGCTGAAAT |
| | | CCGGTGAGGTGGAGTACGACTTCATAGAGGTAATGACTTG |
| | | CAAAGGTGGTTGTATTGGTGGCGCTGGTCAACCGAAGATG |
| | | AAAGCGCGTATTAGCAATGAGATGCGTCTCAAGCGCATTGA |
| | | GGGTCTGTACGATAAGGACAAGCACATTGCGGTCAAATGC |
| | | AGCTATGAAAACCCGGATGTCATCAACGTGTACAAGGAGTT |
| | | CTTCAAACAACCGCTGAGCCATTTATCCCACGAGTTGCTGC |
| | | ACACCACCTTTGAAAGCAAGCACGATATGTTGGGTCTGAAG |
| | | GATGACAACAACGTTTCGGATATTGGCTAGGGATCC |
| 28 | nirB promoter | GGTAAATTTGATGTACATCAAATGGATCCCCTTGCTGAATC |
| | | GTTAAGGTAGGCGGTAATAGAAAAGAAATCGAGGCAAAAAT |
| | | G |
| 29 | nar promoter | CTCTTGATCGTTATCAAATCCCAATACTGTGTTAGAGAGTAT |
| | | AATGCCCTTA |
| 30 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | alternative codon optimised | TTGCTGCGCGTGTCGGTGCCAAACCGGCAGTGCGTGGGG |
| | nucleic acid sequence | CGCGTCCCGCGTCGCGTATGTCTTGCATGGCCTATAAGGT |
| | encoding HydA from | TACACTGAAAACGCCATCAGGCGATAAAACAATCGAATGTC |
| | Chlamydomonas reinhardtii, | CGGCGGACACCTATATCCTGGATGCTGCGGAGGAAGCCG |
| | 5′-3′ (including restriction | GGCTGGACCTTCCCTATAGTTGCCGTGCGGGCGCCTGTTC |
| | sites shown in bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGTCCGC |
| | | ATTAGTTCTGAAGCCATGTGCGGCGGTCAGTATCCGCGGA |
| | | AGCAGCTGCCGCGCCCGTCAAGTCGCGCCGCGCGCGCCG |
| | | TTAGCAGCGTCTACAGTTCGTGTTGCATTAGCAACACTTGA |
| | | AGCTCCTGCGCGTCGCCTGGGCAACGTCGCTTGTGCAGCA |
| | | GCGGCTCCGGCGGCGGAGGCCCCTTTGAGCCACGTCCAA |
| | | CAAGCCTTAGCTGAATTGGCCAAACCGAAAGATGACCCCA |
| | | CACGCAAGCATGTTTGTGTGCAAGTCGCCCCAGCGGTCCG |
| | | CGTCGCTATTGCCGAAACACTTGGACTTGCTCCCGGTGCC |
| | | ACGACCCCGAAGCAATTGGCGGAGGGCTTACGCCGTCTG |
| | | GGTTTCGACGAAGTATTCGACACGCTTTTCGGGGCAGATTT |
| | | GACAATTATGGAAGAAGGGTCAGAGTTGTTGCATCGTTTAA |
| | | CGGAACATTTGGAGGCTCACCCTCACTCTGACGAACCGTT |
| | | GCCCATGTTTACCTCGTGTTGTCCGGGTTGGATTGCGATG |
| | | CTTGAGAAATCGTACCCGGATTTAATTCCTTATGTGTCCTC |
| | | GTGCAAATCTCCTCAAATGATGTTAGCCGCGATGGTGAAGT |
| | | CATATCTTGCCGAAAAGAAGGGCATCGCACCTAAAGACATG |
| | | GTGATGGTGTCAATCATGCCTTGCACCCGTAAGCAGTCCG |
| | | AGGCCGATCGCGACTGGTTTTGTGTGGATGCTGACCCTAC |
| | | ATTACGCCAGTTAGATCATGTTATTACAACCGTAGAGTTGG |
| | | GTAACATCTTTAAGGAGCGTGGTATCAATTTGGCTGAGTTA |
| | | CCGGAAGGTGAATGGGACAACCCGATGGGGGTGGGTTCG |
| | | GGAGCCGGAGTATTATTTGGGACCACTGGCGGCGTAATGG |
| | | AGGCAGCATTACGTACGGCCTACGAACTGTTCACAGGTAC |
| | | CCCGCTGCCTCGCCTGAGTTTGAGCGAGGTGCGTGGTATG |
| | | GACGGTATTAAGGAAACGAACATCACAATGGTTCCTGCTCC |
| | | CGGTTCAAAATTCGAGGAACTGTTGAAGCATCGCGCTGCC |
| | | GCACGTGCAGAAGCAGCCGCACACGGTACTCCTGGTCCAC |
| | | TTGCCTGGGACGGCGGGGCCGGATTTACTTCAGAAGACGG |
| | | TCGTGGTGGAATTACTTTACGTGTCGCTGTTGCGAATGGGC |
| | | TGGGTAACGCTAAAAAGTTAATTACAAAAATGCAGGCCGGA |
| | | GAGGCAAAATATGATTTCGTAGAGATTATGGCCTGTCCCGC |
| | | TGGTTGCGTAGGAGGTGGGGGCCAGCCCCGTTCAACTGA |
| | | CAAAGCTATCACGCAGAAACGTCAAGCGGCATTATACAATC |
| | | TGGATGAAAAATCAACGCTTCGCCGTTCACATGAGAATCCA |
| | | TCAATCCGTGAATTGTACGATACTTATTTAGGTGAGCCTCT |
| | | GGGGCATAAGGCACACGAACTTCTGCATACTCATTACGTC |
| | | GCGGGTGGAGTTGAGGAGAAGGATGAAAAGAAGTAGGGA |
| | | TCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 31 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Volvox carteri, | TACACTGAAAACGCCATCAGGCGATAAAACAATCGAATGTC |
| | 5′-3′ (including restriction | CGGCGGACACCTATATCCTGGATGCTGCGGAGGAAGCCG |
| | sites shown in bold) | GGCTGGACCTTCCCTATAGTTGCCGTGCGGGCGCCTGTTC |
| | | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGTACGT |
| | | TTGCGTCGCACAAGTTACGTTACATGATTGCTTAGCATGCT |
| | | CAGGGTGCATCACATCTGCGGAAACGGTTTTGCTTCAGCA |
| | | ACAGTCCGGGGATGAGTTCTTATCCCGCTTGGCAGACCCG |
| | | CACACTACTGTCGTAGTTACCGTCTCGCCCCAATCGCGCA |
| | | CAGCTTTGGCGGCTTACTATGGATTATGCCCGTCTCAGGCT |
| | | CTTGCCCGCCTTGTGGGCTGGTTGAAGTGGCTTGGGGTTC |
| | | GCGCCGTATGGGATCTGACAACAGCCCGCGATCTTGTATT |
| | | GTTGGAAGAGGCAGCTGAGTTCATGAACCGTTGGGTCTGC |
| | | ATGTATGTTAGCGCCGGGCCTCTTCCGGTCATGGGCAGTT |
| | | CATGTCCTGGCACAGGCTGGGTTTGTTATGCTGAAAAGAC |
| | | GCACGGGACGCGTGTATTACCCTATTTGAGCACCACCCGT |
| | | TCACCCCAAGGAGCAATGGGTGGCTTGGTAAAGTCCCTGG |
| | | TGGCAGCAGCTTGGGGCGTTACTCCAGGGTCTTTGTACCA |
| | | CGTCACAATTATGCCGTGTTATGACAAGAAATTGGAAGCAT |
| | | CTCGCGATGAATTAACCACAACGGCGACAACTACTACCGCT |
| | | GCGGGGACAGACGCGGCTGGAGCAGGTGCCGCCGTAGG |
| | | CGGGCCTCTGCCTGAAGTGATGGTCCGTCATGCAACCGCA |
| | | GCGCCTGATCCGTTGCTTCCGGGCGTGGTACCAGCCGAC |
| | | GACCAGCTGTACTCCCTTCCACATGGCTCCAGCTCGGGTG |
| | | GATACGCGGACTTCGTTTTCCGCACTGCCGCGCGCGAGTT |
| | | GTGGGGGGTAGAAGTTCCGCCAGGACCTTTACCATGGCGT |
| | | ACTCGTCGTAACGCCGACTTACAGCCTCGTGTTGGTGCCC |
| | | CAGGCCAATCTCTGGTCGTTGCGCGTGTGTATGGATTCCG |
| | | CAATATTCAGACACTTTTACAGCAGCTTAAGCGTGGACGCT |
| | | GCCCTTATCATTATGTGGAAGTCATGGCTTGTCCTAGCGGG |
| | | TGTTTGAACGGAGGAGGACAAATCAAACCGGGGCCAGGG |
| | | GTAACGCCGCAACAATTAATTGAACAATTGGAGCTTTTGTA |
| | | TGACGTTGCGGCACGTTCTCCAGCAGACAACCCGGCGGTA |
| | | GCAGCTTTATATGGCTCTTGGTTAGGCGGTCGTCCAGGGG |
| | | CACCCCCCGCTCGTCAACTGTTACATACGACATTTCGCGAG |
| | | CGCGAAAAGACTGTAACTGCCGCAACCGTCACGAATTGGT |
| | | AGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 32 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Giardia lamblia | TACACTGAAAACGCCATCAGGCGATAAAACAATCGAATGTC |
| | 5′-3′ ((including restriction | CGGCGGACACCTATATCCTGGATGCTGCGGAGGAAGCCG |
| | sites shown in bold) | GGCTGGACCTTCCCTATAGTTGCCGTGCGGGCGCCTGTTC |
| | | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGCCTCC |
| | | AAAACCCCAACACGATGTAACAGGCGTGGACTCCAATAAC |
| | | GCGATCATGATTGATTACGCAAAGTGCATTGGCTGCAACAT |
| | | GTGCATCAAAGCATGCGACGTGCAAGGTATTGGTGTTTACA |
| | | AGCAGAACGAAAAACCGAAGTACCCGCCTATCGTGAAGCT |
| | | GAGCACCCTGTTTAATAGCGATTGTATCGGCTGCGGTCAAT |
| | | GTGCAACGATTTGTCCGGTGGACGCGATTGCTCCAAAGAA |
| | | CAATCTGGAAATTTACAAAGGCGAATCTGCTTCCAAAAAAG |
| | | TTCGTGTGGCGTTGATCGCGCCAAGCACTCGCGTGGCGTT |
| | | TGGAGATGTCTTCGGGCTGCCGATCGGTACAAATACCATTT |
| | | ACTCCCTGATTCGCATGCTGAAGCAATATCTGGGTTTCGAT |
| | | TATGTGTTCGACGTGAATTTCGGCGCAGATGAAACCACCGT |
| | | CATCGACACCCAAGAGCTGTTGCACTTCAAACACGAGGGC |
| | | CGTGGTCCGGTGTTCACCAGCTGCTGCCCGGCGTGGGTTA |
| | | ACCTCTGTGAAATGAAGTACCCGGAGCTGCTGCCCCAGGT |
| | | TAGCACCGCTAAATCTTGTGTTGCAATGGTAGCGACCCTTG |
| | | TAAAAAGACGTTGGGTTCAAGAACATTTAATCCCGAAGGGT |
| | | ATTGTAGACAGCGTTGACGATGTCTACGTGGCTGATATTAT |
| | | GCCGTGTACCGCGAAAAAGGATGAAAGCATGCGTCCACAA |
| | | CTGAACCGCGATGTGGACATCTGCCTGACGGTGCGTGAAG |
| | | TTGCGGAGCACCTGTATTTTCTGCACGGCGCGCGCTTGAC |
| | | GCTGGAGGAAGTCGAGGCGGATGCGTTGGTTTTGCGTCCG |
| | | GGCCGTAGCACGCAGAAAAAGTGGGACTTTGACGCTCCGT |
| | | TTAACACCGTGTCTGGTGGCTCCCATATCTTTGGTAAGACC |
| | | GGTGGCGTTGCCGAGACGTGTCTCCGTTTCATCAGCTATAT |
| | | GAAAAAGTCGCCGATAGAAAACGTTAAGGAGGAATTGCTG |
| | | AAAGAGTTCAAGACGCCGGGTCAGCTGGTGCAAACCGTTA |
| | | AGCTGGTCAGCTGCGAGATCGCCGGTGAGACTTATCGTGC |
| | | CCTGATTGCGCATGGCGGTTCAGCAATCAATGCCGCCGCG |
| | | CGTATGGTGCTCAATAAAGAGGTGGAGTGCGACGTTGTTG |
| | | AGCAGATGGCCTGTCCGGGTGGCTGCCAGAACGGTGGAG |
| | | GCATGCCGAAGATCAAGGGTAAAAAAGAGGCAGTTTTGAC |
| | | GCGTGCGTCTACCTTGGACATCCTGGACGGTAAAGAGCGC |
| | | TTTGCGAGCGCGGGCGAGAACAAAACTCTGTGGGGTTTCA |
| | | ACGGCTGCTTAACCGAACACGAAGCGCACGAGTTGCTTCA |
| | | CACCCATTATCAGCATCGCCCGGTGGAATCGCTGCTGCCG |
| | | CAGTAGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 33 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Entamoeba | TACACTGAAAACGCCATCAGGCGATAAAACAATCGAATGTC |
| | nuttalli, 5′-3′ (including | CGGCGGACACCTATATCCTGGATGCTGCGGAGGAAGCCG |
| | restriction sites shown in | GGCTGGACCTTCCCTATAGTTGCCGTGCGGGCGCCTGTTC |
| | bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGCCTCC |
| | | AAAACCCTCACACACTGTAACAGGCCACGACCACAACCAC |
| | | AGCATCCAGTTTGACTGGAGCAAGTGTATGGGCTGCGGTA |
| | | TGTGTGCAACCAAATGCACCTTCGGCGTATTGGTTAAGCAG |
| | | CCGCCGAAGATCCCGCCATTTGTCCAGCCGAACCGTGAAA |
| | | AACTGAGCCAAGAGAACACCGATAAAACGCGTGTCCTGAT |
| | | CGATGAAAGCGAGTGCACCGGCTGCGGTCAATGCTCTCTG |
| | | GTGTGCAACTTTGGATCTATCACCCCGATCGACCACCTCGT |
| | | TGACACCTTCAAAGCAAAGGAGGCGGGCAAAAAACTCGTG |
| | | GCCATGATTGCCCCGAGCACTCGCCTTGGTGTCGCTGAGG |
| | | CTATGGGTATGCCGATTGGCTCCACCGCTATGGCGCAGCT |
| | | GGTGCATTGTCTGCGTCTGATTGGTTTTGACTACGTTTTTG |
| | | ACGTGGACGCGGGTGCGGATAAGACGACCATGGACGATTA |
| | | CGCGGAAGTTATCGAAATGAAAAAGGAGGGTAAGGGTCCG |
| | | GCGATCACCAGCTGCTGCCCGGCGTGGATTGAGCTGGTG |
| | | GAGAAAGAATATCCGGATTTGATCCCGAACGTGTCCACTGC |
| | | GCGTAGCCCGATCGGCTGCTTGGCCGGTTGCATCAAACGT |
| | | GGTTGGGCAAAAGATGTTGGCATCGCGGTAGAGGATTTGT |
| | | ACACCGTGGGTATCATGCCTTGTATTGCGAAGAAGACGGA |
| | | ATCCCAACGTCAGCAAATTCATCAAGATTACGACGCAAGCT |
| | | GTACGTCTAACGAGATTGCGGCGTACTTCAAGAAGCACCT |
| | | GCCACCGGAAGAGTGCAAATTCACCCAGGAGCGCGAGGA |
| | | AGCTCTGGCAAAGACTGAGGACGGCCAATGTGATCTGCCG |
| | | TTCCGTCGCATTAGCGGTGGCTCCAACATTTTTGGTAAGAC |
| | | CGGCGGGGTGTGTGAAACCGTGTTGCGGGTGATTGCGCG |
| | | TAATGCGGGTGTTGACTGGAATACGTGCACCGTTAACAAAG |
| | | AGGAAACATTCAAACACGCCGCTAGCGGTTCGACCATGAC |
| | | CAATCTGAGTGTTGACATCGGCGGAACTATAATCACCGGG |
| | | GCAGTTTGCCATGGTGGTTATGCAATCCGTCATGCCTGCG |
| | | AACTGATTCGCAAAGGTGAACTGAAAGTCGACGTTGTTGAG |
| | | ATGATGGCCTGTGTTGGCGGCTGCCTGGGCGGTGCTGGC |
| | | CAGCCTAAGATCCCGCCGGCGAAGAAGCTGGAAATGGATA |
| | | AGCGCAGAGTGATGTTAGATATTCTGGACCAGCAAACGGA |
| | | CATTCGTGCAGCGAATGAAAACACCGACGTGCTAGGCTGG |
| | | ATTGATAAACACTTCGACCACCAGGGTGCGCATCAACATCT |
| | | GCACACCTATTTCACCCCGCGTTATCAGAATTAGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 34 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from | TACACTGAAAACGCCATCAGGCGATAAAACAATCGAATGTC |
| | Ilyobacter, polytrophus 5′-3′ | CGGCGGACACCTATATCCTGGATGCTGCGGAGGAAGCCG |
| | (including restriction sites | GGCTGGACCTTCCCTATAGTTGCCGTGCGGGCGCCTGTTC |
| | shown in bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGAAGAACAAAAC |
| | | AGTATCAAATGATAAAATAGACAATAAACTGAACTGCTCCG |
| | | AAAACCTGGAAAACCGTACCATCGACAAAAGCTTCTACACC |
| | | TTTTCACGTGATACCAGCAAGTGCATTAAGTGCTACAAGTG |
| | | CGTGAAAGTTTGCAAGGACACTCAGGGCATTTCCGTTTTTC |
| | | AGGTTGAGGAGGACGGCACGGTGGGCATCAAAGAGGAGA |
| | | ATATGGCGGCCACCTTGTGTATCAGCTGTGGTCAATGTATT |
| | | AAAGTGTGCACCGCAGGTGCGCTGAAGGAGAAATCCAACA |
| | | TCTCTCTGTTGAAGGAGCAGTTAAATAACCCGAATAAGCAC |
| | | GTTGTCGCGCAGCTGTCTCCAAGCTTCAAACACACCATTGG |
| | | TGATGGCTTCGGCATCAGCTCGGGTACCGATACTTCCCCG |
| | | AAAATCATCAGCGCTCTGAAAGAAATTGGTTTCTCTAAGGT |
| | | TTTCAGTACCGGTTTCGCCAGCGACGTGAATATCGTTGAGA |
| | | CCAGCGCGGATCTGAAAAAACGCCTAGATGAAAACGGTCC |
| | | GTTCCCGGTGTTCACCTCCACGTGCACGGGTTGGATTAAC |
| | | TATGCGGAAAAGTTTTGTCCGGAGTTCCTGGGGCTGCTCT |
| | | CTCCGTGCAAAAGCCCGCAACAGATCCTGGGTAGCCTGAG |
| | | CAAGTCCTACTACGAAGAGAGCATTGACATCAGCAGAGAAA |
| | | ATATCTTTAGCGTGGCGCTGATGCCTTGCATCGCCAAAAAG |
| | | GATGAAGCTAATCGTTTTGACATGAAGGACGAATATGGTAA |
| | | CAAAGATGTGGACCTGGTCCTGACCGTGAATGAAGTTGCT |
| | | AGCCTTTTGAACAAAAAGGGCATTGATTTAAACAATTACTCT |
| | | AAGTTCGGTACTTTTGATAAGCCGATGAAATCCGACACTGG |
| | | TTCTTCGCGTATTAAGGCGGTTACGGGTGGCCTGGCAGAG |
| | | GCTATCCTGCGCAACACCGCACACATGATCGGTGAAGATC |
| | | CGTTTTCTGTAGACCTGAAGAAGCTGCGTGGTATGGATGGT |
| | | ATTAAGCTCACGAGCGTGGTTCTGGGCGGGAAAAAATTAAA |
| | | CATTGCGGTCGTGAACGGCATCAAGAACGTGCCGGTTATT |
| | | CTGGACATGATTAAAGATGGCATTACCGAGTTCCACTTGGT |
| | | CGAGGTTATGGCCTGTCCGGGTGGCTGCGTCGGCGGCGG |
| | | TGGTATCCCGTTGTCAGAAGACCCGGACATTATCCAAAAAC |
| | | GCGCAGAGAAAATCTACTCGTACGACGCGAGCAGCGAAAT |
| | | TCGTTGTAGCTGGGAAAACCCAGATGTTAAAACCCTGTATA |
| | | GCGAGTACCTGAAGGAGCCGCTGGGAGAGGAGTCTCAAC |
| | | GTTTGTTTCATTTTCATTATAAAAACCGCCGTACCAAACGTA |
| | | TCTTCTAGGGATCCGGGTCGAGGAGAAGGATGAGAAAAAG |
| | | TGACCAGGCATCAAATAAAACGAAAGGCTCAGTCGAAAGA |
| | | CTGGGCCTTTCGTTTTATCTGTTGTTTGTCGGTGAACGCTC |
| | | TCTACTAGAGTCACACTGGCTCACCTTCGGGTGGGCCTTTC |
| | | TGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 35 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Volvox carteri, | TACACTGAAAACGCCATCAGGCGATAAAACAATCGAATGTC |
| | 5′-3′ (including restriction | CGGCGGACACCTATATCCTGGATGCTGCGGAGGAAGCCG |
| | sites shown in bold) | GGCTGGACCTTCCCTATAGTTGCCGTGCGGGCGCCTGTTC |
| | | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGAGTGC |
| | | TATGCTATCTTCATTAACAAATTCTCGCTTGGGCGCTTACGT |
| | | GCCGCAAGTGGCGGTAAGCCGTTATGGTTCCAAGCCGGTC |
| | | TACGTCCCGTGCGTGCCGTTTCGTAAGTGCACCTCGGCGG |
| | | TGCCAGTGCCGGTTAAGACGGTGCAAGAAGGTAATTCGGT |
| | | CAGACTTATGCCAGCGGCGGCGGCTCCGGCGGGTGCGGC |
| | | AGCGGACCCACATTGGAAACAAGCATTTGCAGAGCTTGATA |
| | | AACCGAAAGCCGAACGTAAAGTTATGATCGCACAGGTTGCT |
| | | CCGGCGGTGCGCGTTGCCATCTCCGAGAGCTTTGGTCTGG |
| | | CACCGGGGGCTACCACTCCGGGTCAGCTGGCCGAGTCCC |
| | | TGCGTTGTCTGGGTTTTGATATGGTGTTCGACACCTTGTAC |
| | | GGCGCGGACCTGACCATTATGGAAGAGGGCACTGAGTTGC |
| | | TGTCCCGTCTGCAAGCGCAATTGGAAGCCCACCCGCATAG |
| | | CGAAGAACCGCTGCCGATGTTTACCAGCTGCTGTCCGGGC |
| | | TGGATCGCCCTGTTGGAGAAGAGCTATCCGGAATTAATTCC |
| | | GTATGTGTCTAGCTGCAAGTCTCCGCAGATGATGTTAGGC |
| | | GCTATGGTGAAGACCTATCTGGCAGAGAAGAAAGGCATCT |
| | | CTCCGAGCGACATCTCCATGGTTTCGATTATGCCGTGTGTG |
| | | AGGAAGCAAGGTGAAGCGGACCGTGATTGGTTTTGCAGCG |
| | | GCGGCGCCGGCGTTCGTGATGTGGACCACGTTATTACCAC |
| | | GGCGGAGCTAGGCAACATCCTGAAAGAACGTAATATCAAC |
| | | CTCCCCGAACTGCCGGAAGGCGGTTGGGATGAGCCGCTG |
| | | GGTTCGGGTAGCGGTGCGGGCGTCGTTTTCGGTACAACCG |
| | | GTGGTGTTATGGAAGCGGCGCTGCGCACCGCGTACGAGTT |
| | | GGTTACCCAGCAACCGCTGCCTCGTTTGAATCTGAGTGAG |
| | | GTTCGCGGTATGGACGGCATTAAAGAAACCGAAATCAAGAT |
| | | GGTTCCGCCTCCGGGTAGCAAATTCGCCGAGCTTGTTGCC |
| | | GCACGTGCAGCGGCGAAGGCTATGGATGAAGCGGCCGCG |
| | | AGCGCAGGCGCGATTAAGTGGGATGGTGGCAGCAACTTCA |
| | | CCGCGGACGACGGTGCTAAGGGCATCACCCTGCGCGTGG |
| | | CCGTGGCTAATGGTCTGGGAAACGCGAAAAAGCTGCTGAC |
| | | GAAAATGCAGACCGGTGAGTGCAAATACGACTTCGTGGAA |
| | | ATTATGGCATGTCCGTCTGGGTGCGTTGGTGGTGGTGGTC |
| | | AGCCGCGCAGCACCGATAAACAGGTGGCGGTCAAACGTCA |
| | | GCAGGCACTGTACGACCTTGACGAGCGCGCAACGATCCGT |
| | | CGTAGCCACGAGAACCCGGCTGTCCAAAAAGCGTACGAAG |
| | | ATTTCCTGGGCGAGCCGAACAGCCACAAAGCGCACGATCT |
| | | GTTGCACACCCATTATGTTCCGGGTGGCGTTGACACGGAG |
| | | TAGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 36 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Trichomonas | TACACTGAAAACGCCATCAGGCGATAAAACAATCGAATGTC |
| | vaginalis, 5′-3′ (including | CGGCGGACACCTATATCCTGGATGCTGCGGAGGAAGCCG |
| | restriction sites shown in | GGCTGGACCTTCCCTATAGTTGCCGTGCGGGCGCCTGTTC |
| | bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGCTAGC |
| | | GTCAAGTGCAACAGCTATGAAAGGCTTCGCCAATAGCCTC |
| | | CGTATGAAAGATTACAGCAGCACCGGTATTAACTTCGACAT |
| | | GACTAAGTGCATTAACTGCCAAAGCTGCGTTCGCGCCTGC |
| | | ACGAACATCGCTGGCCAAAACGTCTTGAAGAGCTTGACGG |
| | | TGAACGGCAAGTCCGTTGTGCAGACCGTTACCGGTAAACC |
| | | GCTGGCGGAGACCAACTGCATCAGCTGCGGTCAATGTACC |
| | | CTTGGCTGCCCGAAGTTCACCATCTTCGAGGCGGACGCCA |
| | | TCAATCCAGTTAAAGAGGTCTTGACCAAGAAAAACGGTCGT |
| | | ATTGCCGTGTGTCAGATTGCTCCGGCGATTCGTATCAACAT |
| | | GGCTGAAGCTCTGGGCGTTCCGGCTGGTACGATCTCCTTG |
| | | GGTAAGGTGGTGACCGCGCTGAAACGTCTGGGTTTTGACT |
| | | ACGTTTTCGATACCAATTTCGCTGCCGATATGACCATCGTG |
| | | GAGGAAGCGACCGAACTGGTTCAACGTTTGTCTGACAAAA |
| | | ACGCAGTGCTGCCTATGTTTACTTCATGCTGTCCAGCGTGG |
| | | GTTAATTACGTAGAGAAATCCGATCCGAGCTTGATCCCGCA |
| | | TCTGAGCTCTTGTCGTAGCCCGATGAGCATGCTGAGCAGC |
| | | GTTATCAAGAACGTCTTTCCGAAAAAGATTGGTACGACCGC |
| | | AGACAAAATCTACAATGTGGCGATCATGCCGTGCACCGCG |
| | | AAGAAGGACGAAATTCAGCGCAGCCAATTTACCATGAAGG |
| | | ATGGTAAACAAGAGACAGGTGCGGTGCTGACTTCTCGTGA |
| | | ACTCGCGAAAATGATTAAGGAGGCGAAGATCAACTTCAAAG |
| | | AGCTGCCGGATACGCCGTGTGATAACTTTTATTCTGAAGCA |
| | | TCGGGCGGCGGTGCGATCTTTTGTGCAACCGGCGGCGTG |
| | | ATGGAAGCCGCGGTGCGCAGCGCGTATAAGTTCCTGACGA |
| | | AGAAAGAGCTGGCACCGATTGACCTGCAAGACGTTCGCGG |
| | | TGTTGCGAGCGGCGTTAAACTGGCGGAGGTGGATATTGCT |
| | | GGTACCAAAGTAAAAGTGGCGGTTGCGCACGGCATTAAGA |
| | | ACGCCATGACCCTGATTAAGAAGATCAAATCGGGTGAAGA |
| | | GCAGTTTAAAGACGTTAAGTTCGTGGAAGTGATGGCATGTC |
| | | CGGGTGGCTGCGTGGTTGGTGGTGGCAGCCCGAAGGCTA |
| | | AGACCAAGAAAGCTGTCCAGGCACGATTGAATGCAACGTA |
| | | CTCCATTGACAAAAGCTCCAAACACAGAACTTCTCAGGACA |
| | | ACCCGCAGTTGTTACAGCTGTATAAAGAATCGTTTGAAGGT |
| | | AAATTCGGTGGTCATGTCGCGCACCACCTGCTGCACACCC |
| | | ATTATAAGAATCGTAAAGTAAATCCGTAGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 37 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Megasphaera | TACACTGAAAACGCCATCAGGCGATAAAACAATCGAATGTC |
| | micronuciformis, 5′-3′ | CGGCGGACACCTATATCCTGGATGCTGCGGAGGAAGCCG |
| | (including restriction sites | GGCTGGACCTTCCCTATAGTTGCCGTGCGGGCGCCTGTTC |
| | shown in bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGAAGGC |
| | | TGTAGAGGGATTTGAATCAAAATATAAATTTTACGACAAGC |
| | | GCGTGCCGATTGCGGACGACAACCCAGCTGTGCACTTTGA |
| | | CGAAACGAAATGCAAAAATTGTACCCTTTGCCGTCGTGCGT |
| | | GTGAAACTACGCAAACCGTGCTGGACTATTACAGCCTGGA |
| | | ACGCACCGGCGATGTTCCGGTTTGTGTTCACTGCGGTCAA |
| | | TGTGCAAATGCCTGCCCGTTTGGTGCCATGATGGAAGTTG |
| | | ATGATACGAACTTAGTCAAAGCTGCGATCGCGGACCCGGA |
| | | TAAGGTGGTGGTTTTCCAAACGGCACCGGCGGTACGTGTG |
| | | GCCATTGCGGAAGAGTTCGGTGCGGAGGCGGGTACATTC |
| | | GCACAGGGTAAAATGATTTCAGCGTTGAGAGCCCTGGGTG |
| | | GCGACTACGTGTTCGACACCAACTTTGGCGCTGACATGAC |
| | | CATCATGGAAGAAACTTCCGAGCTGGTGCGTCGTATTACCA |
| | | CTGGTAACTTCGCAATGCCGCAGTTTACCAGCTGCTGCCC |
| | | GGCGTGGGTTGAGTTTGCTGAGACCTTCTACGCAGAATAC |
| | | ATCCCGCATCTGTCCAGCGCGAAGAGCCCGATTCTGATGC |
| | | AAAACACCACGGAAAAAATCTGGTTTGCCGAGAAGGCGGG |
| | | CATCGATCCGAAGAAGATGGTGACGGTTTGCGTTACCCCG |
| | | TGTACCGCTAAAAAAGCCGAGATCAAGCGCAAAGAATTGAA |
| | | TGCTGCGGCTGAGTACTGGCATATTGATGGCTTAAAGGACT |
| | | CCGACATTTGCATCACGACCCGTGAACTGGCACGTTGGCT |
| | | GAAAGCGGAGAACATTGACTTCAATACCCTGGATGATGGTA |
| | | TTTTTGATAGCCACCTGGGCGAGGCGAGCGGTGGTGGCAT |
| | | CATCTTCGGCAGCACCGGTGGTGTCATGGAGTCCGCACTG |
| | | CGCAGCGCGTATTACTTCTATACCGGTAAACCGATGCCAG |
| | | CGGAGTATATACCGTATGAACCGGTGCGTGGCTTGGACGG |
| | | CGTTAAAGAGGCGACCATCGACTTCAGCGGTATCTCTCTG |
| | | CATGTCGCAGTCGTGAGCGGTCTCGGCAATGCGCGTCGTT |
| | | TTCTGGATAAAATTATGGCGGACGGCACCTTCAAAGATTAC |
| | | ACCTTTATCGAGTTCATGGCGTGCCAGGGTGGCTGCATCA |
| | | ACGGTGGTGGCCAACCGAAGGTTAAGATGCCTCTGGTTCA |
| | | GAAAACCAACCAGGCTCGCATGAATAGCTTGTACAAACGTG |
| | | ATTCGGAGGTTTCGATTAAGGCCGCCTGGGAAAACCCCGA |
| | | AATCCAGGAACTCTACAGCGACTTCTACGGCCAGCCGCTG |
| | | TCTGAGCGCTCTGAGAAGTATATTCACACCTTCTTTGAGGA |
| | | TAAGTCCGGCAACCTGGGTGAAGGTGGTGCTGTGACCCCG |
| | | CAAACGAACCCATTGTCTCCGAAGTATAAGCCGATTGAATA |
| | | GGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACO |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 38 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Veillonella | TACACTGAAAACGCCATCAGGCGATAAAACAATCGAATGTC |
| | parvula, 5′-3′ (including | CGGCGGACACCTATATCCTGGATGCTGCGGAGGAAGCCG |
| | restriction sites shown in | GGCTGGACCTTCCCTATAGTTGCCGTGCGGGCGCCTGTTC |
| | bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGTCAAA |
| | | ATATCAATTTTTAGATAGAAGGGTCCCGATTGAGGACGGTA |
| | | ACATCGCTCTGGTTCAGGATTTGACTAAGTGCAAAAATTGT |
| | | AGCCTGTGTCGTAAAGCTTGCGCGGTCGATATGGGCGTCT |
| | | TTGACTACTATGATCTGACCACGAATGGTGACCACCCGATT |
| | | TGCATCCACTGCGGTCAATGTGCGTCCATCTGCCCATTCGA |
| | | TTCTATTAATGAACGCAGCGAGATCGATGAAGTTAAGGCGG |
| | | CGATTGCGGACCCGAACAAAATCGTCATCTTCCAGACCGC |
| | | ACCGGCTGTGCGTGTGGGTCTTGGCGAGGAGTTCGGCTTG |
| | | GAGGCGGGTACCTTTGTGGAAGGCAAGATGGTTGCAGCCC |
| | | TGAGAAAGCTGGGCGGTGACTACATTCTGGACACCAACTTT |
| | | GGTGCGGACATGACCATCATGGAAGAGGCGTCCGAGTTGC |
| | | TGGAACGTGTTATCAACTCGGATGCTGTTCTGCCGCAATTT |
| | | ACCAGCTGTTGTCCGGCGTGGGTTAAGTTCGCGGAGACAT |
| | | TCTACCCGGAGTTCCTGCCTAACCTGAGCACCGCGAAGTC |
| | | CCCGATTGCCATGCAGGCACCGACCCAGAAAACCTATTTC |
| | | GCCGAAAAAATGGGTCTGGACGCGAAGCAGATTGTGGCGG |
| | | TGGCCGTAACTCCGTGTACTGCCAAGAAATTTGAGATTCGC |
| | | CGTGACGAAATGAACAGCAGCGCAGAGTATTGGAATACCC |
| | | CAGAAATGCGTGATACCGATTACTGCATCACCACGCGTGA |
| | | GCTGGCAAAGTGGCTGCGCGCAGAAGAAATCAACTTTGAC |
| | | GACCTCGAGGACAGCGCATTTGATCCGCTGATGGGTGAGG |
| | | CTAGCGGTGGCGGTATTATTTTTGGCAACACCGGCGGCGT |
| | | TATGGAAGCTGCCATGCGCGCGGCGTATAAAATGGCAACC |
| | | GGTGAAGATGCCCCCCAAACCCTTATCCCATTCGAGGCCA |
| | | TCAGAGGCATGGATGGTGCGCGCGAAGCTGATGTTGTGAT |
| | | CGGTGACAAGACCCTGCACGTTGCGGCGGTGCATGGTAC |
| | | GGGCAATTTACGTAAATTCATTGAGCGCATGCGTGCGGAG |
| | | AACATCCACTACGACTTCATCGAGGTGATGGCATGCCGTG |
| | | GTGGGTGCATCGGCGGCGGTGGCCAACCGCGTGTTAAATT |
| | | GCCGATGGCCGACAAAGCGCGCGAAGCTCGTATCGCGTCT |
| | | CTATACACCCGTGACGCAGAAGTGACTGTAAAGGCCGCGT |
| | | GCGATAATCCGGATATTCAGAAATTATATGCAGAGTTTTTC |
| | | GACGGCAAACCGATGAGCCATAAAGCACATCACATGCTGC |
| | | ATACCACGTTCGTGAATCGTAGCGAAGATTTGGGTCCGAAC |
| | | GGTGCGTGCACCCCGGCGACGTGCCCGACCAGTGTTCCG |
| | | AACCTGAAAAAGGCTGCTGAAGCGGCGAAGGCGGCGGCT |
| | | GAAGTTAACTCTTAGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 39 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Veillonella | TACACTGAAAACGCCATCAGGCGATAAAACAATCGAATGTC |
| | atypica, 5′-3′ (including | CGGCGGACACCTATATCCTGGATGCTGCGGAGGAAGCCG |
| | restriction sites shown in | GGCTGGACCTTCCCTATAGTTGCCGTGCGGGCGCCTGTTC |
| | bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGTCACA |
| | | ATTTGAATTTATAGATAAAAGGGTCCCTATTGCGCTCGACA |
| | | ACCCGAGCATCTATCACGATATCTCCAAATGCAAAAACTGC |
| | | ACCTTATGCCGTCGTGCGTGCGCCGACGTGATGAGCGTTC |
| | | TGGACTACTACGATCTGGAAGCGACCGGTGACGTGCCAGT |
| | | TTGTATTCATTGTGGACAATGTGCAGCGGCGTGCCCGTTTG |
| | | ACTCGATGCATGCAAAAAGCGAATTGGATAAGGTGAAGGT |
| | | GGCTATTGCCGATCCGGATAAAATCGTCGTTATTCAGACCG |
| | | CTCCGGCTGTACGCGTGTCGATTGGCGAGGGTTTCGGCTT |
| | | TGAACCGGGTACGTTTCTCGAGGGCAAAATGGTTAGCGCG |
| | | CTGCGTAAACTGGGTGCCGACTACGTGGTCGACACGAACT |
| | | TCGGCGCGGATCTGACCATTATGGAGGAGGCGTCTGAATT |
| | | GGTTGATCGTCTGAAGAACGGTGGCACGATCCCGCAATTC |
| | | ACCAGTTGCTGCCCGGCTTGGGTTCGTTTTGCGGAAATCTA |
| | | CTTCCCGGAGTTGATCCCAAATCTGTCTAGCACCCGTAGCT |
| | | GCATCGCAATGGAGGCCGCTATGATTAAAACCTATTTCGCT |
| | | GAGAAAAAGGGTATTAACCCGGCGAATATCGTGTCCGTCT |
| | | CCGTTAATCCGTGTACTGCGAAGAAGGCTGAGACAAAGCG |
| | | CGTGGAAGAAAATGCTGCCGCGCGTTATTACGACGACGAA |
| | | TCCCTTGGCATGGATACCGACATCAGCATTACCACCAGAGA |
| | | ATTTATCCGTTGGCTGAACGACGAAGGCGTGGACTTCGAG |
| | | TCCCTGGAGGACAGCCAGTTTGATGATCTTATCGGTATGGA |
| | | AACTGGCGCGTCTATCATTTTTGGTAATACCGGTGGCGTTA |
| | | TGGAGGCTGCTATGAGAACGGCGTACAAACTGATTACGGA |
| | | TAAGGAGCCGCCACCGTATGCACTGACCCACCTGGAGGAT |
| | | GTGCGCGGTATGAACGGTGTGAAAGAGGCGACGGTTCAG |
| | | CTGGGTGATGACGTGACTCTGAGCGTTGCGGTGGTTCACG |
| | | GCGGTAAGAACACCCGTGATTTTTTGAATGCGCTGAAGGA |
| | | GAACGGCAAGCACTATGACTTCATCGAAGTCATGGCATGTC |
| | | CGGGTGGGTGCATTGGTGGCGGTGGCCAACCGCGTACCA |
| | | AACTGCCGCAGGCGGTTAAGACCAAAGAGGCGCGTATCGG |
| | | CGGTCTGTACAAGGCGGACGAAGAATATAAGTACGTTGCC |
| | | AGCTATGAATCACCGGAAATCCAAGAGTTGTACAAGAACTT |
| | | CTTGGGAGAACCTCTGGGTCACAAAGCACATGAATTGCTG |
| | | CATACCCACTTCACCGATCGCAGCGCACAGTTAGGCGACC |
| | | GCAAAGATGTTGTCCCGGAGACCTGTCCGACCAGCCCGAA |
| | | ATACAAGGGTTAGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |
| 40 | Exemplary HPGC (hydrogen | CTAGAGGGCTTTACACTTTATGCTTCCGGCTCGTATGTTGT |
| | producing gene cluster) with | GTGGAAAGAAGGAGATATACATATGGCGATGCGTTCCACAT |
| | codon optimised nucleic acid | TTGCTGCGCGTGTCGGTGCCAAACCGGCAGTGCGTGGGG |
| | sequence encoding HydA | CGCGTCCCGCGTCGCGTATGTCTTGCATGGCCTATAAGGT |
| | (Hyd1) from Peptoclostridium | TACACTGAAAACGCCATCAGGCGATAAAACAATCGAATGTC |
| | bifermentans, 5′-3′ | CGGCGGACACCTATATCCTGGATGCTGCGGAGGAAGCCG |
| | (including restriction sites | GGCTGGACCTTCCCTATAGTTGCCGTGCGGGCGCCTGTTC |
| | shown in bold) | ATCCTGTGCTGGCAAAGTGGCAGCTGGAACGGTAGATCAA |
| | | TCTGATCAGTCCTTTCTGGATGACGCTCAGATGGGGAACG |
| | | GATTCGTCCTGACATGTGTCGCGTATCCTACAAGCGACTGC |
| | | ACTATCCAAACTCACCAAGAGGAGGCTTTATATTAAAAGAA |
| | | GGAGATATACATATGCAAACTGTTCGCGCTCCAGCAGCTTC |
| | | AGGTGTTGCCACACGTGTCGCAGGTCGTCGTATGTGTCGT |
| | | CCGGTTGCGGCGACGAAGGCTTCCACGGCTGTTACCACAG |
| | | ACATGTCGAAGCGCACTGTTCCAACCAAGTTAGAGGAAGG |
| | | TGAAATGCCGTTGAACACGTACAGTAATAAAGCTCCGTTCA |
| | | AGGCAAAGGTTCGTTCCGTGGAAAAAATCACAGGACCAAA |
| | | AGCCACAGGTGAGACGTGCCACATCATTATTGAAACCGAG |
| | | GGGAAGATCCCGTTTTGGGAGGGACAATCGTACGGTGTAA |
| | | TTCCGCCGGGGACCAAGATCAACTCTAAGGGAAAAGAAGT |
| | | GCCTCATGGTACTCGTCTTTATTCGATTGCTTCTAGTCGTTA |
| | | CGGAGATGACTTCGATGGTCAAACGGCATCGCTGTGTGTT |
| | | CGCCGCGCGGTATACGTCGATCCAGAGACTGGAAAGGAG |
| | | GACCCCGCGAAAAAAGGACTGTGTAGTAACTTTTTGTGTGA |
| | | TGCCACACCAGGCACGGAAATTTCCATGACAGGGCCCACA |
| | | GGAAAAGTATTGCTTCTGCCAGCAGACGCGAACGCGCCAT |
| | | TAATCTGTGTCGCAACGGGGACTGGAATCGCGCCTTTTCG |
| | | CTCATTCTGGCGCCGTTGCTTCATCGAGAATGTCCCAAGTT |
| | | ATAAGTTCACTGGCCTTTTCTGGTTGTTTATGGGTGTCGCT |
| | | AACTCTGATGCTAAATTGTACGATGAGGAACTGCAAGCTAT |
| | | CGCAAAAGCGTATCCGGGACAATTCCGCCTGGACTATGCC |
| | | TTGTCTCGTGAACAAAATAATCGCAAGGGCGGGAAAATGTA |
| | | CATCCAAGATAAGGTTGAAGAGTATGCCGACGAAATTTTCG |
| | | ATTTATTGGATAATGGGGCGCACATGTACTTCTGCGGGTTA |
| | | AAGGGGATGATGCCAGGCATCCAAGATATGTTAGAACGCG |
| | | TTGCAAAAGAAAAGGGGCTGAACTACGAAGAGTGGGTCGA |
| | | GGGGTTAAAGCACAAGAATCAATGGCATGTTGAAGTCTACT |
| | | AACCAGGCATCAAATAAAACGAAAGGCTCAGTCGAAAGACT |
| | | GGGCCTTTCGTTTTATCTGTTGTTTGTCGGTGAACGCTCTC |
| | | TACTAGAGTCACACTGGCTCACCTTCGGGTGGGCCTTTCT |
| | | GCGTTTATATA |
| | | CTAGAGGGCTTTACACTTTATGCTTCCGGCTCGTA |
| | | TGTTGTGTGGAAAGAAGGAGATATACAT |
| | | ATGTCCGCATTAGTACTTAAGCCCGGGATGAAGCA |
| | | CCTATTTACAGAAAAAGTAGTTCCGATCGAGCTGGATAACC |
| | | CGTCCATTCAGATTGACTTTGATAAGTGCATCAAGTGCGGT |
| | | CTGTGTAAACGCGTTTGTGAAAACGAGATTGGTGTTAATGG |
| | | TTATTTCGACCTGGAGAAGACCGGTGACATCGCCATCTGCA |
| | | TCAACTGCGGCCAATGTGTTCAGGCATGTCCAAAAAAGGC |
| | | GATCACCCAGGTTATCGATGTGGATCGCGTGAAGGAAGCG |
| | | ATCAACGATCCGGAAAAGATTGTTATCTTCAGCACAGCGCC |
| | | AGCTGTGCGTGTCGCACTGGGCGAAGAATTTAACCTGGAA |
| | | GAAGGCGCGTATGTTGAGGACAAAATGGTGGACGCCCTGC |
| | | GTAAACTGGGTGGAGATTACGTTTTTGACGTTACCTTTGGC |
| | | GCCGATATGACCATCATGGAAGAGGCGAACGAACTTGTTT |
| | | CTCGTATCAAAAACGGCAAAGGCAAAACCCCGCAATTTACC |
| | | AGCTGCTGCCCGTCCTGGGTTAAGTTCGCGGAAACGTTCT |
| | | ATCCGGAGTTGATTCCGAATCTGTCTACCACGAAATCTCCG |
| | | ATTGGCATTCAGGGTGCTGTCATCAAGACCTATTTCGCACA |
| | | GAAAGCAAATATCGACCCGGAGAAAATCGTGAACGTAACC |
| | | ATTACTCCGTGCACCGCTAAGAAGTACGAAATTGACCGTCC |
| | | GGAGATGAATGCGAGCGCAAAATACAACAAAAGCGAAAAT |
| | | ATGAGAGATAATGACATCATCCTCACCACTAAGGAGCTGGC |
| | | GCAGTGGCTCCGTGACGAAGAGATCGACTTCAACGCTTTG |
| | | GAGGGCTCGAAATTTGACAACATCTTGGGCTTGGGTAGCG |
| | | GTGCGGGCATTATCTTCGGTAATAGCGGTGGTGTGATGGA |
| | | AGCCGCGGTGCGCACGGTCTACAATATCCTCACCCATGAG |
| | | AACCCACATAAAGAACTGCTGCACTTTAATCCGGTTCGTGG |
| | | TCTGGAAGACGTGAAGGAGGCTACCCTTACCATTGGCGAT |
| | | ACCACCCTGCGCCTGGCAGCGGTGCAAGGCACGGCGAAC |
| | | GTGCGTACGTTGATCGAAAAGCTGAAATCCGGTGAGGTGG |
| | | AGTACGACTTCATAGAGGTAATGACTTGCAAAGGTGGTTGT |
| | | ATTGGTGGCGCTGGTCAACCGAAGATGAAAGCGCGTATTA |
| | | GCAATGAGATGCGTCTCAAGCGCATTGAGGGTCTGTACGA |
| | | TAAGGACAAGCACATTGCGGTCAAATGCAGCTATGAAAACC |
| | | CGGATGTCATCAACGTGTACAAGGAGTTCTTCAAACAACCG |
| | | CTGAGCCATTTATCCCACGAGTTGCTGCACACCACCTTTGA |
| | | AAGCAAGCACGATATGTTGGGTCTGAAGGATGACAACAAC |
| | | GTTTCGGATATTGGCTAGGGATCC |
| | | GGGTCGAGGAGAAGGATGAGAAAAAGTGACCAGGCATCAA |
| | | ATAAAACGAAAGGCTCAGTCGAAAGACTGGGCCTTTCGTTT |
| | | TATCTGTTGTTTGTCGGTGAACGCTCTCTACTAGAGTCACA |
| | | CTGGCTCACCTTCGGGTGGGCCTTTCTGCGTTTATATA |
| | | CTAGAGGGCTTTACACTTTATGCTTCCGGCTCGTATGTTGT |
| | | GTGGAAAGAAGGAGATATACATATGGCTCATAGTTTAAGCG |
| | | CACATTCCCGTCAGGCCGGAGATCGCAAACTGGGCGCAG |
| | | GTGCGGCATCTAGCCGCCCATCATGTCCTTCTCGCCGCAT |
| | | TGTCCGCGTGGCGGCCCATGCTTCTGCGTCCAAGGCGACT |
| | | CCCGATGTACCTGTTGACGATCTTCCTCCTGCGCATGCCC |
| | | GTGCAGCAGTAGCGGCCGCTAACCGTCGTGCGCGCGCTA |
| | | TGGCATCAGCGGAGGCCGCGGCAGAGACCCTGGGCGATT |
| | | TCCTGGGCTTAGGGAAGGGGGGGCTTTCGCCGGGCGCAA |
| | | CCGCCAACCTTGATCGTGAGCAAGTATTAGGTGTGTTGGA |
| | | GGCGGTCTGGCGTCGTGGAGACCTTAATCTGGAGCGTGC |
| | | GTTGTACAGCCATGCCAATGCGGTGACTAACAAATACTGTG |
| | | GTGGGGGGGTCTACTATCGCGGCCTTGTGGAGTTCAGTAA |
| | | TATTTGCCAAAACGATTGCTCATATTGTGGGATTCGCAACA |
| | | ATCAAAAAGAAGTTTGGCGCTATACTATGCCGGTTGAAGAG |
| | | GTGGTCGAGGTAGCTAAGTGGGCGCTTGAAAACGGCATTC |
| | | GCAACATTATGTTGCAAGGGGGTGAATTAAAGACAGAGCAA |
| | | CGCTTAGCCTACTTGGAGGCATGCGTCCGTGCGATTCGCG |
| | | AGGAGACAACGCAGTTAGACCTTGAAATGCGCGCACGTGC |
| | | CGCGAGTACGACAACAGCGGAAGCGGCAGCCAGTGCACA |
| | | GGCAGACGCCGAAGCTAAACGCGGCGAGCCAGAATTGGG |
| | | TGTGGTCGTATCCTTAAGCGTCGGAGAACTTCCTATGGAAC |
| | | AGTATGAGCGTTTGTTCCGCGCCGGCGCCCGTCGCTATCT |
| | | GATCCGCATTGAAACCTCGAATCCCGACCTGTACGCGGCA |
| | | CTTCACCCGGAGCCGATGTCCTGGCACGCGCGTGTAGAAT |
| | | GCCTGCGCAACCTGAAGAAAGCTGGGTATATGTTGGGCAC |
| | | AGGAGTGATGGTCGGCTTGCCGGGACAAACCTTACACGAC |
| | | CTGGCTGGGGATGTCATGTTCTTTCGCGACATTAAAGCGG |
| | | ACATGATCGGTATGGGCCCCTTCATTACGCAGCCTGGGAC |
| | | GCCTGCAACTGATAAGTGGACCGCGCTGTATCCGAACGCC |
| | | AATAAGAACAGCCACATGAAATCTATGTTCGATCTGACCAC |
| | | TGCTATGAATGCACTTGTACGTATTACGATGGGGAACGTAA |
| | | ATATCAGTGCTACGACTGCATTACAAGCGATTATCCCCACT |
| | | GGACGTGAAATTGCGCTTGAGCGCGGCGCAAATGTTGTCA |
| | | TGCCTATTTTAACGCCTACTCAGTATCGCGAGTCCTATCAG |
| | | TTATATGAGGGTAAGCCCTGCATTACGGATACTGCTGTTCA |
| | | GTGTCGTCGTTGCCTGGACATGCGTTTGCACTCCGTTGGC |
| | | AAAACGTCTGCGGCCGGAGTTTGGGGCGATCCTGCTTCGT |
| | | TCTTGCATCCCATCGTTGGCGTCCCAGTCCCGCACGACTT |
| | | GTCATCACCTGCTTTGGCCGCTGCTGCAAGTGCGGATTTTC |
| | | ACGAGGTTGGCGCAGGTCCCTGGAACCCCATCCGACTAGA |
| | | GCGTCTGGTTGAAGTGCCGGACCGTTACCCTGACCCCGAT |
| | | AACCACGGACGCAAAAAGGCAGGAGCCGGGAAAGGAGGC |
| | | AAGGCGCATGACTCTCACGACGATGGCGATCACGACGACC |
| | | ACCACCACCACCACGGAGCTGCCCCCGCGGGTGCAGCGG |
| | | CTGGAAAGGGTACCGGTGCAGCTGCAATTGGTGGCGGAG |
| | | CGGGGGCTAGCCGTCAACGCGTAGCAGGGGCTGCTGCCG |
| | | CCTCTGCTCGTCTGTGTGCGGGAGCTCGCCGCGCTGGCC |
| | | GTGTTGTCGCGTCGCCATTGCGCCCAGCTGCCGCTTGCCG |
| | | TGGTGTGGCCGTCAAGGCCGCAGCTGCTGCTGCGGGTGA |
| | | AGACGCTGGTGCTGGTACGTCTGGCGTAGGTTCAAATATT |
| | | GTTACGTCTCCCGGAATCGCTAGCACTACGGCACATGGTG |
| | | TTCCTCGTATCAATATTGGGGTGTTCGGCGTGATGAATGCA |
| | | GGAAAGTCTACACTTGTGAATGCTCTGGCGCAACAGGAAG |
| | | CATGCATTGTTGACTCAACCCCCGGCACGACCGCAGATGT |
| | | AAAAACAGTTTTGCTTGAGCTTCATGCCCTTGGACCAGCCA |
| | | AGTTGCTGGACACAGCCGGATTAGACGAAGTCGGTGGACT |
| | | TGGGGATAAAAAGCGCCGTAAAGCCCTGAATACGCTGAAG |
| | | GAGTGCGATGTTGCTGTGCTGGTTGTTGACACTGATACGG |
| | | CAGCCGCCGCAATCAAATCGGGACGCCTTGCTGAAGCCCT |
| | | GGAATGGGAATCGAAAGTAATGGAACAGGCACACAAGTAT |
| | | AATGTCAGTCCTGTACTGCTTCTGAATGTAAAATCACGCGG |
| | | GCTGCCTGAAGCGCAGGCAGCTAGCATGCTTGAGGCGGT |
| | | CGCTGGTATGCTTGACCCATCCAAGCAAATCCCTCGTATGT |
| | | CGCTGGATTTAGCGTCCACCCCCCTGCACGAGCGTAGTAC |
| | | GATTACGTCTGCATTCGTCAAGGAAGGAGCAGTGCGCAGT |
| | | TCACGCTATGGGGCTCCTCTGCCGGGGTGTTTGCCCCGTT |
| | | GGTCTCTTGGACGCAACGCCCGCTTACTGATGGTAATCCC |
| | | GATGGATGCCGAAACCCCAGGTGGACGTCTTCTTCGTCCT |
| | | CAAGCGCAAGTTATGGAGGAAGCAATCCGCCATTGGGCTA |
| | | CCGTGCTTTCCGTACGCCTGGATTTAGACGCCGCACGTGG |
| | | CAAATTGGGGCCCGAGGCCTGTGAGATGGAGCGTCAGCG |
| | | TTTCGACGGGGTAATTGCTATGATGGAACGTAATGACGGAC |
| | | CCACCCTGGTGGTGACAGATTCTCAAGCGATCGATGTTGTA |
| | | CACCCTTGGACCCTGGACCGCTCATCAGGACGTCCGTTGG |
| | | TTCCAATTACTACCTTTAGCATCGCTATGGCGTACCAACAA |
| | | AACGGCGGACGTTTGGACCCGTTTGTGGAAGGATTGGAAG |
| | | CATTGGAGACGTTACAAGATGGGGATCGCGTTCTTATTTCG |
| | | GAAGCGTGTAATCATAACCGTATCACCTCCGCTTGCAACGA |
| | | CATTGGAATGGTGCAGATCCCTAACAAGCTGGAGGCAGCA |
| | | CTTGGCGGGAAAAAGTTACAAATCGAACATGCGTTTGGTCG |
| | | CGAGTTTCCCGAGCTTGAGAGTGGGGGTATGGATGGATTG |
| | | AAGTTAGCGATCCATTGTGGAGGGTGTATGATCGACGCTC |
| | | AGAAAATGCAGCAGCGTATGAAAGACTTGCATGAGGCTGG |
| | | GGTGCCGGTCACTAATTATGGGGTGTTTTTCTCTTGGGCAG |
| | | CTTGGCCCGACGCCCTTCGTCGCGCATTGGAACCGTGGG |
| | | GAGTGGAGCCACCGGTAGGTACTCCTGCTACCCCCGCAGC |
| | | CGCGCCTGCTACGGCAGCGTCCGGGGTATAATACTA |
| | | CTAGAGGGCTTTACACTTTATGCTTCCGGCTCGTATGTTGT |
| | | GTGGA |
| | | AAGAAGGAGATATACATATGTCGGTCCCCCTACAGTGCAAT |
| | | GCAGGCCGTCTTTTGGCGGGCCAGCGCCCCTGCGGCGTC |
| | | CGCGCGCGTCTGAACCGCCGTGTATGTGTTCCAGTGACCG |
| | | CACACGGCAAAGCATCTGCCACGCGCGAATACGCTGGTGA |
| | | CTTTCTTCCAGGGACCACTATTAGTCATGCGTGGTCCGTGG |
| | | AGCGTGAAACTCATCACCGTTATCGTAATCCGGCCGAGTG |
| | | GATTAACGAGGCTGCCATCCACAAGGCGCTTGAAACGTCA |
| | | AAGGCTGATGCTCAGGACGCAGGACGCGTGCGTGAGATTT |
| | | TGGCGAAGGCTAAGGAAAAGGCTTTTGTTACTGAACATGC |
| | | GCCTGTAAATGCGGAAAGTAAATCCGAATTTGTACAAGGGC |
| | | TTACCTTGGAGGAGTGCGCCACCTTAATTAACGTTGATTCT |
| | | AATAATGTCGAGTTGATGAATGAAATCTTCGACACTGCGCT |
| | | TGCTATCAAAGAACGTATCTACGGGAACCGCGTCGTGCTG |
| | | TTCGCGCCCTTGTACATTGCTAATCATTGTATGAACACTTG |
| | | CACTTATTGTGCGTTTCGCTCAGCCAATAAGGGCATGGAAC |
| | | GCTCTATTCTGACAGACGATGACCTGCGTGAAGAAGTTGC |
| | | CGCCTTGCAACGTCAAGGTCATCGCCGCATTTTAGCCCTTA |
| | | CTGGAGAACATCCTAAATACACTTTCGACAATTTCTTGCATG |
| | | CAGTCAATGTAATCGCTTCCGTGAAGACAGAGCCTGAAGG |
| | | GTCCATTCGCCGTATCAATGTCGAAATCCCACCACTTAGTG |
| | | TCTCAGACATGCGCCGCCTTAAGAATACAGATTCAGTTGGC |
| | | ACGTTCGTTCTGTTTCAAGAGACATATCACCGCGACACATT |
| | | CAAGGTAATGCATCCGTCAGGTCCAAAGAGCGACTTTGATT |
| | | TTCGCGTCTTGACCCAGGACCGCGCCATGCGCGCAGGCTT |
| | | GGACGATGTTGGTATTGGTGCTTTATTCGGGTTATATGATT |
| | | ACCGTTATGAAGTTTGCGCCATGTTAATGCATAGCGAGCAT |
| | | TTGGAACGTGAGTACAACGCAGGACCACATACTATCTCTGT |
| | | TCCCCGTATGCGCCCCGCGGACGGAAGCGAGCTTAGTATC |
| | | GCGCCTCCTTATCCTGTTAATGATGCTGATTTTATGAAATTA |
| | | GTTGCGGTCCTTCGTATTGCTGTACCATACACAGGTATGAT |
| | | CCTTAGCACTCGTGAATCACCAGAAATGCGCTCGGCGCTT |
| | | CTGAAGTGTGGGATGAGTCAAATGAGTGCCGGCTCGCGCA |
| | | CTGATGTAGGAGCCTATCACAAAGATCACACGTTAAGTACA |
| | | GAGGCGAATTTGAGTAAGTTAGCTGGGCAGTTTACCTTGCA |
| | | GGATGAACGTCCAACTAATGAGATTGTTAAGTGGCTGATGG |
| | | AGGAGGGCTATGTCCCGTCTTGGTGCACAGCTTGTTACCG |
| | | TCAAGGTCGTACGGGCGAAGACTTCATGAACATCTGTAAG |
| | | GCTGGTGACATTCACGATTTCTGTCATCCCAATAGTCTGCT |
| | | TACGCTTCAAGAGTACCTTATGGATTATGCAGATCCAGACC |
| | | TTCGTAAGAAAGGCGAGCAAGTAATTGCCCGTGAGATGGG |
| | | ACCGGACGCCTCTGAGCCGTTATCGGCGCAAAGCCGTAAG |
| | | CGCCTTGAACGCAAGATGAAACAAGTATTGGAGGGGGAGC |
| | | ACGATGTATATCTTTAACCAGGCATCAAATAAAACGAAAGG |
| | | CTCAGTCGAAAGACTGGGCCTTTCGTTTTATCTGTTGTTTG |
| | | TCGGTGAACGCTCTCTACTAGAGTCACACTGGCTCACCTTC |
| | | GGGTGGGCCTTTCTGCGTTTATA |

TABLE 2
Strains and plasmids

| | Relevant characteristics | Genotype |
|---|---|---|
| Strain | | |
| DH5α | Wildtype | fhuA2::IS2 Δ(mmuP-mhpD) 169 ΔphoA8 glnX44 |
| | | ϕ80d[ΔlacZ58(M15)] rfbD1 gyrA96 luxS11 recA1 |
| | | endA1 rphWT thiE1 hsdR17 |
| DH5α-HPGC | Wildtype with HPGC | DH5α with pHPGC (Cam^{R}) |
| ΔpfkA-HGPC | Deletion of pfkA, with HGPC | DH5α ΔpfkA::KanR with pHPGC (Cam^{R}) |
| ΔgpmA-HGPC | Deletion of gpmA, with HGPC | DH5α ΔgpmA::KanR with pHPGC (Cam^{R}) |
| Plasmids | | |
| pHPGC | pSB1C3 derivative with hydA, | |
| | petF, fnr, hydEF and hydg | |
| ΔgpmM-HGPC | Deletion of gpmM, with HGPC | DH5α ΔgpmM::KanR, pHPGC (Cam^{R}) |
| Δedd-eda-HGPC | Deletion of edd-eda, with HGPC | DH5α Δedd-eda::KanR, pHPGC (Cam^{R}) |
| Δpps-HGPC | Deletion of pps, with HGPC | DH5α Δpps::KanR, pHPGC (Cam^{R}) |
| ΔgpmM-ΔpfkA- | Deletion of gpmM, pfk, with | DH5α ΔgpmM, ΔpfkA::KanR, pHPGC (Cam^{R}) |
| HGPC | HGPC | |
| ΔgpmA-ΔpfkA- | Deletion of gpmA, pfk, with | DH5α ΔgpmA, ΔpfkA::KanR, pHPGC (Cam^{R}) |
| HGPC | HGPC | |
| ΔgpmM-Δedd-eda | Deletion of gpmM, edd-eda, with | DH5α ΔgpmM, Δedd-eda::KanR, pHPGC |
| HGPC | HGPC | (Cam^{R}) |
| ΔgpmA-Δedd-eda- | Deletion of gpmA, edd-eda, with | DH5α ΔgpmA, Δedd-eda::KanR, pHPGC |
| HGPC | HGPC | (Cam^{R}) |
| ΔgpmM-Δedd-eda- | Deletion of gpmM, edd-eda, pfk, | DH5α ΔgpmM, ΔpfkA, Δedd-eda::KanR, |
| ΔpfkA-HGPC | with HGPC | pHPGC (Cam^{R}) |
| ΔgpmA-Δedd-eda- | Deletion of gpmA, edd-eda, pfk, | DH5α ΔgpmA, ΔpfkA, Δedd-eda::KanR, |
| ΔpfkA-HGPC | with HGPC | pHPGC (Cam^{R}) |
| Δzwf::zwfZm-HGPC | E. coli zwf replaced with zwf from | DH5α Δzwf::zwfZm, pHPGC (Cam^{R}) |
| | Zymomonas mobilis, with HPGC | |
| Δgnd::gndCg-HGPC | E. coli gnd replaced with gnd from | DH5α Δgnd::gndCg, pHPGC (CamR) |
| | Corynebacterium glutamicum, | |
| | with HPGC | |
| ΔgapA::gapCCa- | E. coli gapA replaced with gapC | DH5α ΔgapA::gapCCa, pHPGC (Cam^{R}) |
| HGPC | from Clostridium acetobutylicum, | |
| | with HPGC | |
| zwf::osmYp-HGPC | zwf promoter replaced with osmY | DH5α zwf::osmYp, pHPGC (CamR) |
| | promoter, with HPGC | |
| zwfp::gapAp-HGPC | zwf promoter replaced with gapA | DH5α zwf::gapAp, pHPGC (CamR) |
| | promoter, with HPGC | |
| gndp::osmYp- | gnd promoter replaced with osmY | DH5α gnd::osmYp, pHPGC (CamR) |
| HGPC | promoter, with HPGC | |
| gndp::gapAp-HGPC | gnd promoter replaced with gapA | DH5α gnd::gapAp, pHPGC (Cam^{R}) |
| | promoter, with HPGC | |
| pgip::gapAp | Pgi promoter replaced with gapA | DH5α pgip::gapAp, pHPGC (Cam^{R}) |
| | promoter, with HGPC | |
| pglp::gapAp | Pgl promoter replaced with gapA | DH5α pglp::gapAp, pHPGC (Cam^{R}) |
| | promoter, with HGPC | |

TABLE 3
Nucleic acids and corresponding proteins referred to in description, exemplary sequences

| | | | |
|---|---|---|---|
| | | | Exemplary |
| | | | amino acid |
| | | Exemplary nucleic acid | sequence |
| Gene | Protein encoded by gene | sequence accession | accession |

Heterologous sequences for expression in recombinant microorganism

| | | | |
|---|---|---|---|
| hydA | HydA (Fe—Fe hydrogenase A1 class) | AJ308413, CAC83731.1 | Q9FYU1 |
| | | XP_001693376.1. | |
| hydG | HydG (assembly protein) preferably from | DS496119, EDP05052.1 | Q6PSL4 |
| | Chlamydomonas reinhardtii | XP_001691319.1. | |
| hydEF | HydEF (assembly protein) | DS496119, EDP05198.1 | Q6PSL5 |
| | preferably from Chlamydomonas reinhardtii | XP_001691465.1. | |
| petF | Ferrodoxin | DS496124, EDP03827.1 | A8IV40 |
| | preferably from Chlamydomonas reinhardtii | XP_001692808.1. | |
| FNR | Ferredoxin NADP reductase | DS496140, EDP00292.1 | A8J6Y8 and |
| | preferably from Chlamydomonas reinhardtii | XP_001697352.1. | P53991 |
| gtfA | Sucrose phosphorylase (e.g., from Leuconostoc | NC_008531.1 | WP_011679246 |
| | mesenteroides) | (336262 . . . 337740) | |
| cscB | Sucrose permease (e.g., from another E. coli | X63740 or X81461 | P3000 |
| | strain) | | |
| cscA | Sucrose hydrolase (e.g., from another E. coli | X81461 | P40714 |
| | strain) | | |

Targets for reduced or deleted expression

| | | | |
|---|---|---|---|
| pfkA | phosphofructokinase | NC_000913, | P0A796 |
| | | EcoGene: EG10699 | |
| pps | pyruvate kinase | EcoGene: EG10759 | P23538 |
| gpmA | glycerate mutase | EcoGene: EG11699 | P62707 |
| gpmM | glycerate mutase | EcoGene: EG12296 | P37689 |
| gapA | glyceraldehyde-3-phosphate dehydrogenase | EcoGene: EG10367 | P0A9B2 |
| edd | 6-phosphogluconate dehydratase | EcoGene: EG10257 | P0ADF6 |
| eda | 2-keto-3-deoxy-6-phosphogluconate aldolase | EcoGene: EG10256 | P0A955 |

Endogenous targets for increased expression/activity

| | | | |
|---|---|---|---|
| pgm | phosphoglucomutase | EcoGene: EG12144 | P36938 |
| zwf | glucose-6-phosphate dehydrogenase | M55005, NP_416366.1, | POAC53 |
| | | NC_000913. | |
| pgl | 6-phosphogluconolactonase | U27192, NP_415288.1 | P52697 |
| | | NC_000913.3. | |
| gnd | 6-glucophosphonate dehydrogenase | K02072, NP_416533.1 | P00350 |
| | | NC_000913.3 | |
| yfjB | NAD kinase | EcoGene: EG12192 | P0A7B3 |
| sthA | soluble pyridine nucleotide transhydrogenase | EcoGene: EG11428 | P27306 |
| | (UdhA) | | |
| tktA or | Transketolase | EcoGene: EG11427 | P27302 P33570 |
| tktB | | EcoGene: EG12100 | |
| talA or | transaldolase | EcoGene: EG11797 | P0A867 P0A870 |
| talB | | EcoGene: EG11556 | |
| pgm | phosphoglucomutase | EcoGene: EG12144 | P36938 |
| xylA | Xylose isomerase | EcoGene: EG11074 | P00944 |

It will be understood that the invention disclosed and defined in this specification extends to all alternative combinations of two or more of the individual features mentioned or evident from the text or drawings. All of these different combinations constitute various alternative aspects of the invention.

1. A recombinant microorganism for producing hydrogen gas, wherein the microorganism comprises:
exogenous nucleic acid sequences encoding one or more proteins for enabling the microorganism to produce hydrogen,
wherein the one or more proteins comprise an Fe—Fe dependent hydrogenase such as HydA,
wherein the nucleic acid sequences are operably linked to one or more promoters for enabling expression of the nucleic acid sequences in the microorganism, and
wherein the microorganism comprises a genetic modification which promotes utilisation of carbon via the pentose phosphate pathway.

2. The recombinant microorganism of claim 1, wherein the exogenous nucleic acid sequences encode the proteins ferredoxin NADP reductase (FNR) and Ferredoxin.

3. (canceled)

4. The recombinant microorganism of claim 1, wherein the genetic modification which promotes utilisation of carbon via the pentose phosphate pathway reduces or inhibits the activity or levels of one or more endogenous proteins of the microorganism selected from: phosphofructokinase, pyruvate kinase, glycerate mutase, 6-phosphogluconoate dehydratase and 2-keto-3-deoxy-6-phosphogluconate aldolase.

5. The recombinant microorganism of claim 1, wherein the genetic modification which promotes utilisation of carbon via the pentose phosphate pathway increases the level or activity of one or more proteins of the pentose phosphate pathway and NADPH regulating proteins,
wherein the genetic modification is:
a) a modification of the promoter region of the gene encoding the one or more proteins or
b) replacement of an endogenous gene encoding the one or more proteins, with a heterologous gene sequence.

6. The recombinant microorganism of claim 1, wherein the exogenous nucleic acid sequences encode a ferredoxin NADP reductase (FNR) protein and Ferredoxin protein and wherein the FNR and Ferredoxin proteins are from Chlamydomonas reinhardtii or are functionally equivalent homologs or derivatives of the FNR and Ferrodoxin proteins from Chlamydomonas reinhardtii; and/or
wherein the exogenous nucleic acid sequences encode at least one assembly protein for enabling maturation and activation of the hydrogenase and wherein the at least one assembly protein is selected from HydEF and HydG or functionally equivalent homologs or derivatives thereof.

7-8. (canceled)

9. The recombinant microorganism of claim 1, wherein the Fe—Fe dependent hydrogenase is an HydA protein or a functionally equivalent homolog or derivative thereof, from a microorganism selected from the group consisting of: Chlamydomonas reinhardtii, Volvox carteri, Giardia lamblia, Entamoeba nuttalli, Ilyobacter polytrophus, Trichomonas vaginalis, Megasphaera micronuciformis, Veillonella parvula, Veillonella atypica, and Peptoclostridium bifermentans.

10. (canceled)

11. The recombinant microorganism of claim 1, wherein the microorganism is a strain of Escherichia coli (E. coli).

12. The recombinant microorganism of claim 1, wherein the exogenous nucleic acid sequences are provided in a single polynucleotide construct.

13. The recombinant microorganism of claim 1, wherein the exogenous nucleic acid sequences are codon optimised to provide for optimised expression in the microorganism

14. The recombinant microorganism of claim 1, wherein the microorganism is a recombinant E. coli cell comprising exogenous nucleic acids encoding the proteins HydEF, HydG, HydA, ferredoxin and FNR,
wherein the HydEF, HydG, Ferredoxin and FNR are from Chlamydomonas reinhardtii, or are functionally equivalent homologs or derivatives of the HydEF, HydG, Ferredoxin and FNR from Chlamydomonas reinhardtii, and
wherein a) the cell comprises a genetic modification which reduces or inhibits the activity or levels of one or more endogenous proteins selected from the group consisting of: phosphofructokinase, pyruvate kinase, glycerate mutase, 6-phosphogluconoate dehydratase and 2-keto-3-deoxy-6-phosphogluconate aldolase; and/or b) the cell comprises a genetic modification which increases the level or activity of one or more proteins of the pentose phosphate pathway and NADPH regulating proteins.

15. (canceled)

16. The recombinant microorganism of claim 1, wherein the microorganism or comprises a genetic modification which partially or completely excises the nucleic acid sequence corresponding to one or more of the genes pfkA, pps, gpmA/gpmM, edd and eda, encoding phosphofructokinase, pyruvate kinase, glycerate mutase, 6-phosphogluconoate dehydratase and 2-keto-3-deoxy-6-phosphogluconate aldolase respectively.

17-18. (canceled)

19. The recombinant microorganism of claim 1, wherein
the genetic modification which promotes utilisation of carbon via the pentose phosphate pathway increases the level or activity of one or more proteins of the pentose phosphate pathway and NADPH regulating proteins, wherein the genetic modification is a) a modification of the promoter region of the gene encoding the one or more proteins or b) replacement of an endogenous gene encoding the one or more proteins, with a heterologous gene sequence; and
wherein the one or more proteins of the pentose phosphate pathway and NADPH regulating proteins is selected from the group consisting of: phosphoglucomutase, glucose-6-phosphate dehydrogenase, 6-phosphogluconolactonase, glyceraldehyde-3-phosphate dehydrogenase 6-phosphogluconate dehydrogenase, transketolase, transldolase, NAD kinase and soluble pyridine nucleotide transhydrogenase.

20. The recombinant microorganism of claim 1, wherein the genetic modification which promotes utilisation of carbon via the pentose phosphate pathway, increases the level or activity of one or more proteins of the pentose phosphate pathway and NADPH regulating proteins, wherein the genetic modification is a) a modification of the promoter region of the gene encoding the one or more proteins or b) replacement of an endogenous gene encoding the one or more proteins, with a heterologous gene sequence; and
wherein the promoter region of the gene encoding the one or more proteins is replaced with the gapA or osmYp promoter.

21. The recombinant microorganism of claim 1, wherein the promoter of the zwf gene, encoding glucose-6-phosphate dehydrogenase, is replaced with the gapA or osmY promoter or the anaerobically induced nar or nirB promoters; and/or
wherein the promoter of the gnd gene, encoding 6-phosphogluconate dehydrogenase is replaced with the gapA or osmY promoter; and/or
wherein the promoter of the pgi gene, encoding phosphoglucomutase, is replaced with the gapA or osmY promoter; and/or
wherein the promoter of the gene pgl encoding 6-phosphogluconolactonase is replaced with the gapA or osmY promoter.

22-24. (canceled)

25. The recombinant microorganism of claim 1, wherein the gene encoding glucose-6-phosphate dehydrogenase (zwf), is replaced with the zwf gene from Zygomonas mobilis.

26. The recombinant microorganism of claim 1, wherein, the gene encoding 6-phosphogluconate dehydrogenase (gnd) is replaced with the gnd gene from Corynebacterium glutamicum; and/or
wherein the gene encoding glyceraldehyde-3-phosphate dehydrogenase (gapA) is replaced with the gapC gene from Clostridium acetobutylicum.

27. (canceled)

28. The recombinant microorganism of claim 1, wherein the recombinant microorganism comprises a nucleic acid construct encoding one or more proteins to enable the microorganism to metabolise sucrose for energy consumption, preferably wherein the nucleic acid construct comprises the genes cscA, cscB and sp genes, encoding sucrose hydrolase, sucrose permease, and sucrose phosphorylase respectively.

29. A method for producing hydrogen gas, the method comprising: providing a recombinant microorganism of claim 1, culturing the microorganism in a suitable culture medium and under suitable conditions for enabling the microorganism to produce hydrogen gas.

30-33. (canceled)

34. A device for producing electricity from hydrogen gas, comprising the microorganism of claim 1.

35. The recombinant microorganism of claim 1, wherein the microorganism is encapsulated and/or is inactivated such that the microorganism is not capable of reproduction.


SEQUENCE LISTING (40 sequences)

SEQ ID NO: 1 - DNA, 46 bp, Chlamydomonas reinhardtii
ctagagggctttacactttatgcttccggctcgtatgttgtgtgga

SEQ ID NO: 2 - DNA, 3478 bp, Chlamydomonas reinhardtii
aagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatacta

SEQ ID NO: 3 - DNA, 46 bp, Chlamydomonas reinhardtii; identical to SEQ ID NO: 1

SEQ ID NO: 4 - DNA, 1850 bp, Chlamydomonas reinhardtii
aagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 5 - DNA, 46 bp, Chlamydomonas reinhardtii; identical to SEQ ID NO: 1

SEQ ID NO: 6 - DNA, 1642 bp, Chlamydomonas reinhardtii
aagaaggagatatacatatgtccgcattagtacttaagccctgtgcggcagtatccatccgtggctcctcgtgtcgcgcacgccaagtagccccacgcgctccgcttgcagctagcacggttcgtgtcgctcttgcaaccctggaggcaccagcgcgtcgtttaggaaacgtcgcctgtgccgcagcggcaccggccgcagaggcacccttgtctcacgtccagcaagcactggccgaactggcaaagcccaaagatgaccccacgcgtaagcacgtttgcgttcaagtcgctcccgcagtgcgtgtcgctattgctgaaaccttagggcttgcgccgggcgctacgacaccgaaacaattagcagaaggcctgcgtcgcttgggctttgacgaggttttcgatacgctgttcggggccgacttgacgatcatggaagagggctcagaacttttgcaccgtctgacggagcacttggaagcacacccgcactctgatgagccgctgcctatgtttaccagctgctgtcctggttggatcgcgatgctggagaaatcatacccagaccttatcccttatgtaagttcttgcaaatcccctcaaatgatgctggctgctatggtcaaatcgtatctggcggaaaaaaaggggattgcacctaaagatatggttatggtaagcattatgccatgcacacgcaagcagagtgaggcggatcgtgattggttttgtgtcgacgcggacccaacacttcgccaattggaccatgtgatcacgaccgtagagctggggaatattttcaaagagcgtgggatcaaccttgcggaattacccgagggggagtgggacaacccaatgggggtaggctccggtgctggagtactttttggcaccactggaggggtaatggaggcggcgctgcgtactgcatacgaattatttactggaacccccttaccccgcctttcgctttcggaggtgcgcggcatggatggcattaaagagactaacatcacgatggtaccggcccctggcagcaagtttgaagaactgttaaagcaccgcgcggcagcgcgtgccgaggctgctgcacacggaacacctggtcccttggcctgggacggcggcgcagggttcacctcggaggacgggcgtgggggtatcactcttcgtgtggcggtagctaatggcttgggaaacgccaaaaagttgatcacaaagatgcaggcgggagaggcgaagtatgactttgtagaaatcatggcgtgtccagcgggatgcgttgggggcggcgggcagccacgttccaccgataaagcaattacccagaagcgccaggctgcactttacaatctggacgagaagtcgactctgcgccgctcccatgaaaacccgtctatccgtgagttatacgacacttatttgggtgagcccttagggcacaaagcacacgaacttttacatactcactatgtagctggcggggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatata

SEQ ID NO: 7 - DNA, 46 bp, Chlamydomonas reinhardtii; identical to SEQ ID NO: 1

SEQ ID NO: 8 - DNA, 392 bp, Chlamydomonas reinhardtii
aagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaa

SEQ ID NO: 9 - DNA, 1189 bp, Chlamydomonas reinhardtii
aagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatata

SEQ ID NO: 10 - DNA, 8735 bp, Artificial Sequence
  Exemplary HPGC (hydrogen producing gene cluster) with HydA from Chlamydomonas reinhardtii
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagccctgtgcggcagtatccatccgtggctcctcgtgtcgcgcacgccaagtagccccacgcgctccgcttgcagctagcacggttcgtgtcgctcttgcaaccctggaggcaccagcgcgtcgtttaggaaacgtcgcctgtgccgcagcggcaccggccgcagaggcacccttgtctcacgtccagcaagcactggccgaactggcaaagcccaaagatgaccccacgcgtaagcacgtttgcgttcaagtcgctcccgcagtgcgtgtcgctattgctgaaaccttagggcttgcgccgggcgctacgacaccgaaacaattagcagaaggcctgcgtcgcttgggctttgacgaggttttcgatacgctgttcggggccgacttgacgatcatggaagagggctcagaacttttgcaccgtctgacggagcacttggaagcacacccgcactctgatgagccgctgcctatgtttaccagctgctgtcctggttggatcgcgatgctggagaaatcatacccagaccttatcccttatgtaagttcttgcaaatcccctcaaatgatgctggctgctatggtcaaatcgtatctggcggaaaaaaaggggattgcacctaaagatatggttatggtaagcattatgccatgcacacgcaagcagagtgaggcggatcgtgattggttttgtgtcgacgcggacccaacacttcgccaattggaccatgtgatcacgaccgtagagctggggaatattttcaaagagcgtgggatcaaccttgcggaattacccgagggggagtgggacaacccaatgggggtaggctccggtgctggagtactttttggcaccactggaggggtaatggaggcggcgctgcgtactgcatacgaattatttactggaacccccttaccccgcctttcgctttcggaggtgcgcggcatggatggcattaaagagactaacatcacgatggtaccggcccctggcagcaagtttgaagaactgttaaagcaccgcgcggcagcgcgtgccgaggctgctgcacacggaacacctggtcccttggcctgggacggcggcgcagggttcacctcggaggacgggcgtgggggtatcactcttcgtgtggcggtagctaatggcttgggaaacgccaaaaagttgatcacaaagatgcaggcgggagaggcgaagtatgactttgtagaaatcatggcgtgtccagcgggatgcgttgggggcggcgggcagccacgttccaccgataaagcaattacccagaagcgccaggctgcactttacaatctggacgagaagtcgactctgcgccgctcccatgaaaacccgtctatccgtgagttatacgacacttatttgggtgagcccttagggcacaaagcacacgaacttttacatactcactatgtagctggcggggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 11 - DNA, 2070 bp, Artificial Sequence
  pSB1C3 plasmid
tactagtagcggccgctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacctgacgtctaagaaaccattattatcatgacattaacctataaaaataggcgtatcacgaggcagaatttcagataaaaaaaatccttagctttcgctaaggatgatttctggaattcgcggccgcttctagag

SEQ ID NO: 12 - DNA, 87 bp, E. coli
ttgttaaatatagatcacaattttgaaaccgctcgggatatcagcgaaaacataagcaaaagtgaatgttttaagaacattccgtaa

SEQ ID NO: 13 - DNA, 215 bp, E. coli
atattccaccagctatttgttagtgaataaaaggttgcctgtaaaattacaaaaaccttacgcagcgtcaagcggaatcgtgtcaatcattgcgacaaatcaatcctgtgcctaagcattacgcgactgactcgcctcactcttcctttgggcttgagaccacatggggtcggcgccccgaattttaaagggcaattacgataaaaatgtgattt

SEQ ID NO: 14 - DNA, 1452 bp, Corynebacterium glutamicum
agcctctacctcgctgcggtcgccagaccactctgtatggaacgaaccgtctttgtcgatgcgtttgtatgtgtgggcaccaaaaaaatcgcgctgcccttggattaacgctgctggaaggcgctcggcgcgtaatgagtcgtaataactaagactactagcaaagacgggaatcggcaaccccagttgggtggcggtcacaatgacgcggcgccagctatcgattaagtccccaagctcggatttaaagtaaggatctaacagcaacgactctaactctgcgttcgcgtcatacgcttcaacgatgcgatttaaaaatttagcgcggatgatacaaccgccgcgccaaatagtcgctaaatcacgggggtctacatcccagttgttctcatcagaaccggctttaatttcgtcgaaaccttgtgcgtaggctaccaacttagatgcgtaaagcgcacgacggacatcctcgacaaattgagccttatctacgccaagcgcttcaaggtctgtcagtaccccagcgggaagattgccctgggcggcagcacgttgtgaagttgcaccggacagggcacgggcaaatacggcctcgccaattccggtggttgcaattcccaaatccagagccgcttttactgtccagcgtcctgtacctttttgccccgccgcatccacgataacatcaatcaacggcttgcctgtctctgcgtcgacctgagataaaacctcagccgtaatttcgatcaagtaggagtcaagatcaccagcattccactccttaaatacctcggcaatttcggccggctgcattccagccgcatagcgcagcaggtgatacgcttccccaattacttgcatatcggcgtactcaatgccgttatgtaccatcttgacgaagtgtcctgctccgtctggcccgatatgcgtaacacaaggggtaccatccacgttggcagcaattgactcaagcaaagggcccaagctttcataactcttcgcaggacctcccggcataatagaaggtccgttcaaggccccttcttctccgccggaaatgcccgcacccacaaagtgcagtccacgggcactgatctctttttcgcggcggatggtatccgtatacagagcgttgccaccatcgataatgatgtcgccttcgtccattgcgtctgctaattgattgatgacggcatctgtagcgttgccggcctggaccatgatgatggcgcgacggggcttctctaaactcgctacaaactcctcgaccgttgcggagggaataaaatttccctcgctgccatgatccgcgatcaacttatccgttttgtcggtactgcggttatagactgcaacggtgttgccgttacgggcaaaattgcgggcaaggtttgagcccataaccgcaagcccaacaacgccaatttgagccagattatcaccgtttgtcat

SEQ ID NO: 15 - DNA, 1005 bp, Clostridium acetobutylicum
atggcgaagattgcaatcaatggctttgggcgtattggccgccttgcgcttcgtcgcattctggaagtaccgggtctggaagttgtggcgattaatgatctgaccgatgcaaagatgctggcacatttgtttaaatacgattcttcgcaaggacgcttcaacggggagattgaggtgaaagaaggcgcgtttgtcgtcaacggcaaagaagtcaaggtattcgccgaggccgatccagaaaagttgccgtggggagacctgggcattgatgtggtccttgagtgtacgggctttttcactaagaaagagaaggccgaggcacacgtccgtgcaggggcgaagaaggtcgtgatctctgctccagctgggaatgatcttaaaaccattgtattcaacgtcaacaatgaagatctggacgggacggaaacggtcatctcgggtgccagctgcacgacgaattgtctggctccaatggcaaaagtcttaaatgacaagtttggcattgagaaaggattcatgacaactattcatgcgtttaccaacgaccagaatacgttagatgggccgcaccgtaaaggagacctgcgtcgtgcacgtgccgcggccgtttccatcatcccaaattcaacaggtgccgcaaaagctatctcgcaagttatcccggatctggctggtaaattagatggaaatgcacaacgtgtaccggtgcctaccggttccatcactgagctggtctcagtcctgaagaaaaaggtaacggtagaagagattaatgcagcgatgaaagaagcagccgatgagtcttttgggtacaccgaagatcccattgtatccgcggacgtcgtgggaattaactacggttctttgtttgacgcaactctgactaaaattgtagacgtgaatgggtctcaattggttaagaccgctgcatggtatgataatgagatgagttacacttctcagctggtacgcacacttgcatatttcgccaagattgctaaatag

SEQ ID NO: 16 - DNA, 1455 bp, Zymomonas mobilis
gtcataccaggtcactccgtcacgttctgccaatgcgatagcggtagacggtccccaggttcccgatacgtaagtcttaggtttcatagagttagctttccacccctcacgaattccgtcaatccagacccactgggcctcaacttcgtcacggcgtacaaacagagtggcgtctccctcaatcaggtcaagcatcagacgctcataggcgatacggcgtttgcggtccttaaaaacatcagtcaacgacaagtccagccacacctcgcgcatgtgggctccgttacggtccaatccgggctctttcaccatcatcgagatttgaatagtctcgtccggttgtaagacgatgcgtaacttattaggttgcaagatgccgccactcgaactgaagatagagtgcggtacaggtttgaactgtaccacgatttcggagcgacgggccggcaagcgtttacccgtacggatgtaaaatggaacaccctgccaacgccaattgtccacatgggctttaattgctacgaaagtttcagtatcacttggttgccctaactcgtcgatgtatcctgcaacttcctttccgccagagacccccgccccgtactgcccggttacggtgtgggtaaacaccgtatcattgttgatagggcgcaaagcgcggaatactttaaccttctcatcacgcacagcattggcctccatgtgtgcggggggctccattgcgaccaacgctacaagctgaagaatatgactctgaaccatgtcacgcagggagccactaccgtcgaagtaaccaatgcgcccttcaagtccgactgtttccgcgaccgaaatctgtacgtggtcaatccccttgctattccaaaggggctcgaacaatgcgttcccaaaacgcaaggtcaaaagattctgcaccgtttctttgcccaaatagtggtcgatgcggtaaacttgcttctctgagaataccttcagtacagcatcgttaatatggtcgctggaggcaaggtcttgtcccaggggtttttccaatgccaaacgtgaggttggccccgctaatcccgcctgctttaatccagcaatggcgccttcaaaaagtgaaggcgccgtacttaagtaaattgcgatgcctttttctacgggaccacacagatccgctaatttgccgaactgggtggggtcggtgatgtccacggtagcgtaaaaaagcttatttaaaaacttcgctttagcatcgtcattcaaacggtcagaggccacgaaacggtccagcgctttctccgcaaagtcacggaatccgtcagtatcatactcggaacgcgaggtgcatacgatacgcaagtcgtccgccagtaagccgtctgcatcaagcccataaaggctaggcagcaacatgcgttggcttaaatccccggtgctaccgaacagaatcattgttgacacagtattggtcat

SEQ ID NO: 17 - DNA, 1506 bp, Artificial Sequence
  Codon optimised nucleic acid sequence encoding HydA from Chlamydomonas reinhardtii, 5′ - 3′
cccgggatgtccgcattagttctgaagccatgtgcggcggtcagtatccgcggaagcagctgccgcgcccgtcaagtcgcgccgcgcgcgccgttagcagcgtctacagttcgtgttgcattagcaacacttgaagctcctgcgcgtcgcctgggcaacgtcgcttgtgcagcagcggctccggcggcggaggcccctttgagccacgtccaacaagccttagctgaattggccaaaccgaaagatgaccccacacgcaagcatgtttgtgtgcaagtcgccccagcggtccgcgtcgctattgccgaaacacttggacttgctcccggtgccacgaccccgaagcaattggcggagggcttacgccgtctgggtttcgacgaagtattcgacacgcttttcggggcagatttgacaattatggaagaagggtcagagttgttgcatcgtttaacggaacatttggaggctcaccctcactctgacgaaccgttgcccatgtttacctcgtgttgtccgggttggattgcgatgcttgagaaatcgtacccggatttaattccttatgtgtcctcgtgcaaatctcctcaaatgatgttagccgcgatggtgaagtcatatcttgccgaaaagaagggcatcgcacctaaagacatggtgatggtgtcaatcatgccttgcacccgtaagcagtccgaggccgatcgcgactggttttgtgtggatgctgaccctacattacgccagttagatcatgttattacaaccgtagagttgggtaacatctttaaggagcgtggtatcaatttggctgagttaccggaaggtgaatgggacaacccgatgggggtgggttcgggagccggagtattatttgggaccactggcggcgtaatggaggcagcattacgtacggcctacgaactgttcacaggtaccccgctgcctcgcctgagtttgagcgaggtgcgtggtatggacggtattaaggaaacgaacatcacaatggttcctgctcccggttcaaaattcgaggaactgttgaagcatcgcgctgccgcacgtgcagaagcagccgcacacggtactcctggtccacttgcctgggacggcggggccggatttacttcagaagacggtcgtggtggaattactttacgtgtcgctgttgcgaatgggctgggtaacgctaaaaagttaattacaaaaatgcaggccggagaggcaaaatatgatttcgtagagattatggcctgtcccgctggttgcgtaggaggtgggggccagccccgttcaactgacaaagctatcacgcagaaacgtcaagcggcattatacaatctggatgaaaaatcaacgcttcgccgttcacatgagaatccatcaatccgtgaattgtacgatacttatttaggtgagcctctggggcataaggcacacgaacttctgcatactcattacgtcgcgggtggagttgaggagaaggatgaaaagaagtagggatcc

SEQ ID NO: 18 - DNA, 1266 bp, Artificial Sequence
  Codon optimised nucleic acid sequence encoding HydA (Hyd1) from Volvox carteri, 5′ - 3′
cccgggatgtacgtttgcgtcgcacaagttacgttacatgattgcttagcatgctcagggtgcatcacatctgcggaaacggttttgcttcagcaacagtccggggatgagttcttatcccgcttggcagacccgcacactactgtcgtagttaccgtctcgccccaatcgcgcacagctttggcggcttactatggattatgcccgtctcaggctcttgcccgccttgtgggctggttgaagtggcttggggttcgcgccgtatgggatctgacaacagcccgcgatcttgtattgttggaagaggcagctgagttcatgaaccgttgggtctgcatgtatgttagcgccgggcctcttccggtcatgggcagttcatgtcctggcacaggctgggtttgttatgctgaaaagacgcacgggacgcgtgtattaccctatttgagcaccacccgttcaccccaaggagcaatgggtggcttggtaaagtccctggtggcagcagcttggggcgttactccagggtctttgtaccacgtcacaattatgccgtgttatgacaagaaattggaagcatctcgcgatgaattaaccacaacggcgacaactactaccgctgcggggacagacgcggctggagcaggtgccgccgtaggcgggcctctgcctgaagtgatggtccgtcatgcaaccgcagcgcctgatccgttgcttccgggcgtggtaccagccgacgaccagctgtactcccttccacatggctccagctcgggtggatacgcggacttcgttttccgcactgccgcgcgcgagttgtggggggtagaagttccgccaggacctttaccatggcgtactcgtcgtaacgccgacttacagcctcgtgttggtgccccaggccaatctctggtcgttgcgcgtgtgtatggattccgcaatattcagacacttttacagcagcttaagcgtggacgctgcccttatcattatgtggaagtcatggcttgtcctagcgggtgtttgaacggaggaggacaaatcaaaccggggccaggggtaacgccgcaacaattaattgaacaattggagcttttgtatgacgttgcggcacgttctccagcagacaacccggcggtagcagctttatatggctcttggttaggcggtcgtccaggggcaccccccgctcgtcaactgttacatacgacatttcgcgagcgcgaaaagactgtaactgccgcaaccgtcacgaattggtagggatcc

SEQ ID NO: 19 - DNA, 1437 bp, Artificial Sequence
  Codon optimised nucleic acid sequence encoding HydA (Hyd1) from Giardia lamblia 5′ - 3′
cccgggatgcctccaaaaccccaacacgatgtaacaggcgtggactccaataacgcgatcatgattgattacgcaaagtgcattggctgcaacatgtgcatcaaagcatgcgacgtgcaaggtattggtgtttacaagcagaacgaaaaaccgaagtacccgcctatcgtgaagctgagcaccctgtttaatagcgattgtatcggctgcggtcaatgtgcaacgatttgtccggtggacgcgattgctccaaagaacaatctggaaatttacaaaggcgaatctgcttccaaaaaagttcgtgtggcgttgatcgcgccaagcactcgcgtggcgtttggagatgtcttcgggctgccgatcggtacaaataccatttactccctgattcgcatgctgaagcaatatctgggtttcgattatgtgttcgacgtgaatttcggcgcagatgaaaccaccgtcatcgacacccaagagctgttgcacttcaaacacgagggccgtggtccggtgttcaccagctgctgcccggcgtgggttaacctctgtgaaatgaagtacccggagctgctgccccaggttagcaccgctaaatcttgtgttgcaatggtagcgacccttgtaaaaagacgttgggttcaagaacatttaatcccgaagggtattgtagacagcgttgacgatgtctacgtggctgatattatgccgtgtaccgcgaaaaaggatgaaagcatgcgtccacaactgaaccgcgatgtggacatctgcctgacggtgcgtgaagttgcggagcacctgtattttctgcacggcgcgcgcttgacgctggaggaagtcgaggcggatgcgttggttttgcgtccgggccgtagcacgcagaaaaagtgggactttgacgctccgtttaacaccgtgtctggtggctcccatatctttggtaagaccggtggcgttgccgagacgtgtctccgtttcatcagctatatgaaaaagtcgccgatagaaaacgttaaggaggaattgctgaaagagttcaagacgccgggtcagctggtgcaaaccgttaagctggtcagctgcgagatcgccggtgagacttatcgtgccctgattgcgcatggcggttcagcaatcaatgccgccgcgcgtatggtgctcaataaagaggtggagtgcgacgttgttgagcagatggcctgtccgggtggctgccagaacggtggaggcatgccgaagatcaagggtaaaaaagaggcagttttgacgcgtgcgtctaccttggacatcctggacggtaaagagcgctttgcgagcgcgggcgagaacaaaactctgtggggtttcaacggctgcttaaccgaacacgaagcgcacgagttgcttcacacccattatcagcatcgcccggtggaatcgctgctgccgcagtagggatcc

SEQ ID NO: 20 - DNA, 1419 bp, Artificial Sequence
  Codon optimised nucleic acid sequence encoding HydA (Hyd1) from Entamoeba nuttalli, 5′ - 3′
cccgggatgcctccaaaaccctcacacactgtaacaggccacgaccacaaccacagcatccagtttgactggagcaagtgtatgggctgcggtatgtgtgcaaccaaatgcaccttcggcgtattggttaagcagccgccgaagatcccgccatttgtccagccgaaccgtgaaaaactgagccaagagaacaccgataaaacgcgtgtcctgatcgatgaaagcgagtgcaccggctgcggtcaatgctctctggtgtgcaactttggatctatcaccccgatcgaccacctcgttgacaccttcaaagcaaaggaggcgggcaaaaaactcgtggccatgattgccccgagcactcgccttggtgtcgctgaggctatgggtatgccgattggctccaccgctatggcgcagctggtgcattgtctgcgtctgattggttttgactacgtttttgacgtggacgcgggtgcggataagacgaccatggacgattacgcggaagttatcgaaatgaaaaaggagggtaagggtccggcgatcaccagctgctgcccggcgtggattgagctggtggagaaagaatatccggatttgatcccgaacgtgtccactgcgcgtagcccgatcggctgcttggccggttgcatcaaacgtggttgggcaaaagatgttggcatcgcggtagaggatttgtacaccgtgggtatcatgccttgtattgcgaagaagacggaatcccaacgtcagcaaattcatcaagattacgacgcaagctgtacgtctaacgagattgcggcgtacttcaagaagcacctgccaccggaagagtgcaaattcacccaggagcgcgaggaagctctggcaaagactgaggacggccaatgtgatctgccgttccgtcgcattagcggtggctccaacatttttggtaagaccggcggggtgtgtgaaaccgtgttgcgggtgattgcgcgtaatgcgggtgttgactggaatacgtgcaccgttaacaaagaggaaacattcaaacacgccgctagcggttcgaccatgaccaatctgagtgttgacatcggcggaactataatcaccggggcagtttgccatggtggttatgcaatccgtcatgcctgcgaactgattcgcaaaggtgaactgaaagtcgacgttgttgagatgatggcctgtgttggcggctgcctgggcggtgctggccagcctaagatcccgccggcgaagaagctggaaatggataagcgcagagtgatgttagatattctggaccagcaaacggacattcgtgcagcgaatgaaaacaccgacgtgctaggctggattgataaacacttcgaccaccagggtgcgcatcaacatctgcacacctatttcaccccgcgttatcagaattagggatcc

SEQ ID NO: 21 - DNA, 1413 bp, Artificial Sequence
  Codon optimised nucleic acid sequence encoding HydA (Hyd1) from Ilyobacter,polytrophus 5′ - 3′
cccgggatgaagaacaaaacagtatcaaatgataaaatagacaataaactgaactgctccgaaaacctggaaaaccgtaccatcgacaaaagcttctacaccttttcacgtgataccagcaagtgcattaagtgctacaagtgcgtgaaagtttgcaaggacactcagggcatttccgtttttcaggttgaggaggacggcacggtgggcatcaaagaggagaatatggcggccaccttgtgtatcagctgtggtcaatgtattaaagtgtgcaccgcaggtgcgctgaaggagaaatccaacatctctctgttgaaggagcagttaaataacccgaataagcacgttgtcgcgcagctgtctccaagcttcaaacacaccattggtgatggcttcggcatcagctcgggtaccgatacttccccgaaaatcatcagcgctctgaaagaaattggtttctctaaggttttcagtaccggtttcgccagcgacgtgaatatcgttgagaccagcgcggatctgaaaaaacgcctagatgaaaacggtccgttcccggtgttcacctccacgtgcacgggttggattaactatgcggaaaagttttgtccggagttcctggggctgctctctccgtgcaaaagcccgcaacagatcctgggtagcctgagcaagtcctactacgaagagagcattgacatcagcagagaaaatatctttagcgtggcgctgatgccttgcatcgccaaaaaggatgaagctaatcgttttgacatgaaggacgaatatggtaacaaagatgtggacctggtcctgaccgtgaatgaagttgctagccttttgaacaaaaagggcattgatttaaacaattactctaagttcggtacttttgataagccgatgaaatccgacactggttcttcgcgtattaaggcggttacgggtggcctggcagaggctatcctgcgcaacaccgcacacatgatcggtgaagatccgttttctgtagacctgaagaagctgcgtggtatggatggtattaagctcacgagcgtggttctgggcgggaaaaaattaaacattgcggtcgtgaacggcatcaagaacgtgccggttattctggacatgattaaagatggcattaccgagttccacttggtcgaggttatggcctgtccgggtggctgcgtcggcggcggtggtatcccgttgtcagaagacccggacattatccaaaaacgcgcagagaaaatctactcgtacgacgcgagcagcgaaattcgttgtagctgggaaaacccagatgttaaaaccctgtatagcgagtacctgaaggagccgctgggagaggagtctcaacgtttgtttcattttcattataaaaaccgccgtaccaaacgtatcttctagggatcc

SEQ ID NO: 22 - DNA, 1506 bp, Artificial Sequence
  Codon optimised nucleic acid sequence encoding HydA (Hyd1) from Volvox carteri, 5′ - 3′
cccgggatgagtgctatgctatcttcattaacaaattctcgcttgggcgcttacgtgccgcaagtggcggtaagccgttatggttccaagccggtctacgtcccgtgcgtgccgtttcgtaagtgcacctcggcggtgccagtgccggttaagacggtgcaagaaggtaattcggtcagacttatgccagcggcggcggctccggcgggtgcggcagcggacccacattggaaacaagcatttgcagagcttgataaaccgaaagccgaacgtaaagttatgatcgcacaggttgctccggcggtgcgcgttgccatctccgagagctttggtctggcaccgggggctaccactccgggtcagctggccgagtccctgcgttgtctgggttttgatatggtgttcgacaccttgtacggcgcggacctgaccattatggaagagggcactgagttgctgtcccgtctgcaagcgcaattggaagcccacccgcatagcgaagaaccgctgccgatgtttaccagctgctgtccgggctggatcgccctgttggagaagagctatccggaattaattccgtatgtgtctagctgcaagtctccgcagatgatgttaggcgctatggtgaagacctatctggcagagaagaaaggcatctctccgagcgacatctccatggtttcgattatgccgtgtgtgaggaagcaaggtgaagcggaccgtgattggttttgcagcggcggcgccggcgttcgtgatgtggaccacgttattaccacggcggagctaggcaacatcctgaaagaacgtaatatcaacctccccgaactgccggaaggcggttgggatgagccgctgggttcgggtagcggtgcgggcgtcgttttcggtacaaccggtggtgttatggaagcggcgctgcgcaccgcgtacgagttggttacccagcaaccgctgcctcgtttgaatctgagtgaggttcgcggtatggacggcattaaagaaaccgaaatcaagatggttccgcctccgggtagcaaattcgccgagcttgttgccgcacgtgcagcggcgaaggctatggatgaagcggccgcgagcgcaggcgcgattaagtgggatggtggcagcaacttcaccgcggacgacggtgctaagggcatcaccctgcgcgtggccgtggctaatggtctgggaaacgcgaaaaagctgctgacgaaaatgcagaccggtgagtgcaaatacgacttcgtggaaattatggcatgtccgtctgggtgcgttggtggtggtggtcagccgcgcagcaccgataaacaggtggcggtcaaacgtcagcaggcactgtacgaccttgacgagcgcgcaacgatccgtcgtagccacgagaacccggctgtccaaaaagcgtacgaagatttcctgggcgagccgaacagccacaaagcgcacgatctgttgcacacccattatgttccgggtggcgttgacacggagtagggatcc

SEQ ID NO: 23 - DNA, 1419 bp, Artificial Sequence
  Codon optimised nucleic acid sequence encoding HydA (Hyd1) from Trichomonas vaginalis, 5′ - 3′
cccgggatgctagcgtcaagtgcaacagctatgaaaggcttcgccaatagcctccgtatgaaagattacagcagcaccggtattaacttcgacatgactaagtgcattaactgccaaagctgcgttcgcgcctgcacgaacatcgctggccaaaacgtcttgaagagcttgacggtgaacggcaagtccgttgtgcagaccgttaccggtaaaccgctggcggagaccaactgcatcagctgcggtcaatgtacccttggctgcccgaagttcaccatcttcgaggcggacgccatcaatccagttaaagaggtcttgaccaagaaaaacggtcgtattgccgtgtgtcagattgctccggcgattcgtatcaacatggctgaagctctgggcgttccggctggtacgatctccttgggtaaggtggtgaccgcgctgaaacgtctgggttttgactacgttttcgataccaatttcgctgccgatatgaccatcgtggaggaagcgaccgaactggttcaacgtttgtctgacaaaaacgcagtgctgcctatgtttacttcatgctgtccagcgtgggttaattacgtagagaaatccgatccgagcttgatcccgcatctgagctcttgtcgtagcccgatgagcatgctgagcagcgttatcaagaacgtctttccgaaaaagattggtacgaccgcagacaaaatctacaatgtggcgatcatgccgtgcaccgcgaagaaggacgaaattcagcgcagccaatttaccatgaaggatggtaaacaagagacaggtgcggtgctgacttctcgtgaactcgcgaaaatgattaaggaggcgaagatcaacttcaaagagctgccggatacgccgtgtgataacttttattctgaagcatcgggcggcggtgcgatcttttgtgcaaccggcggcgtgatggaagccgcggtgcgcagcgcgtataagttcctgacgaagaaagagctggcaccgattgacctgcaagacgttcgcggtgttgcgagcggcgttaaactggcggaggtggatattgctggtaccaaagtaaaagtggcggttgcgcacggcattaagaacgccatgaccctgattaagaagatcaaatcgggtgaagagcagtttaaagacgttaagttcgtggaagtgatggcatgtccgggtggctgcgtggttggtggtggcagcccgaaggctaagaccaagaaagctgtccaggcacgattgaatgcaacgtactccattgacaaaagctccaaacacagaacttctcaggacaacccgcagttgttacagctgtataaagaatcgtttgaaggtaaattcggtggtcatgtcgcgcaccacctgctgcacacccattataagaatcgtaaagtaaatccgtagggatcc

SEQ ID NO: 24 - DNA, 1470 bp, Artificial Sequence
  Codon optimised nucleic acid sequence encoding HydA (Hyd1) from Megasphaera micronuciformis, 5′ - 3′
cccgggatgaaggctgtagagggatttgaatcaaaatataaattttacgacaagcgcgtgccgattgcggacgacaacccagctgtgcactttgacgaaacgaaatgcaaaaattgtaccctttgccgtcgtgcgtgtgaaactacgcaaaccgtgctggactattacagcctggaacgcaccggcgatgttccggtttgtgttcactgcggtcaatgtgcaaatgcctgcccgtttggtgccatgatggaagttgatgatacgaacttagtcaaagctgcgatcgcggacccggataaggtggtggttttccaaacggcaccggcggtacgtgtggccattgcggaagagttcggtgcggaggcgggtacattcgcacagggtaaaatgatttcagcgttgagagccctgggtggcgactacgtgttcgacaccaactttggcgctgacatgaccatcatggaagaaacttccgagctggtgcgtcgtattaccactggtaacttcgcaatgccgcagtttaccagctgctgcccggcgtgggttgagtttgctgagaccttctacgcagaatacatcccgcatctgtccagcgcgaagagcccgattctgatgcaaaacaccacggaaaaaatctggtttgccgagaaggcgggcatcgatccgaagaagatggtgacggtttgcgttaccccgtgtaccgctaaaaaagccgagatcaagcgcaaagaattgaatgctgcggctgagtactggcatattgatggcttaaaggactccgacatttgcatcacgacccgtgaactggcacgttggctgaaagcggagaacattgacttcaataccctggatgatggtatttttgatagccacctgggcgaggcgagcggtggtggcatcatcttcggcagcaccggtggtgtcatggagtccgcactgcgcagcgcgtattacttctataccggtaaaccgatgccagcggagtatataccgtatgaaccggtgcgtggcttggacggcgttaaagaggcgaccatcgacttcagcggtatctctctgcatgtcgcagtcgtgagcggtctcggcaatgcgcgtcgttttctggataaaattatggcggacggcaccttcaaagattacacctttatcgagttcatggcgtgccagggtggctgcatcaacggtggtggccaaccgaaggttaagatgcctctggttcagaaaaccaaccaggctcgcatgaatagcttgtacaaacgtgattcggaggtttcgattaaggccgcctgggaaaaccccgaaatccaggaactctacagcgacttctacggccagccgctgtctgagcgctctgagaagtatattcacaccttctttgaggataagtccggcaacctgggtgaaggtggtgctgtgaccccgcaaacgaacccattgtctccgaagtataagccgattgaatagggatcc

SEQ ID NO: 25 - DNA, 1482 bp, Artificial Sequence
  Codon optimised nucleic acid sequence encoding HydA (Hyd1) from Veillonella parvula, 5′ - 3′
cccgggatgtcaaaatatcaatttttagatagaagggtcccgattgaggacggtaacatcgctctggttcaggatttgactaagtgcaaaaattgtagcctgtgtcgtaaagcttgcgcggtcgatatgggcgtctttgactactatgatctgaccacgaatggtgaccacccgatttgcatccactgcggtcaatgtgcgtccatctgcccattcgattctattaatgaacgcagcgagatcgatgaagttaaggcggcgattgcggacccgaacaaaatcgtcatcttccagaccgcaccggctgtgcgtgtgggtcttggcgaggagttcggcttggaggcgggtacctttgtggaaggcaagatggttgcagccctgagaaagctgggcggtgactacattctggacaccaactttggtgcggacatgaccatcatggaagaggcgtccgagttgctggaacgtgttatcaactcggatgctgttctgccgcaatttaccagctgttgtccggcgtgggttaagttcgcggagacattctacccggagttcctgcctaacctgagcaccgcgaagtccccgattgccatgcaggcaccgacccagaaaacctatttcgccgaaaaaatgggtctggacgcgaagcagattgtggcggtggccgtaactccgtgtactgccaagaaatttgagattcgccgtgacgaaatgaacagcagcgcagagtattggaataccccagaaatgcgtgataccgattactgcatcaccacgcgtgagctggcaaagtggctgcgcgcagaagaaatcaactttgacgacctcgaggacagcgcatttgatccgctgatgggtgaggctagcggtggcggtattatttttggcaacaccggcggcgttatggaagctgccatgcgcgcggcgtataaaatggcaaccggtgaagatgccccccaaacccttatcccattcgaggccatcagaggcatggatggtgcgcgcgaagctgatgttgtgatcggtgacaagaccctgcacgttgcggcggtgcatggtacgggcaatttacgtaaattcattgagcgcatgcgtgcggagaacatccactacgacttcatcgaggtgatggcatgccgtggtgggtgcatcggcggcggtggccaaccgcgtgttaaattgccgatggccgacaaagcgcgcgaagctcgtatcgcgtctctatacacccgtgacgcagaagtgactgtaaaggccgcgtgcgataatccggatattcagaaattatatgcagagtttttcgacggcaaaccgatgagccataaagcacatcacatgctgcataccacgttcgtgaatcgtagcgaagatttgggtccgaacggtgcgtgcaccccggcgacgtgcccgaccagtgttccgaacctgaaaaaggctgctgaagcggcgaaggcggcggctgaagttaactcttagggatcc

SEQ ID NO: 26 - DNA, 1443 bp, Artificial Sequence
  Codon optimised nucleic acid sequence encoding HydA (Hyd1) from Veillonella atypica, 5′ - 3′
cccgggatgtcacaatttgaatttatagataaaagggtccctattgcgctcgacaacccgagcatctatcacgatatctccaaatgcaaaaactgcaccttatgccgtcgtgcgtgcgccgacgtgatgagcgttctggactactacgatctggaagcgaccggtgacgtgccagtttgtattcattgtggacaatgtgcagcggcgtgcccgtttgactcgatgcatgcaaaaagcgaattggataaggtgaaggtggctattgccgatccggataaaatcgtcgttattcagaccgctccggctgtacgcgtgtcgattggcgagggtttcggctttgaaccgggtacgtttctcgagggcaaaatggttagcgcgctgcgtaaactgggtgccgactacgtggtcgacacgaacttcggcgcggatctgaccattatggaggaggcgtctgaattggttgatcgtctgaagaacggtggcacgatcccgcaattcaccagttgctgcccggcttgggttcgttttgcggaaatctacttcccggagttgatcccaaatctgtctagcacccgtagctgcatcgcaatggaggccgctatgattaaaacctatttcgctgagaaaaagggtattaacccggcgaatatcgtgtccgtctccgttaatccgtgtactgcgaagaaggctgagacaaagcgcgtggaagaaaatgctgccgcgcgttattacgacgacgaatcccttggcatggataccgacatcagcattaccaccagagaatttatccgttggctgaacgacgaaggcgtggacttcgagtccctggaggacagccagtttgatgatcttatcggtatggaaactggcgcgtctatcatttttggtaataccggtggcgttatggaggctgctatgagaacggcgtacaaactgattacggataaggagccgccaccgtatgcactgacccacctggaggatgtgcgcggtatgaacggtgtgaaagaggcgacggttcagctgggtgatgacgtgactctgagcgttgcggtggttcacggcggtaagaacacccgtgattttttgaatgcgctgaaggagaacggcaagcactatgacttcatcgaagtcatggcatgtccgggtgggtgcattggtggcggtggccaaccgcgtaccaaactgccgcaggcggttaagaccaaagaggcgcgtatcggcggtctgtacaaggcggacgaagaatataagtacgttgccagctatgaatcaccggaaatccaagagttgtacaagaacttcttgggagaacctctgggtcacaaagcacatgaattgctgcatacccacttcaccgatcgcagcgcacagttaggcgaccgcaaagatgttgtcccggagacctgtccgaccagcccgaaatacaagggttagggatcc

SEQ ID NO: 27 - DNA, 1413 bp, Artificial Sequence
  Codon optimised nucleic acid sequence encoding HydA (Hyd1) from Peptoclostridium bifermentans, 5′ - 3′
cccgggatgaagcacctatttacagaaaaagtagttccgatcgagctggataacccgtccattcagattgactttgataagtgcatcaagtgcggtctgtgtaaacgcgtttgtgaaaacgagattggtgttaatggttatttcgacctggagaagaccggtgacatcgccatctgcatcaactgcggccaatgtgttcaggcatgtccaaaaaaggcgatcacccaggttatcgatgtggatcgcgtgaaggaagcgatcaacgatccggaaaagattgttatcttcagcacagcgccagctgtgcgtgtcgcactgggcgaagaatttaacctggaagaaggcgcgtatgttgaggacaaaatggtggacgccctgcgtaaactgggtggagattacgtttttgacgttacctttggcgccgatatgaccatcatggaagaggcgaacgaacttgtttctcgtatcaaaaacggcaaaggcaaaaccccgcaatttaccagctgctgcccgtcctgggttaagttcgcggaaacgttctatccggagttgattccgaatctgtctaccacgaaatctccgattggcattcagggtgctgtcatcaagacctatttcgcacagaaagcaaatatcgacccggagaaaatcgtgaacgtaaccattactccgtgcaccgctaagaagtacgaaattgaccgtccggagatgaatgcgagcgcaaaatacaacaaaagcgaaaatatgagagataatgacatcatcctcaccactaaggagctggcgcagtggctccgtgacgaagagatcgacttcaacgctttggagggctcgaaatttgacaacatcttgggcttgggtagcggtgcgggcattatcttcggtaatagcggtggtgtgatggaagccgcggtgcgcacggtctacaatatcctcacccatgagaacccacataaagaactgctgcactttaatccggttcgtggtctggaagacgtgaaggaggctacccttaccattggcgataccaccctgcgcctggcagcggtgcaaggcacggcgaacgtgcgtacgttgatcgaaaagctgaaatccggtgaggtggagtacgacttcatagaggtaatgacttgcaaaggtggttgtattggtggcgctggtcaaccgaagatgaaagcgcgtattagcaatgagatgcgtctcaagcgcattgagggtctgtacgataaggacaagcacattgcggtcaaatgcagctatgaaaacccggatgtcatcaacgtgtacaaggagttcttcaaacaaccgctgagccatttatcccacgagttgctgcacaccacctttgaaagcaagcacgatatgttgggtctgaaggatgacaacaacgtttcggatattggctagggatcc

SEQ ID NO: 28 - DNA, 83 bp, E. coli
ggtaaatttgatgtacatcaaatggatccccttgctgaatcgttaaggtaggcggtaatagaaaagaaatcgaggcaaaaatg

SEQ ID NO: 29 - DNA, 52 bp, E. coli
ctcttgatcgttatcaaatcccaatactgtgttagagagtataatgccctta

SEQ ID NO: 30 - DNA, 8797 bp, Artificial Sequence
  Exemplary HPGC (hydrogen producing gene cluster) with alternative codon optimised nucleic acid sequence encoding HydA from Chlamydomonas reinhardtii, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgtccgcattagttctgaagccatgtgcggcggtcagtatccgcggaagcagctgccgcgcccgtcaagtcgcgccgcgcgcgccgttagcagcgtctacagttcgtgttgcattagcaacacttgaagctcctgcgcgtcgcctgggcaacgtcgcttgtgcagcagcggctccggcggcggaggcccctttgagccacgtccaacaagccttagctgaattggccaaaccgaaagatgaccccacacgcaagcatgtttgtgtgcaagtcgccccagcggtccgcgtcgctattgccgaaacacttggacttgctcccggtgccacgaccccgaagcaattggcggagggcttacgccgtctgggtttcgacgaagtattcgacacgcttttcggggcagatttgacaattatggaagaagggtcagagttgttgcatcgtttaacggaacatttggaggctcaccctcactctgacgaaccgttgcccatgtttacctcgtgttgtccgggttggattgcgatgcttgagaaatcgtacccggatttaattccttatgtgtcctcgtgcaaatctcctcaaatgatgttagccgcgatggtgaagtcatatcttgccgaaaagaagggcatcgcacctaaagacatggtgatggtgtcaatcatgccttgcacccgtaagcagtccgaggccgatcgcgactggttttgtgtggatgctgaccctacattacgccagttagatcatgttattacaaccgtagagttgggtaacatctttaaggagcgtggtatcaatttggctgagttaccggaaggtgaatgggacaacccgatgggggtgggttcgggagccggagtattatttgggaccactggcggcgtaatggaggcagcattacgtacggcctacgaactgttcacaggtaccccgctgcctcgcctgagtttgagcgaggtgcgtggtatggacggtattaaggaaacgaacatcacaatggttcctgctcccggttcaaaattcgaggaactgttgaagcatcgcgctgccgcacgtgcagaagcagccgcacacggtactcctggtccacttgcctgggacggcggggccggatttacttcagaagacggtcgtggtggaattactttacgtgtcgctgttgcgaatgggctgggtaacgctaaaaagttaattacaaaaatgcaggccggagaggcaaaatatgatttcgtagagattatggcctgtcccgctggttgcgtaggaggtgggggccagccccgttcaactgacaaagctatcacgcagaaacgtcaagcggcattatacaatctggatgaaaaatcaacgcttcgccgttcacatgagaatccatcaatccgtgaattgtacgatacttatttaggtgagcctctggggcataaggcacacgaacttctgcatactcattacgtcgcgggtggagttgaggagaaggatgaaaagaagtagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 31 - DNA, 8557 bp, Artificial Sequence
  Exemplary HPGC (hydrogen producing gene cluster) with codon optimised nucleic acid sequence encoding HydA (Hyd1) from Volvox carteri, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgtacgtttgcgtcgcacaagttacgttacatgattgcttagcatgctcagggtgcatcacatctgcggaaacggttttgcttcagcaacagtccggggatgagttcttatcccgcttggcagacccgcacactactgtcgtagttaccgtctcgccccaatcgcgcacagctttggcggcttactatggattatgcccgtctcaggctcttgcccgccttgtgggctggttgaagtggcttggggttcgcgccgtatgggatctgacaacagcccgcgatcttgtattgttggaagaggcagctgagttcatgaaccgttgggtctgcatgtatgttagcgccgggcctcttccggtcatgggcagttcatgtcctggcacaggctgggtttgttatgctgaaaagacgcacgggacgcgtgtattaccctatttgagcaccacccgttcaccccaaggagcaatgggtggcttggtaaagtccctggtggcagcagcttggggcgttactccagggtctttgtaccacgtcacaattatgccgtgttatgacaagaaattggaagcatctcgcgatgaattaaccacaacggcgacaactactaccgctgcggggacagacgcggctggagcaggtgccgccgtaggcgggcctctgcctgaagtgatggtccgtcatgcaaccgcagcgcctgatccgttgcttccgggcgtggtaccagccgacgaccagctgtactcccttccacatggctccagctcgggtggatacgcggacttcgttttccgcactgccgcgcgcgagttgtggggggtagaagttccgccaggacctttaccatggcgtactcgtcgtaacgccgacttacagcctcgtgttggtgccccaggccaatctctggtcgttgcgcgtgtgtatggattccgcaatattcagacacttttacagcagcttaagcgtggacgctgcccttatcattatgtggaagtcatggcttgtcctagcgggtgtttgaacggaggaggacaaatcaaaccggggccaggggtaacgccgcaacaattaattgaacaattggagcttttgtatgacgttgcggcacgttctccagcagacaacccggcggtagcagctttatatggctcttggttaggcggtcgtccaggggcaccccccgctcgtcaactgttacatacgacatttcgcgagcgcgaaaagactgtaactgccgcaaccgtcacgaattggtagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 32 - DNA, 8728 bp, Artificial Sequence
  Exemplary HPGC (hydrogen producing gene cluster) with codon optimised nucleic acid sequence encoding HydA (Hyd1) from Giardia lamblia 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgcctccaaaaccccaacacgatgtaacaggcgtggactccaataacgcgatcatgattgattacgcaaagtgcattggctgcaacatgtgcatcaaagcatgcgacgtgcaaggtattggtgtttacaagcagaacgaaaaaccgaagtacccgcctatcgtgaagctgagcaccctgtttaatagcgattgtatcggctgcggtcaatgtgcaacgatttgtccggtggacgcgattgctccaaagaacaatctggaaatttacaaaggcgaatctgcttccaaaaaagttcgtgtggcgttgatcgcgccaagcactcgcgtggcgtttggagatgtcttcgggctgccgatcggtacaaataccatttactccctgattcgcatgctgaagcaatatctgggtttcgattatgtgttcgacgtgaatttcggcgcagatgaaaccaccgtcatcgacacccaagagctgttgcacttcaaacacgagggccgtggtccggtgttcaccagctgctgcccggcgtgggttaacctctgtgaaatgaagtacccggagctgctgccccaggttagcaccgctaaatcttgtgttgcaatggtagcgacccttgtaaaaagacgttgggttcaagaacatttaatcccgaagggtattgtagacagcgttgacgatgtctacgtggctgatattatgccgtgtaccgcgaaaaaggatgaaagcatgcgtccacaactgaaccgcgatgtggacatctgcctgacggtgcgtgaagttgcggagcacctgtattttctgcacggcgcgcgcttgacgctggaggaagtcgaggcggatgcgttggttttgcgtccgggccgtagcacgcagaaaaagtgggactttgacgctccgtttaacaccgtgtctggtggctcccatatctttggtaagaccggtggcgttgccgagacgtgtctccgtttcatcagctatatgaaaaagtcgccgatagaaaacgttaaggaggaattgctgaaagagttcaagacgccgggtcagctggtgcaaaccgttaagctggtcagctgcgagatcgccggtgagacttatcgtgccctgattgcgcatggcggttcagcaatcaatgccgccgcgcgtatggtgctcaataaagaggtggagtgcgacgttgttgagcagatggcctgtccgggtggctgccagaacggtggaggcatgccgaagatcaagggtaaaaaagaggcagttttgacgcgtgcgtctaccttggacatcctggacggtaaagagcgctttgcgagcgcgggcgagaacaaaactctgtggggtttcaacggctgcttaaccgaacacgaagcgcacgagttgcttcacacccattatcagcatcgcccggtggaatcgctgctgccgcagtagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 33 - DNA, 8710 bp, Artificial Sequence
  Exemplary HPGC (hydrogen producing gene cluster) with codon optimised nucleic acid sequence encoding HydA (Hyd1) from Entamoeba nuttalli, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgcctccaaaaccctcacacactgtaacaggccacgaccacaaccacagcatccagtttgactggagcaagtgtatgggctgcggtatgtgtgcaaccaaatgcaccttcggcgtattggttaagcagccgccgaagatcccgccatttgtccagccgaaccgtgaaaaactgagccaagagaacaccgataaaacgcgtgtcctgatcgatgaaagcgagtgcaccggctgcggtcaatgctctctggtgtgcaactttggatctatcaccccgatcgaccacctcgttgacaccttcaaagcaaaggaggcgggcaaaaaactcgtggccatgattgccccgagcactcgccttggtgtcgctgaggctatgggtatgccgattggctccaccgctatggcgcagctggtgcattgtctgcgtctgattggttttgactacgtttttgacgtggacgcgggtgcggataagacgaccatggacgattacgcggaagttatcgaaatgaaaaaggagggtaagggtccggcgatcaccagctgctgcccggcgtggattgagctggtggagaaagaatatccggatttgatcccgaacgtgtccactgcgcgtagcccgatcggctgcttggccggttgcatcaaacgtggttgggcaaaagatgttggcatcgcggtagaggatttgtacaccgtgggtatcatgccttgtattgcgaagaagacggaatcccaacgtcagcaaattcatcaagattacgacgcaagctgtacgtctaacgagattgcggcgtacttcaagaagcacctgccaccggaagagtgcaaattcacccaggagcgcgaggaagctctggcaaagactgaggacggccaatgtgatctgccgttccgtcgcattagcggtggctccaacatttttggtaagaccggcggggtgtgtgaaaccgtgttgcgggtgattgcgcgtaatgcgggtgttgactggaatacgtgcaccgttaacaaagaggaaacattcaaacacgccgctagcggttcgaccatgaccaatctgagtgttgacatcggcggaactataatcaccggggcagtttgccatggtggttatgcaatccgtcatgcctgcgaactgattcgcaaaggtgaactgaaagtcgacgttgttgagatgatggcctgtgttggcggctgcctgggcggtgctggccagcctaagatcccgccggcgaagaagctggaaatggataagcgcagagtgatgttagatattctggaccagcaaacggacattcgtgcagcgaatgaaaacaccgacgtgctaggctggattgataaacacttcgaccaccagggtgcgcatcaacatctgcacacctatttcaccccgcgttatcagaattagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 34 - DNA, 8704 bp, Artificial Sequence
  Exemplary HPGC (hydrogen producing gene cluster) with codon optimised nucleic acid sequence encoding HydA (Hyd1) from Ilyobacter,polytrophus 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgaagaacaaaacagtatcaaatgataaaatagacaataaactgaactgctccgaaaacctggaaaaccgtaccatcgacaaaagcttctacaccttttcacgtgataccagcaagtgcattaagtgctacaagtgcgtgaaagtttgcaaggacactcagggcatttccgtttttcaggttgaggaggacggcacggtgggcatcaaagaggagaatatggcggccaccttgtgtatcagctgtggtcaatgtattaaagtgtgcaccgcaggtgcgctgaaggagaaatccaacatctctctgttgaaggagcagttaaataacccgaataagcacgttgtcgcgcagctgtctccaagcttcaaacacaccattggtgatggcttcggcatcagctcgggtaccgatacttccccgaaaatcatcagcgctctgaaagaaattggtttctctaaggttttcagtaccggtttcgccagcgacgtgaatatcgttgagaccagcgcggatctgaaaaaacgcctagatgaaaacggtccgttcccggtgttcacctccacgtgcacgggttggattaactatgcggaaaagttttgtccggagttcctggggctgctctctccgtgcaaaagcccgcaacagatcctgggtagcctgagcaagtcctactacgaagagagcattgacatcagcagagaaaatatctttagcgtggcgctgatgccttgcatcgccaaaaaggatgaagctaatcgttttgacatgaaggacgaatatggtaacaaagatgtggacctggtcctgaccgtgaatgaagttgctagccttttgaacaaaaagggcattgatttaaacaattactctaagttcggtacttttgataagccgatgaaatccgacactggttcttcgcgtattaaggcggttacgggtggcctggcagaggctatcctgcgcaacaccgcacacatgatcggtgaagatccgttttctgtagacctgaagaagctgcgtggtatggatggtattaagctcacgagcgtggttctgggcgggaaaaaattaaacattgcggtcgtgaacggcatcaagaacgtgccggttattctggacatgattaaagatggcattaccgagttccacttggtcgaggttatggcctgtccgggtggctgcgtcggcggcggtggtatcccgttgtcagaagacccggacattatccaaaaacgcgcagagaaaatctactcgtacgacgcgagcagcgaaattcgttgtagctgggaaaacccagatgttaaaaccctgtatagcgagtacctgaaggagccgctgggagaggagtctcaacgtttgtttcattttcattataaaaaccgccgtaccaaacgtatcttctagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 35 - DNA, 8797 bp, Artificial Sequence
  Exemplary HPGC (hydrogen producing gene cluster) with codon optimised nucleic acid sequence encoding HydA (Hyd1) from Volvox carteri, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgagtgctatgctatcttcattaacaaattctcgcttgggcgcttacgtgccgcaagtggcggtaagccgttatggttccaagccggtctacgtcccgtgcgtgccgtttcgtaagtgcacctcggcggtgccagtgccggttaagacggtgcaagaaggtaattcggtcagacttatgccagcggcggcggctccggcgggtgcggcagcggacccacattggaaacaagcatttgcagagcttgataaaccgaaagccgaacgtaaagttatgatcgcacaggttgctccggcggtgcgcgttgccatctccgagagctttggtctggcaccgggggctaccactccgggtcagctggccgagtccctgcgttgtctgggttttgatatggtgttcgacaccttgtacggcgcggacctgaccattatggaagagggcactgagttgctgtcccgtctgcaagcgcaattggaagcccacccgcatagcgaagaaccgctgccgatgtttaccagctgctgtccgggctggatcgccctgttggagaagagctatccggaattaattccgtatgtgtctagctgcaagtctccgcagatgatgttaggcgctatggtgaagacctatctggcagagaagaaaggcatctctccgagcgacatctccatggtttcgattatgccgtgtgtgaggaagcaaggtgaagcggaccgtgattggttttgcagcggcggcgccggcgttcgtgatgtggaccacgttattaccacggcggagctaggcaacatcctgaaagaacgtaatatcaacctccccgaactgccggaaggcggttgggatgagccgctgggttcgggtagcggtgcgggcgtcgttttcggtacaaccggtggtgttatggaagcggcgctgcgcaccgcgtacgagttggttacccagcaaccgctgcctcgtttgaatctgagtgaggttcgcggtatggacggcattaaagaaaccgaaatcaagatggttccgcctccgggtagcaaattcgccgagcttgttgccgcacgtgcagcggcgaaggctatggatgaagcggccgcgagcgcaggcgcgattaagtgggatggtggcagcaacttcaccgcggacgacggtgctaagggcatcaccctgcgcgtggccgtggctaatggtctgggaaacgcgaaaaagctgctgacgaaaatgcagaccggtgagtgcaaatacgacttcgtggaaattatggcatgtccgtctgggtgcgttggtggtggtggtcagccgcgcagcaccgataaacaggtggcggtcaaacgtcagcaggcactgtacgaccttgacgagcgcgcaacgatccgtcgtagccacgagaacccggctgtccaaaaagcgtacgaagatttcctgggcgagccgaacagccacaaagcgcacgatctgttgcacacccattatgttccgggtggcgttgacacggagtagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 36 - DNA, 8710 bp, Artificial Sequence
  Exemplary HPGC (hydrogen producing gene cluster) with codon optimised nucleic acid sequence encoding HydA (Hyd1) from Trichomonas vaginalis, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgctagcgtcaagtgcaacagctatgaaaggcttcgccaatagcctccgtatgaaagattacagcagcaccggtattaacttcgacatgactaagtgcattaactgccaaagctgcgttcgcgcctgcacgaacatcgctggccaaaacgtcttgaagagcttgacggtgaacggcaagtccgttgtgcagaccgttaccggtaaaccgctggcggagaccaactgcatcagctgcggtcaatgtacccttggctgcccgaagttcaccatcttcgaggcggacgccatcaatccagttaaagaggtcttgaccaagaaaaacggtcgtattgccgtgtgtcagattgctccggcgattcgtatcaacatggctgaagctctgggcgttccggctggtacgatctccttgggtaaggtggtgaccgcgctgaaacgtctgggttttgactacgttttcgataccaatttcgctgccgatatgaccatcgtggaggaagcgaccgaactggttcaacgtttgtctgacaaaaacgcagtgctgcctatgtttacttcatgctgtccagcgtgggttaattacgtagagaaatccgatccgagcttgatcccgcatctgagctcttgtcgtagcccgatgagcatgctgagcagcgttatcaagaacgtctttccgaaaaagattggtacgaccgcagacaaaatctacaatgtggcgatcatgccgtgcaccgcgaagaaggacgaaattcagcgcagccaatttaccatgaaggatggtaaacaagagacaggtgcggtgctgacttctcgtgaactcgcgaaaatgattaaggaggcgaagatcaacttcaaagagctgccggatacgccgtgtgataacttttattctgaagcatcgggcggcggtgcgatcttttgtgcaaccggcggcgtgatggaagccgcggtgcgcagcgcgtataagttcctgacgaagaaagagctggcaccgattgacctgcaagacgttcgcggtgttgcgagcggcgttaaactggcggaggtggatattgctggtaccaaagtaaaagtggcggttgcgcacggcattaagaacgccatgaccctgattaagaagatcaaatcgggtgaagagcagtttaaagacgttaagttcgtggaagtgatggcatgtccgggtggctgcgtggttggtggtggcagcccgaaggctaagaccaagaaagctgtccaggcacgattgaatgcaacgtactccattgacaaaagctccaaacacagaacttctcaggacaacccgcagttgttacagctgtataaagaatcgtttgaaggtaaattcggtggtcatgtcgcgcaccacctgctgcacacccattataagaatcgtaaagtaaatccgtagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 37 - DNA, 8761 bp, Artificial Sequence
  Exemplary HPGC (hydrogen producing gene cluster) with codon optimised nucleic acid sequence encoding HydA (Hyd1) from Megasphaera micronuciformis, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgaaggctgtagagggatttgaatcaaaatataaattttacgacaagcgcgtgccgattgcggacgacaacccagctgtgcactttgacgaaacgaaatgcaaaaattgtaccctttgccgtcgtgcgtgtgaaactacgcaaaccgtgctggactattacagcctggaacgcaccggcgatgttccggtttgtgttcactgcggtcaatgtgcaaatgcctgcccgtttggtgccatgatggaagttgatgatacgaacttagtcaaagctgcgatcgcggacccggataaggtggtggttttccaaacggcaccggcggtacgtgtggccattgcggaagagttcggtgcggaggcgggtacattcgcacagggtaaaatgatttcagcgttgagagccctgggtggcgactacgtgttcgacaccaactttggcgctgacatgaccatcatggaagaaacttccgagctggtgcgtcgtattaccactggtaacttcgcaatgccgcagtttaccagctgctgcccggcgtgggttgagtttgctgagaccttctacgcagaatacatcccgcatctgtccagcgcgaagagcccgattctgatgcaaaacaccacggaaaaaatctggtttgccgagaaggcgggcatcgatccgaagaagatggtgacggtttgcgttaccccgtgtaccgctaaaaaagccgagatcaagcgcaaagaattgaatgctgcggctgagtactggcatattgatggcttaaaggactccgacatttgcatcacgacccgtgaactggcacgttggctgaaagcggagaacattgacttcaataccctggatgatggtatttttgatagccacctgggcgaggcgagcggtggtggcatcatcttcggcagcaccggtggtgtcatggagtccgcactgcgcagcgcgtattacttctataccggtaaaccgatgccagcggagtatataccgtatgaaccggtgcgtggcttggacggcgttaaagaggcgaccatcgacttcagcggtatctctctgcatgtcgcagtcgtgagcggtctcggcaatgcgcgtcgttttctggataaaattatggcggacggcaccttcaaagattacacctttatcgagttcatggcgtgccagggtggctgcatcaacggtggtggccaaccgaaggttaagatgcctctggttcagaaaaccaaccaggctcgcatgaatagcttgtacaaacgtgattcggaggtttcgattaaggccgcctgggaaaaccccgaaatccaggaactctacagcgacttctacggccagccgctgtctgagcgctctgagaagtatattcacaccttctttgaggataagtccggcaacctgggtgaaggtggtgctgtgaccccgcaaacgaacccattgtctccgaagtataagccgattgaatagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 38 - DNA, 8773 bp, Artificial Sequence
  Exemplary HPGC (hydrogen producing gene cluster) with codon optimised nucleic acid sequence encoding HydA (Hyd1) from Veillonella parvula, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgtcaaaatatcaatttttagatagaagggtcccgattgaggacggtaacatcgctctggttcaggatttgactaagtgcaaaaattgtagcctgtgtcgtaaagcttgcgcggtcgatatgggcgtctttgactactatgatctgaccacgaatggtgaccacccgatttgcatccactgcggtcaatgtgcgtccatctgcccattcgattctattaatgaacgcagcgagatcgatgaagttaaggcggcgattgcggacccgaacaaaatcgtcatcttccagaccgcaccggctgtgcgtgtgggtcttggcgaggagttcggcttggaggcgggtacctttgtggaaggcaagatggttgcagccctgagaaagctgggcggtgactacattctggacaccaactttggtgcggacatgaccatcatggaagaggcgtccgagttgctggaacgtgttatcaactcggatgctgttctgccgcaatttaccagctgttgtccggcgtgggttaagttcgcggagacattctacccggagttcctgcctaacctgagcaccgcgaagtccccgattgccatgcaggcaccgacccagaaaacctatttcgccgaaaaaatgggtctggacgcgaagcagattgtggcggtggccgtaactccgtgtactgccaagaaatttgagattcgccgtgacgaaatgaacagcagcgcagagtattggaataccccagaaatgcgtgataccgattactgcatcaccacgcgtgagctggcaaagtggctgcgcgcagaagaaatcaactttgacgacctcgaggacagcgcatttgatccgctgatgggtgaggctagcggtggcggtattatttttggcaacaccggcggcgttatggaagctgccatgcgcgcggcgtataaaatggcaaccggtgaagatgccccccaaacccttatcccattcgaggccatcagaggcatggatggtgcgcgcgaagctgatgttgtgatcggtgacaagaccctgcacgttgcggcggtgcatggtacgggcaatttacgtaaattcattgagcgcatgcgtgcggagaacatccactacgacttcatcgaggtgatggcatgccgtggtgggtgcatcggcggcggtggccaaccgcgtgttaaattgccgatggccgacaaagcgcgcgaagctcgtatcgcgtctctatacacccgtgacgcagaagtgactgtaaaggccgcgtgcgataatccggatattcagaaattatatgcagagtttttcgacggcaaaccgatgagccataaagcacatcacatgctgcataccacgttcgtgaatcgtagcgaagatttgggtccgaacggtgcgtgcaccccggcgacgtgcccgaccagtgttccgaacctgaaaaaggctgctgaagcggcgaaggcggcggctgaagttaactcttagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 39 - DNA, 8734 bp, Artificial Sequence
  Exemplary HPGC (hydrogen producing gene cluster) with codon optimised nucleic acid sequence encoding HydA (Hyd1) from Veillonella atypica, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgtcacaatttgaatttatagataaaagggtccctattgcgctcgacaacccgagcatctatcacgatatctccaaatgcaaaaactgcaccttatgccgtcgtgcgtgcgccgacgtgatgagcgttctggactactacgatctggaagcgaccggtgacgtgccagtttgtattcattgtggacaatgtgcagcggcgtgcccgtttgactcgatgcatgcaaaaagcgaattggataaggtgaaggtggctattgccgatccggataaaatcgtcgttattcagaccgctccggctgtacgcgtgtcgattggcgagggtttcggctttgaaccgggtacgtttctcgagggcaaaatggttagcgcgctgcgtaaactgggtgccgactacgtggtcgacacgaacttcggcgcggatctgaccattatggaggaggcgtctgaattggttgatcgtctgaagaacggtggcacgatcccgcaattcaccagttgctgcccggcttgggttcgttttgcggaaatctacttcccggagttgatcccaaatctgtctagcacccgtagctgcatcgcaatggaggccgctatgattaaaacctatttcgctgagaaaaagggtattaacccggcgaatatcgtgtccgtctccgttaatccgtgtactgcgaagaaggctgagacaaagcgcgtggaagaaaatgctgccgcgcgttattacgacgacgaatcccttggcatggataccgacatcagcattaccaccagagaatttatccgttggctgaacgacgaaggcgtggacttcgagtccctggaggacagccagtttgatgatcttatcggtatggaaactggcgcgtctatcatttttggtaataccggtggcgttatggaggctgctatgagaacggcgtacaaactgattacggataaggagccgccaccgtatgcactgacccacctggaggatgtgcgcggtatgaacggtgtgaaagaggcgacggttcagctgggtgatgacgtgactctgagcgttgcggtggttcacggcggtaagaacacccgtgattttttgaatgcgctgaaggagaacggcaagcactatgacttcatcgaagtcatggcatgtccgggtgggtgcattggtggcggtggccaaccgcgtaccaaactgccgcaggcggttaagaccaaagaggcgcgtatcggcggtctgtacaaggcggacgaagaatataagtacgttgccagctatgaatcaccggaaatccaagagttgtacaagaacttcttgggagaacctctgggtcacaaagcacatgaattgctgcatacccacttcaccgatcgcagcgcacagttaggcgaccgcaaagatgttgtcccggagacctgtccgaccagcccgaaatacaagggttagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 40 - DNA, 8704 bp, Artificial Sequence
  Exemplary HPGC (hydrogen producing gene cluster) with codon optimised nucleic acid sequence encoding HydA (Hyd1) from Peptoclostridium bifermentans, 5′ - 3′
ctagagggctttacactttatgcttccggctcgtatgttgtgtggaaagaaggagatatacatatggcgatgcgttccacatttgctgcgcgtgtcggtgccaaaccggcagtgcgtggggcgcgtcccgcgtcgcgtatgtcttgcatggcctataaggttacactgaaaacgccatcaggcgataaaacaatcgaatgtccggcggacacctatatcctggatgctgcggaggaagccgggctggaccttccctatagttgccgtgcgggcgcctgttcatcctgtgctggcaaagtggcagctggaacggtagatcaatctgatcagtcctttctggatgacgctcagatggggaacggattcgtcctgacatgtgtcgcgtatcctacaagcgactgcactatccaaactcaccaagaggaggctttatattaaaagaaggagatatacatatgcaaactgttcgcgctccagcagcttcaggtgttgccacacgtgtcgcaggtcgtcgtatgtgtcgtccggttgcggcgacgaaggcttccacggctgttaccacagacatgtcgaagcgcactgttccaaccaagttagaggaaggtgaaatgccgttgaacacgtacagtaataaagctccgttcaaggcaaaggttcgttccgtggaaaaaatcacaggaccaaaagccacaggtgagacgtgccacatcattattgaaaccgaggggaagatcccgttttgggagggacaatcgtacggtgtaattccgccggggaccaagatcaactctaagggaaaagaagtgcctcatggtactcgtctttattcgattgcttctagtcgttacggagatgacttcgatggtcaaacggcatcgctgtgtgttcgccgcgcggtatacgtcgatccagagactggaaaggaggaccccgcgaaaaaaggactgtgtagtaactttttgtgtgatgccacaccaggcacggaaatttccatgacagggcccacaggaaaagtattgcttctgccagcagacgcgaacgcgccattaatctgtgtcgcaacggggactggaatcgcgccttttcgctcattctggcgccgttgcttcatcgagaatgtcccaagttataagttcactggccttttctggttgtttatgggtgtcgctaactctgatgctaaattgtacgatgaggaactgcaagctatcgcaaaagcgtatccgggacaattccgcctggactatgccttgtctcgtgaacaaaataatcgcaagggcgggaaaatgtacatccaagataaggttgaagagtatgccgacgaaattttcgatttattggataatggggcgcacatgtacttctgcgggttaaaggggatgatgccaggcatccaagatatgttagaacgcgttgcaaaagaaaaggggctgaactacgaagagtgggtcgaggggttaaagcacaagaatcaatggcatgttgaagtctactaaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatgtccgcattagtacttaagcccgggatgaagcacctatttacagaaaaagtagttccgatcgagctggataacccgtccattcagattgactttgataagtgcatcaagtgcggtctgtgtaaacgcgtttgtgaaaacgagattggtgttaatggttatttcgacctggagaagaccggtgacatcgccatctgcatcaactgcggccaatgtgttcaggcatgtccaaaaaaggcgatcacccaggttatcgatgtggatcgcgtgaaggaagcgatcaacgatccggaaaagattgttatcttcagcacagcgccagctgtgcgtgtcgcactgggcgaagaatttaacctggaagaaggcgcgtatgttgaggacaaaatggtggacgccctgcgtaaactgggtggagattacgtttttgacgttacctttggcgccgatatgaccatcatggaagaggcgaacgaacttgtttctcgtatcaaaaacggcaaaggcaaaaccccgcaatttaccagctgctgcccgtcctgggttaagttcgcggaaacgttctatccggagttgattccgaatctgtctaccacgaaatctccgattggcattcagggtgctgtcatcaagacctatttcgcacagaaagcaaatatcgacccggagaaaatcgtgaacgtaaccattactccgtgcaccgctaagaagtacgaaattgaccgtccggagatgaatgcgagcgcaaaatacaacaaaagcgaaaatatgagagataatgacatcatcctcaccactaaggagctggcgcagtggctccgtgacgaagagatcgacttcaacgctttggagggctcgaaatttgacaacatcttgggcttgggtagcggtgcgggcattatcttcggtaatagcggtggtgtgatggaagccgcggtgcgcacggtctacaatatcctcacccatgagaacccacataaagaactgctgcactttaatccggttcgtggtctggaagacgtgaaggaggctacccttaccattggcgataccaccctgcgcctggcagcggtgcaaggcacggcgaacgtgcgtacgttgatcgaaaagctgaaatccggtgaggtggagtacgacttcatagaggtaatgacttgcaaaggtggttgtattggtggcgctggtcaaccgaagatgaaagcgcgtattagcaatgagatgcgtctcaagcgcattgagggtctgtacgataaggacaagcacattgcggtcaaatgcagctatgaaaacccggatgtcatcaacgtgtacaaggagttcttcaaacaaccgctgagccatttatcccacgagttgctgcacaccacctttgaaagcaagcacgatatgttgggtctgaaggatgacaacaacgtttcggatattggctagggatccgggtcgaggagaaggatgagaaaaagtgaccaggcatcaaataaaacgaaaggctcagtcgaaagactgggcctttcgttttatctgttgtttgtcggtgaacgctctctactagagtcacactggctcaccttcgggtgggcctttctgcgtttatatactagagggctttacactttatgcttccggctcgtatgttgtgtggaaagaaggagatatacatatggctcatagtttaagcgcacattcccgtcaggccggagatcgcaaactgggcgcaggtgcggcatctagccgcccatcatgtccttctcgccgcattgtccgcgtggcggcccatgcttctgcgtccaaggcgactcccgatgtacctgttgacgatcttcctcctgcgcatgcccgtgcagcagtagcggccgctaaccgtcgtgcgcgcgctatggcatcagcggaggccgcggcagagaccctgggcgatttcctgggcttagggaagggggggctttcgccgggcgcaaccgccaaccttgatcgtgagcaagtattaggtgtgttggaggcggtctggcgtcgtggagaccttaatctggagcgtgcgttgtacagccatgccaatgcggtgactaacaaatactgtggtgggggggtctactatcgcggccttgtggagttcagtaatatttgccaaaacgattgctcatattgtgggattcgcaacaatcaaaaagaagtttggcgctatactatgccggttgaagaggtggtcgaggtagctaagtgggcgcttgaaaacggcattcgcaacattatgttgcaagggggtgaattaaagacagagcaacgcttagcctacttggaggcatgcgtccgtgcgattcgcgaggagacaacgcagttagaccttgaaatgcgcgcacgtgccgcgagtacgacaacagcggaagcggcagccagtgcacaggcagacgccgaagctaaacgcggcgagccagaattgggtgtggtcgtatccttaagcgtcggagaacttcctatggaacagtatgagcgtttgttccgcgccggcgcccgtcgctatctgatccgcattgaaacctcgaatcccgacctgtacgcggcacttcacccggagccgatgtcctggcacgcgcgtgtagaatgcctgcgcaacctgaagaaagctgggtatatgttgggcacaggagtgatggtcggcttgccgggacaaaccttacacgacctggctggggatgtcatgttctttcgcgacattaaagcggacatgatcggtatgggccccttcattacgcagcctgggacgcctgcaactgataagtggaccgcgctgtatccgaacgccaataagaacagccacatgaaatctatgttcgatctgaccactgctatgaatgcacttgtacgtattacgatggggaacgtaaatatcagtgctacgactgcattacaagcgattatccccactggacgtgaaattgcgcttgagcgcggcgcaaatgttgtcatgcctattttaacgcctactcagtatcgcgagtcctatcagttatatgagggtaagccctgcattacggatactgctgttcagtgtcgtcgttgcctggacatgcgtttgcactccgttggcaaaacgtctgcggccggagtttggggcgatcctgcttcgttcttgcatcccatcgttggcgtcccagtcccgcacgacttgtcatcacctgctttggccgctgctgcaagtgcggattttcacgaggttggcgcaggtccctggaaccccatccgactagagcgtctggttgaagtgccggaccgttaccctgaccccgataaccacggacgcaaaaaggcaggagccgggaaaggaggcaaggcgcatgactctcacgacgatggcgatcacgacgaccaccaccaccaccacggagctgcccccgcgggtgcagcggctggaaagggtaccggtgcagctgcaattggtggcggagcgggggctagccgtcaacgcgtagcaggggctgctgccgcctctgctcgtctgtgtgcgggagctcgccgcgctggccgtgttgtcgcgtcgccattgcgcccagctgccgcttgccgtggtgtggccgtcaaggccgcagctgctgctgcgggtgaagacgctggtgctggtacgtctggcgtaggttcaaatattgttacgtctcccggaatcgctagcactacggcacatggtgttcctcgtatcaatattggggtgttcggcgtgatgaatgcaggaaagtctacacttgtgaatgctctggcgcaacaggaagcatgcattgttgactcaacccccggcacgaccgcagatgtaaaaacagttttgcttgagcttcatgcccttggaccagccaagttgctggacacagccggattagacgaagtcggtggacttggggataaaaagcgccgtaaagccctgaatacgctgaaggagtgcgatgttgctgtgctggttgttgacactgatacggcagccgccgcaatcaaatcgggacgccttgctgaagccctggaatgggaatcgaaagtaatggaacaggcacacaagtataatgtcagtcctgtactgcttctgaatgtaaaatcacgcgggctgcctgaagcgcaggcagctagcatgcttgaggcggtcgctggtatgcttgacccatccaagcaaatccctcgtatgtcgctggatttagcgtccacccccctgcacgagcgtagtacgattacgtctgcattcgtcaaggaaggagcagtgcgcagttcacgctatggggctcctctgccggggtgtttgccccgttggtctcttggacgcaacgcccgcttactgatggtaatcccgatggatgccgaaaccccaggtggacgtcttcttcgtcctcaagcgcaagttatggaggaagcaatccgccattgggctaccgtgctttccgtacgcctggatttagacgccgcacgtggcaaattggggcccgaggcctgtgagatggagcgtcagcgtttcgacggggtaattgctatgatggaacgtaatgacggacccaccctggtggtgacagattctcaagcgatcgatgttgtacacccttggaccctggaccgctcatcaggacgtccgttggttccaattactacctttagcatcgctatggcgtaccaacaaaacggcggacgtttggacccgtttgtggaaggattggaagcattggagacgttacaagatggggatcgcgttcttatttcggaagcgtgtaatcataaccgtatcacctccgcttgcaacgacattggaatggtgcagatccctaacaagctggaggcagcacttggcgggaaaaagttacaaatcgaacatgcgtttggtcgcgagtttcccgagcttgagagtgggggtatggatggattgaagttagcgatccattgtggagggtgtatgatcgacgctcagaaaatgcagcagcgtatgaaagacttgcatgaggctggggtgccggtcactaattatggggtgtttttctcttgggcagcttggcccgacgcccttcgtcgcgcattggaaccgtggggagtggagccaccggtaggtactcctgctacccccgcagccgcgcctgctacggcagcgtccggggtataatactactagagggctttacactttatgcttccggctcgtatgttgtgtggaaagaaggagatatacatatgtcggtccccctacagtgcaatgcaggccgtcttttggcgggccagcgcccctgcggcgtccgcgcgcgtctgaaccgccgtgtatgtgttccagtgaccgcacacggcaaagcatctgccacgcgcgaatacgctggtgactttcttccagggaccactattagtcatgcgtggtccgtggagcgtgaaactcatcaccgttatcgtaatccggccgagtggattaacgaggctgccatccacaaggcgcttgaaacgtcaaaggctgatgctcaggacgcaggacgcgtgcgtgagattttggcgaaggctaaggaaaaggcttttgttactgaacatgcgcctgtaaatgcggaaagtaaatccgaatttgtacaagggcttaccttggaggagtgcgccaccttaattaacgttgattctaataatgtcgagttgatgaatgaaatcttcgacactgcgcttgctatcaaagaacgtatctacgggaaccgcgtcgtgctgttcgcgcccttgtacattgctaatcattgtatgaacacttgcacttattgtgcgtttcgctcagccaataagggcatggaacgctctattctgacagacgatgacctgcgtgaagaagttgccgccttgcaacgtcaaggtcatcgccgcattttagcccttactggagaacatcctaaatacactttcgacaatttcttgcatgcagtcaatgtaatcgcttccgtgaagacagagcctgaagggtccattcgccgtatcaatgtcgaaatcccaccacttagtgtctcagacatgcgccgccttaagaatacagattcagttggcacgttcgttctgtttcaagagacatatcaccgcgacacattcaaggtaatgcatccgtcaggtccaaagagcgactttgattttcgcgtcttgacccaggaccgcgccatgcgcgcaggcttggacgatgttggtattggtgctttattcgggttatatgattaccgttatgaagtttgcgccatgttaatgcatagcgagcatttggaacgtgagtacaacgcaggaccacatactatctctgttccccgtatgcgccccgcggacggaagcgagcttagtatcgcgcctccttatcctgttaatgatgctgattttatgaaattagttgcggtccttcgtattgctgtaccatacacaggtatgatccttagcactcgtgaatcaccagaaatgcgctcggcgcttctgaagtgtgggatgagtcaaatgagtgccggctcgcgcactgatgtaggagcctatcacaaagatcacacgttaagtacagaggcgaatttgagtaagttagctgggcagtttaccttgcaggatgaacgtccaactaatgagattgttaagtggctgatggaggagggctatgtcccgtcttggtgcacagcttgttaccgtcaaggtcgtacgggcgaagacttcatgaacatctgtaaggctggtgacattcacgatttctgtcatcccaatagtctgcttacgcttcaagagtaccttatggattatgcagatccagaccttcgtaagaaaggcgagcaagtaattgcccgtgagatgggaccggacgcctctgagccgttatcggcgcaaagccgtaagcgccttgaacgcaagatgaaacaagtattggagggggagcacgatgtatatctttaaccaggcatcaaataaaacgaaaggctcagtcgaaagactgggcctttcgttttatctgttgtttgtcggtgaacgctctctactagagtcacactggctcaccttcgggtgggcctttctgcgtttata